(19) United States
(12) Patent Application Publication
(10) Pub. No.: US 2025/0034618 A1
(43) Pub. Date: Jan. 30, 2025

(54) METHODS AND DEVICES FOR STORING OR STABILIZING MOLECULES

(71) Applicant: Drawbridge Health, Inc., Summerville, SC (US)

(72) Inventors: Alicia Jackson, San Diego, CA (US); Dagmar Beyerlein, San Francisco, CA (US); Sonya Clark, Oakland, CA (US); Richard P. Rava, San Diego, CA (US)

(21) Appl. No.: 18/913,691

(22) Filed: Oct. 11, 2024

Related U.S. Application Data
(63) Continuation of application No. 17/520,580, filed on Nov. 5, 2021.
Application No. 17/520,580 is a continuation of application No. 16/211,866, filed on Dec. 6, 2018, now abandoned.
Application No. 16/211,866 is a continuation of application No. PCT/US2017/036448, filed on Jun. 7, 2017.
(60) Provisional application No. 62/347,019, filed on Jun. 7, 2016.
(60) Provisional application No. 62/351,902, filed on Jun. 17, 2016.
(60) Provisional application No. 62/367,059, filed on Jul. 26, 2016.
(60) Provisional application No. 62/368,841, filed on Jul. 29, 2016.
(60) Provisional application No. 62/347,023, filed on Jun. 7, 2016.
(60) Provisional application No. 62/351,867, filed on Jun. 17, 2016.
(60) Provisional application No. 62/367,061, filed on Jul. 26, 2016.
(60) Provisional application No. 62/368,859, filed on Jul. 29, 2016.
(60) Provisional application No. 62/347,026, filed on Jun. 7, 2016.
(60) Provisional application No. 62/351,869, filed on Jun. 17, 2016.
(60) Provisional application No. 62/367,064, filed on Jul. 26, 2016.
(60) Provisional application No. 62/368,885, filed on Jul. 29, 2016.
(60) Provisional application No. 62/347,030, filed on Jun. 7, 2016.
(60) Provisional application No. 62/351,856, filed on Jun. 17, 2016.
(60) Provisional application No. 62/367,069, filed on Jul. 26, 2016.
(60) Provisional application No. 62/368,898, filed on Jul. 29, 2016.

(51) Int. Cl. C12Q 1/6806; A61B 5/15; A61B 5/151; A61J 1/05; G01N 1/40; G01N 33/68

(52) U.S. Cl. CPC C12Q 1/6806; A61B 5/150755; A61J 1/05; G01N 33/6863; A61B 5/150022; A61B 5/150389; A61B 5/15105; G01N 1/405; G01N 2333/525

(57) ABSTRACT

Provided herein, are matrices and methods for the stabilization of proteins and nucleic acids. The stabilized proteins and nucleic acids described herein can be in a sample taken from a subject and can be subsequently stabilized and stored on the matrix. An analyte of interest can be concentrated and eluted for analysis from this sample. The stabilized proteins and nucleic acids described herein can be components of a sample preparation reagent, and the reagent is stored on the matrix and hydration of the matrix with a sample can result in a reaction occurring.

### CROSS REFERENCE

This application is a continuation application U.S. patent application Ser. No. 17/520,580, filed Nov. 5, 2021, which is a continuation application U.S. patent application Ser. No. 16/211,866, filed Dec. 6, 2018, which is a continuation application of International Patent Application No. PCT/US2017/036448, filed Jun. 7, 2017, which claims benefit of priority to U.S. Provisional Application No. 62/347,019, filed Jun. 7, 2016, U.S. Provisional Application No. 62/351,902, filed Jun. 17, 2016, U.S. Provisional Application No. 62/367,059, filed Jul. 26, 2016, and U.S. Provisional Application No. 62/368,841 filed Jul. 29, 2016. This application further claims benefit of priority to U.S. Provisional Application No. 62/347,023, filed Jun. 7, 2016, U.S. Provisional Application No. 62/351,867, filed Jun. 17, 2016, U.S. Provisional Application No. 62/367,061, filed Jul. 26, 2016, and U.S. Provisional Application No. 62/368,859, filed Jul. 29, 2016. This application further claims benefit of priority to U.S. Provisional Application No. 62/347,026, filed Jun. 7, 2016, U.S. Provisional Application No. 62/351,869, filed Jun. 17, 2016, U.S. Provisional Application No. 62/367,064, filed Jul. 26, 2016, and U.S. Provisional Application No. 62/368,885, filed Jul. 29, 2016. This application further claims benefit of priority to U.S. Provisional Application No. 62/347,030, filed Jun. 7, 2016, U.S. Provisional Application No. 62/351,856, filed Jun. 17, 2016, U.S. Provisional Application No. 62/367,069, filed Jul. 26, 2016, and U.S. Provisional Application 62/368,898 filed Jul. 29, 2016. These applications are herein incorporated by reference in their entireties.

### BACKGROUND

Biological molecules, such as protein and nucleic acids, can be sensitive to environmental factors and prone to degradation. Use of these biological molecules as targets of diagnostic tests or as test reagents themselves can be limited by such challenges. For example, storage of such molecules can be resource intensive and logistically challenging, and can require consistently being kept under non-ambient conditions (e.g. cold chain storage). There exists a need for better systems and methods for efficiently stabilizing and storing biological molecules in a less resource intensive manner. There exists a need for improved methods and devices for stabilizing molecules, e.g., proteins or nucleic acids, from high volume samples. There is also a need for improved methods and devices for concentrating molecules, e.g., proteins or nucleic acids, from samples. Improved methods and devices are needed for preparing molecules in samples, e.g., protein or nucleic acids, for use in downstream applications. There is also a need for improved methods and devices for analysis of cell-free or circulating nucleic acids from biological samples.

### SUMMARY

Disclosed herein, in certain embodiments, are matrices configured to selectively stabilize a nucleic acid, a protein, or a combination thereof from a sample, wherein the matrix has a non-planar structure. In some embodiments, a surface area per unit volume of the matrix is greater than 0.14 mm⁻¹. In some embodiments, the matrix comprises a plurality of inner channels and cavities. In some embodiments, the matrix comprises a 3-dimensional structure having a height, width, and length each less than or equal to 13.3 mm. In some embodiments, the matrix comprises a corrugated sheet. In some embodiments, the matrix expands with addition of the sample to the matrix. In some embodiments, the matrix comprises a spiral shape or spring. In some embodiments, the matrix comprises granules. In some embodiments, the matrix comprises fragments, wherein the fragments comprise an average diameter of less than 100 μm. In some embodiments, the matrix comprises a sponge material. In some embodiments, the matrix comprises a solid foam. In some embodiments, the sample is selected from the group consisting of: blood, plasma, serum, urine, saliva, tissue, hair, skin cells, semen, cerebrospinal fluid, and bone marrow. In some embodiments, a sample vessel preloaded with the matrix as described above is disclosed. In some embodiments, the matrix partially or completely fills the vessel. In some embodiments, the sample vessel is configured for use with a liquid handling robot. In some embodiments, a device comprising the sample vessel ad described above and a desiccant is disclosed.

Disclosed herein, in certain embodiments, are methods for stabilizing a sample comprising a nucleic acid, a protein, or a combination thereof, the method comprising: (a) providing a matrix configured to selectively stabilize the nucleic acid, the protein, or the combination thereof, wherein the matrix has a non-planar structure; and (b) contacting the sample with the matrix, wherein the contacting stabilizes the nucleic acid, the protein, or the combination thereof contacted with the matrix. In some embodiments, the matrix has a surface area per unit volume greater than 0.14 mm⁻¹. In some embodiments, the sample is selected from the group consisting of: blood, plasma, serum, urine, saliva, tissue, hair, skin cells, semen, cerebrospinal fluid, and bone marrow. In some embodiments, the ratio of a volume of the sample to a surface area of the matrix is at least 0.426 μL/mm². In some embodiments, the matrix comprises a reagent that selectively stabilizes the protein, the nucleic acid, or the combination thereof. In some embodiments, the method further comprises eluting the nucleic acid, the protein, or the combination thereof from the matrix. In some embodiments, the nucleic acid comprises DNA, RNA, or a combination thereof. In some embodiments, the nucleic acid comprises the RNA, and wherein RNA eluted from the matrix comprises an RNA integrity number (RIN) of at least 4. In some embodiments, the nucleic acid comprises the RNA, and wherein the RNA is stabilized on the matrix for 5 days or more. In some embodiments, the nucleic acid comprises the RNA, and the RNA is stabilized on the matrix for about 5 days to about 30 days. In some embodiments, the nucleic acid comprises the RNA, and the RNA is stabilized on the matrix at less than 20% relative humidity. In some embodiments, the nucleic acid comprises the RNA, and the RNA is stabilized on the matrix at a temperature of about 15° C. to about 25° C.

Disclosed herein, in certain embodiments, are matrices configured to selectively stabilize a nucleic acid, a protein, or a combination thereof, wherein the nucleic acid, the protein, or the combination thereof is a sample preparation reagent. In some embodiments, the sample preparation reagent is a reagent used for a reaction selected from the group consisting of fragmentation reaction, sequencing reaction, extension reaction, amplification reaction, hybridization reaction, immunohistochemistry reaction, ligation reaction, end repair reaction, restriction enzyme digestion, bioconjugation reaction, and adenylation reaction. In some embodiments, the matrix is configured to selectively stabilize the nucleic acid, and the nucleic acid comprises DNA, RNA, or a combination thereof. In some embodiments, the matrix is configured to selectively stabilize the nucleic acid, and the sample preparation reagent comprises primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP), deoxyuridine triphosphate (dUTP), or a combination thereof. In some embodiments, the matrix is configured to selectively stabilize a protein and the sample preparation reagent comprises T4 RNA ligase 2, T4 RNA ligase 2 trunc, T4 RNA ligase 1, T4 DNA ligase, T4 polynucleotide kinase, transposase, reverse transcriptase, exonuclease, DNA polymerase I, Phi29 polymerase, T4 DNA polymerase, Klenow DNA polymerase, Klenow fragment (3′ to 5′ exonuclease⁻), Top DNA polymerase, Taq DNA polymerase, and Pfu DNA polymerase, high fidelity DNA polymerase, DNA fragmenting enzyme, antibody, enzyme-labeled antibodies, colorimetric or fluorescent molecule labeled antibodies, radioactive antibody isotypes, or a combination thereof. In some embodiments, the matrix comprises a first region configured to selectively stabilize a nucleic acid sample preparation reagent and a second region configured to selectively stabilize a protein sample preparation reagent. In some embodiments, the matrix carries about 0.375 to about 0.5 μL of a sample preparation reagent solution per 1 mm square of matrix. In some embodiments, the matrix comprises a thiocyanate salt, one or more free radical scavengers, an oxygen scavenger, melezitose, one or more lysis reagents, or a combination thereof. In some embodiments, the matrix has a non-planar structure. In some embodiments, the matrix has a surface area per unit volume greater than 0.14 mm⁻¹.

Disclosed herein, in certain embodiments, are methods for performing a reaction comprising: (a) providing a matrix configured to selectively stabilize a nucleic acid, a protein, or a combination thereof having a sample preparation reagent comprising a nucleic acid, a protein, or a combination thereof stabilized therein; (b) adding sample to the matrix; and (c) performing a reaction using the sample preparation reagent and sample. In some embodiments, the matrix has a non-planar structure. In some embodiments, the matrix has a surface area per unit volume greater than 0.14 mm⁻¹. In some embodiments, the sample preparation reagent comprises a nucleic acid, the nucleic acid is selected from the group consisting of: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP), deoxyuridine triphosphate (dUTP), and a combination thereof. In some embodiments, the sample preparation reagent comprises a protein, the protein is selected from the group consisting of: T4 RNA ligase 2, T4 RNA ligase 2 trunc, and T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzyme, antibody, enzyme-labeled antibodies, fluorescent molecule labeled antibodies, radioactive antibody isotypes, and a combination thereof. In some embodiments, the reaction catalyzes cDNA synthesis and the sample preparation reagent comprises reverse transcriptase, RNaseH, DNA polymerase, or a combination thereof. In some embodiments, the reaction catalyzes ligation and the sample preparation reagent comprises ligase. In some embodiments, the reaction catalyzes DNA end repair, and the sample preparation reagent comprises DNA polymerase, T5 DNA exonuclease, Taq Polymerase, polynucleotide kinase, or a combination thereof. In some embodiments, the reaction adenylates 3′ ends of DNA fragments and the sample preparation reagent comprises Klenow Fragment (3′→5′ exonuclease). In some embodiments, the reaction comprises hybridization and the sample preparation reagent comprises a nucleic acid probe. In some embodiments, the reaction is selected from the group consisting of: single strand extension and amplification, and wherein the sample preparation reagent comprises primers and dNTPs. In some embodiments, the sample preparation reagent is rehydrated with a sample comprising a nucleic acid, a protein, or a combination thereof. In some embodiments, the sample is selected from the group consisting of blood, plasma, serum, urine, saliva, tissue, hair, skin cells, semen, cerebrospinal fluid, and bone marrow. In some embodiments, adding sample to the matrix comprises hydrating the matrix.

Disclosed herein, in certain embodiments, are methods comprising: (a) receiving a matrix configured to selectively stabilize a nucleic acid, a protein, or a combination thereof; and (b) impregnating the matrix with a sample preparation reagent. In some embodiments, the matrix has a non-planar structure. In some embodiments, the matrix has a surface area per unit volume greater than 0.14 mm⁻¹. In some embodiments, the impregnating comprises contacting the matrix with a solution of the sample preparation reagent. In some embodiments, the impregnating comprises saturating the matrix with a solution comprising the sample preparation reagent. In some embodiments, the method further comprises drying the matrix. In some embodiments, the sample preparation reagent comprises a protein, a nucleic acid, or a combination thereof. In some embodiments, the sample preparation reagent comprises nucleic acid, and the nucleic acid comprises DNA or RNA. In some embodiments, the sample preparation reagent comprises nucleic acid, and the nucleic acid is a nucleic acid probe. In some embodiments, the impregnating comprises synthesizing the nucleic acid probe directly on the matrix. In some embodiments, the impregnating comprises deposition of the nucleic acid probe on the matrix. In some embodiments, the impregnating comprises deposition of a tagging reagent, a binding reagent, or a combination thereof in a specific location on the matrix.

Disclosed herein, in certain embodiments, are kits comprising a first matrix configured to selectively stabilize a nucleic acid, a protein, or a combination thereof and a first sample preparation reagent stabilized therein. In some embodiments, the kit further comprises a second matrix configured to selectively stabilize a nucleic acid, a protein, or a combination thereof and a second sample preparation reagent stabilized therein, wherein the first sample preparation reagent and the second sample preparation reagent are different. In some embodiments, the first sample preparation reagent comprises primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, an oligonucleotide sequences comprising an adapter sequence and a flow cell binding site, adapter sequences configured for paired end sequencing, deoxynucleoside triphosphates (dNTPs) comprising deoxyadenosine triphosphate (dATP), deoxyguanosine triphosphate (dGTP), deoxycytidine triphosphate (dCTP), deoxythymidine triphosphate (dTTP), deoxyuridine triphosphate (dUTP), or a combination thereof. In some embodiments, the first sample preparation reagent comprises T4 RNA ligase 2, T4 RNA ligase 2 trunc, T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzymes, antibody, enzyme-labeled antibodies, fluorescent molecule labeled antibodies, radioactive antibody isotypes, or a combination thereof. In some embodiments, the second sample preparation reagent comprises primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP) or deoxyuridine triphosphate (dUTP). In some embodiments, the second sample preparation reagent comprises T4 RNA ligase 2, T4 RNA ligase 2 trunc, T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzyme, antibody, enzyme-labeled antibodies, fluorescent molecule labeled antibodies, radioactive antibody isotypes, or a combination thereof.

Disclosed herein, in certain embodiments, are method for concentrating a molecule, the method comprising: (a) applying a first volume of a sample to a matrix configured to selectively stabilize a nucleic acid, a protein, or a combination thereof, wherein the first volume comprises an analyte to be concentrated; and (b) eluting the analyte from the matrix using a second volume, wherein the second volume is less than the first volume, thereby concentrating the analyte. In some embodiments, the matrix has a non-planar structure. In some embodiments, the matrix has a surface area per unit volume greater than 0.14 mm⁻¹. In some embodiments, the method further comprises (c) washing a sample component from the sample from the matrix using a washing volume, wherein the sample component does not include the analyte. In some embodiments, the washing volume is greater than the first volume. In some embodiments, the second volume is a volume of an elution buffer. In some embodiments, the second volume is about 50% less than the first volume. In some embodiments, the second volume is about 5% to about 95% less than the first volume. In some embodiments, the first volume is 20 milliliters or less. In some embodiments, the second volume is at least 10 microliters. In some embodiments, the analyte is selectively eluted from the matrix. In some embodiments, the matrix further comprises an affinity molecule that binds the analyte. In some embodiments, the affinity molecule reversibly binds the analyte. In some embodiments, eluting the analyte further comprises eluting the analyte from the affinity molecule. In some embodiments, the affinity molecule is selected from the group consisting of: an antibody, a receptor, an antigen, an enzyme, a receptor, a nucleic acid, and a peptide. In some embodiments, the antibody is selected from the group consisting of a monoclonal antibody, a polyclonal antibody, and a trap antibody. In some embodiments, the affinity molecule is a receptor, wherein the receptor is selected from the group consisting of: Fc receptor, an antibody heavy chain binding protein, a lectin, a DNA binding protein, heparin, a histone, and a carrier protein. In some embodiments, the analyte is selected from the group consisting of a protein, a nucleic acid, an amino acid, a steroid, an oligosaccharide, and a combination thereof. In some embodiments, the matrix comprises a thiocyanate salt, one or more free radical scavengers, an oxygen scavenger, melezitose, one or more lysis reagents, or a combination thereof. In some embodiments, the analyte is an RNA. In some embodiments, the RNA, after elution from the matrix, has an RNA integrity number (RIN) of at least 4. In some embodiments, the RNA is stabilized on the matrix for 5 days or more. In some embodiments, the RNA is stabilized on the matrix for about 5 days to about 30 days. In some embodiments, the RNA is stabilized on the matrix at less than 20% relative humidity. In some embodiments, the RNA is stabilized on the matrix at a temperature of about 15° C. to about 25° C. In some embodiments, the sample is selected from the group consisting of blood, plasma, serum, urine, saliva, tissue, hair, skin cells, semen, cerebrospinal fluid, and bone marrow.

Disclosed herein, in certain embodiments, are kits comprising (a) a matrix configured to selectively stabilize an analyte from a sample; and (b) an elution buffer configured to elute the analyte from the matrix. In some embodiments, the kit further comprises a set of instructions for concentrating the analyte.

Disclosed herein, in certain embodiments, are methods for analyzing a cell-free nucleic acid comprising: (a) obtaining a sample from a subject comprising the cell-free nucleic acid; (b) contacting the sample with a matrix configured to selectively stabilize the cell-free nucleic acid; and (c) analyzing the cell-free nucleic acid. In some embodiments, the analyzing comprises determining a presence or absence of a monogenic disease. In some embodiments, the monogenic disease is cystic fibrosis, beta-thalassemia, sickle cell anemia, spinal muscular atrophy, myotonic dystrophy, fragile-X syndrome, Duchenne muscular dystrophy, Hemophilia, achondroplasia, or Huntington's disease. In some embodiments, the analyzing comprises determining a presence or absence of a fetal aneuploidy. In some embodiments, the matrix has a non-planar structure. In some embodiments, the matrix has a surface area per unit volume greater than 0.14 mm⁻¹. In some embodiments, the cell-free nucleic acid selectively stabilized on the matrix is dehydrated. In some embodiments, the subject is diagnosed with a condition or suspected of having a condition. In some embodiments, the condition is pregnancy, preeclampsia, a cancer, a neurological disease, or an autoimmune disease. In some embodiments, the cell-free nucleic acid comprises cell-free fetal DNA and cell-free maternal DNA. In some embodiments, the cell-free nucleic acid comprises cell-free DNA from a tumor cell and cell-free DNA from a non-tumor cell.

Disclosed herein, in certain embodiments, are methods of screening for a presence or absence of a fetal aneuploidy using a sample from a subject that is pregnant or suspected of being pregnant, the method comprising: (a) obtaining cell-free fetal DNA and cell-free maternal DNA from a volume of less than 5 mL of a sample from a subject that is pregnant or suspected of being pregnant; and (b) detecting a presence or absence of a fetal aneuploidy using the cell-free fetal DNA and cell-free maternal DNA from the volume of less than 5 mL of the sample. In some embodiments, the fetal aneuploidy is trisomy 21, trisomy 18, trisomy 13, or a combination thereof. In some embodiments, the method further comprises contacting the cell-free fetal DNA and the cell-free maternal DNA with a matrix configured to stabilize a nucleic acid, wherein the contacting occurs before the detecting. In some embodiments, drying the cell-free fetal DNA and cell-free maternal DNA on the matrix. In some embodiments, the method further comprises rehydrating the dried cell-free fetal DNA and cell-free maternal DNA prior to the detecting. In some embodiments, the matrix has a non-planar structure. In some embodiments, the matrix has a surface area per unit volume greater than 0.14 mm⁻¹. In some embodiments, the detecting comprises sequencing. In some embodiments, the sequencing comprises next-generation sequencing. In some embodiments, the sample is selected from the group consisting of: whole blood, plasma, urine, and cerebrospinal fluid. In some embodiments, the method further comprises filtering the sample prior to the detecting.

Disclosed herein, in certain embodiments, are methods of determining a presence or absence of a condition, or a likelihood of a condition, the method comprising: (a) selectively stabilizing a protein from a sample from a subject on a matrix configured to stabilize the protein; (b) analyzing the protein; and (c) determining a presence or absence of a condition, or a likelihood of a condition, based on the analyzing. In some embodiments, the matrix has a non-planar structure. In some embodiments, the matrix has a surface area per unit volume greater than 0.14 mm⁻¹. In some embodiments, the condition is a fetal aneuploidy, and the subject is pregnant or suspected of being pregnant. In some embodiments, the matrix comprises a thiocyanate salt, one or more free radical scavengers, an oxygen scavenger, melezitose, one or more lysis reagents, or a combination thereof. In some embodiments, the protein comprises alpha-fetoprotein (AFP), pregnancy associated plasma protein A (PAPP-A), human chorionic gonadotropin (hCG), unconjugated estriol (uE3), dimeric inhibin A (DIA), or a combination thereof. In some embodiments, the matrix is configured to reduce protein conformational changes. In some embodiments, the condition is pre-eclampsia, eclampsia, or gestational diabetes.

The systems and devices disclosed herein, comprise high surface area matrices configured to rapidly absorb sample and efficiently selectively stabilize one or more components of the sample. In some embodiments, a system, a method, or a device may comprise a high surface area matrix that selectively stabilizes nucleic acids or proteins. In some instances the matrix may be configured to comprise a planar sheet with total dimensional area (length multiplied by width) greater than 176 mm². In some instances the matrix may have a surface are per unit volume of greater than 0.14 mm⁻¹ (e.g. greater than 0.25 mm⁻¹, 0.5 mm⁻¹ 0.75 mm⁻¹, 1.0 mm⁻¹, 1.5 mm⁻¹, 3 mm⁻¹, 5 mm⁻¹, or 10 mm⁻¹). The matrix may have a non-planar structure. In further configurations the non-planar structure may have a length width and height within the same size range, and occupy a large volume of space (e.g. greater than 10 cm³, 1 cm³, 100 mm³, 10 mm³). In some instances a matrix may comprises a 3-dimensional structure having a height, width, and length each >13.3 mm. In some instances a matrix may comprises a 3-dimensional structure having a height, width, and length less than or equal to 13.3 mm. The non-planar structure may be comprised of a matrix configured for selectively stabilizing nucleic acids, metabolites, or proteins. In further embodiments, a matrix may comprise a spiral roll. A spiral roll may resemble a log, a rolled cake, or a jelly-roll shape.

In some instances a matrix that selectively stabilizes nucleic acids, metabolites, or proteins may comprise one or more dried buffers or reagents, and a matrix material configured to absorb large volumes of sample. A matrix may comprise a sponge material. In some instances the sponge material may expand upon exposure to sample. In some embodiments, the matrix may be configured to absorb greater than 100 μL (e.g. greater than 100 μL, 500 μL, 700 μL, 900 μL, 1200 μL, 1500 μL, 1800 μL, 2000 μL, 2300 μL, or 2500 μL) of sample. A matrix may comprise a plurality of inner channels and cavities. In some instances a matrix may comprise a solid foam, in further instances the foam may not become solid until it has been exposed to sample and dried. In some instances a matrix may comprise corrugated sheets. A matrix may further comprise an expanded spiral or spring. In some instances a matrix may comprise granules. In some instances the granules may be smaller than a grain of sand. In other instances the granules may be smaller than the size of a poppy seed. In further instances the granules may be smaller than the size of a dust particle or a particle of flour. In other instances matrix may comprises non-spherical fragments, for example, with an average diameter <100 μm.

In some embodiments a matrix may be enclosed or installed into a sample vessel. In further instances, the matrix may be preloaded with reagents. In some instances the matrix with any of the above shapes may fill the bottom of a vessel. In further embodiments, for example granules of matrix may be deposited in the bottom of a tube for collecting and storing sample. In some embodiments, matrix may be stored in a separate compartment. In further embodiments, a sample vessel may comprise a first compartment for matrix, a second compartment for sample, and a third compartment for rehydration buffer or reagents. In some embodiments, a sample vessel may be configured for use with a liquid handling robot.

A sample vessel, or device comprising matrix may further comprise desiccant for drying the matrix after absorption of the sample.

In some embodiments, a method for stabilizing bio-samples may comprise the steps of contacting a bio-sample with any of the previously disclosed matrix structures or components. In further embodiments, a method may be designed for stabilizing a protein or nucleic acid from a sample having a defined sample volume. In some embodiments, this method may comprise the steps of contacting a liquid sample with a matrix that selectively stabilizes the metabolite, protein or the nucleic acid, wherein the ratio of the sample volume to the matrix planar dimensional area is at least 75 μL/176 mm² or 0.426 μL/mm².

In some instances, a method for stabilizing a protein or nucleic acid may be configured for using a high surface area matrix to selectively stabilize one or more components from a liquid sample having a volume of >500 μL. Further embodiments may comprise contacting the liquid sample with a matrix that selectively stabilizes the protein or the nucleic acid.

In some instances a method for stabilizing a metabolite, protein or nucleic acid in a liquid sample may comprise the steps of contacting the liquid sample with a matrix comprising a reagent that selectively stabilizes the protein, metabolite, or the nucleic acid upon contact of the liquid sample with the matrix by hydration of the reagent such that stabilization occurs in solution. In some embodiments a chemical or electrical heater in contact with the matrix; this approach may be used to enable efficient drying.

In some aspects, a solid support matrix is provided that is configured for selectively stabilizing nucleic acids and/or proteins having a sample preparation reagent stabilized therein. In some embodiments, the sample preparation reagent is a reagent used for a reaction selected from the group consisting of: fragmentation reaction, sequencing reaction, extension reaction, amplification reaction, hybridization reaction, immunohistochemistry reaction, ligation reaction, end repair reaction, restriction enzyme digestion, bioconjugation reaction and adenylation reaction.

In some embodiments, the solid support matrix is configured to selectively stabilize nucleic acids and the sample preparation reagent comprises RNA molecules. In some embodiments, the stabilized RNA molecules comprise a RNA integrity number (RIN) of at least 4. In some embodiments, the RNA molecules are stabilized on the solid support matrix for 5 days or more. In some embodiments, RNA molecules are stabilized on the solid support matrix for 1 day or more. In some embodiments, the RNA molecules are stabilized on the solid support matrix for about 1 day to about 30 days. In some embodiments, the RNA molecules are stabilized on the solid support matrix at about 20% relative humidity. In some embodiments, the RNA molecules are stabilized on the solid support matrix at a temperature of about 4° C. to about 25° C. In some embodiments, the RNA molecules are stabilized on the solid support matrix at a temperature of about 0° C. to about 15° C.

In some embodiments, the solid support matrix is configured to selectively stabilize nucleic acids and the sample preparation reagent comprises DNA. In some embodiments, the solid support matrix is configured to selectively stabilize nucleic acids and the sample preparation reagent is selected from the group consisting of: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

In some embodiments, the solid support matrix comprises a first region configured to selectively stabilize a nucleic acid sample preparation reagent and a second region configured to selectively stabilize a protein sample preparation reagent.

In some embodiments, the solid support matrix is configured to selectively stabilize protein and the sample preparation reagent comprises protein. In some embodiments, the solid support matrix is configured to selectively stabilize protein and the sample preparation reagent is selected from the group consisting of: T4 RNA ligase 2, T4 RNA ligase 2 trunc, and T4 RNA ligase 1, T4 DNA ligase, T4 polynucleotide kinase, transposase, reverse transcriptase, exonuclease, DNA polymerase I, Phi29 polymerase, T4 DNA polymerase, Klenow DNA polymerase, Klenow fragment (3′ to 5′ exonuclease−), Top DNA polymerase, Taq DNA polymerase, and Pfu DNA polymerase, high fidelity DNA polymerase, DNA fragmenting enzyme, antibody, enzyme-labeled antibodies, colorimetric or fluorescent molecule labeled antibodies, and radioactive antibody isotypes.

In some embodiments, the solid support matrix carries about 0.375 to about 0.5 μL of a sample preparation reagent solution per 1 mm square of solid support matrix.

In some embodiments, the solid support matrix comprises a thiocyanate salt. In some embodiments, the solid support matrix comprises one or more free radical scavengers, including one or more UV inhibitors.

In some embodiments, the solid support matrix comprises an oxygen scavenger. In some embodiments, solid support matrix comprises melezitose. In some embodiments, the solid support matrix comprises one or more lysis reagents.

In yet another aspect, a method for performing a sequencing reaction is provided comprising: providing a solid support matrix configured for selectively stabilizing nucleic acids or proteins having a sample preparation reagent stabilized therein; and rehydrating the sample preparation reagent to perform a reaction.

In some embodiments, the sample preparation reagent comprises nucleic acid sample preparation reagents. In some embodiments, the nucleic acid sample preparation reagents are selected from the group consisting of: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

In some embodiments, the stabilized sample preparation reagent comprises a protein. In some embodiments, the protein is selected from the group consisting of: T4 RNA ligase 2, T4 RNA ligase 2 trunc, and T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzyme, antibody, enzyme-labeled antibodies, fluorescent molecule labeled antibodies, and radioactive antibody isotypes.

In some embodiments, the reaction catalyzes cDNA synthesis and the sample preparation reagent is one or more of the following: reverse transcriptase, RNaseH, and DNA polymerase. In some embodiments, the reaction catalyzes ligation and the sample preparation reagent comprises ligase. In some embodiments, the reaction catalyzes DNA end repair, and the sample preparation reagent comprises DNA polymerase, T5 DNA exonuclease, Taq Polymerase or polynucleotide kinase. In some embodiments, the reaction adenylates 3′ ends of DNA fragments and the sample preparation reagent comprises Klenow Fragment (3′→5′ exonuclease−). In some embodiments, the reaction comprises hybridization and the sample preparation reagent comprises nucleic acid probes. In some embodiments, the reaction is selected from the group consisting of: single strand extension and amplification, and wherein the sample preparation reagent comprises primers and dNTPs. In some embodiments, the sample preparation reagent is rehydrated with a solution comprising nucleic acids. In some embodiments, the sample preparation reagent is rehydrated with a solution comprising protein.

In yet another aspect, a method is provided comprising: receiving a solid support matrix that selectively stabilizes nucleic acids or protein; and impregnating the solid support matrix with a sample preparation reagent. In some embodiments, impregnating comprises contacting the solid support matrix with a solution of the sample preparation reagent. In some embodiments, the method further comprises drying the solid support matrix. In some embodiments, impregnating comprises saturating the solid support matrix with a solution comprising a nucleic acid reagent. In some embodiments, impregnating comprises synthesizing oligonucleotide probes directly onto the solid support matrix. In some embodiments, impregnating comprises deposition of tagging and/or binding reagents in specific locations. In some embodiments, solid support matrix is impregnated in specific regions by deposition of tagging and/or binding reagents.

In yet another aspect, a kit is provided comprising a first solid support matrix that selectively stabilizes nucleic acids or proteins and a first sample preparation reagent stabilized therein. In some embodiments, the kit comprises a second solid support matrix that selectively stabilizes nucleic acids or proteins and a second sample preparation reagent stabilized therein, wherein the first sample preparation reagent and the second sample preparation reagents are different.

In some embodiments, the first sample preparation reagent is selected from the group consisting of: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, an oligonucleotide sequences comprising an adapter sequence and a flow cell binding site, adapter sequences configured for paired end sequencing, deoxynucleoside triphosphates (dNTPs) comprising deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

In some embodiments, the first sample preparation reagent is selected from the group consisting of: T4 RNA ligase 2, T4 RNA ligase 2 trunc, and T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzymes, antibody, enzyme-labeled antibodies, fluorescent molecule labeled antibodies, and radioactive antibody isotypes.

In some embodiments, the first and the second sample preparation reagents are selected from the group consisting of: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

In some embodiments, the first and the second sample preparation reagents are selected from the group consisting of: T4 RNA ligase 2, T4 RNA ligase 2 trunc, and T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease−), Top DNA polymerase, Taq DNA polymerase, and Pfu DNA polymerase, DNA fragmenting enzyme, antibody, enzyme-labeled antibodies, fluorescent molecule labeled antibodies, and radioactive antibody isotypes.

In one aspect, a method for concentrating a molecule is provided comprising: (a) applying a first volume of a sample to a solid support matrix that selectively stabilizes nucleic acids or proteins, wherein the first volume comprises an analyte to be concentrated; and (b) eluting the analyte from the solid support matrix using a second volume, wherein the second volume is less than the first volume, thereby concentrating the analyte.

In some embodiments, the method further comprises (c) washing a sample component from the solid support matrix using a washing volume, wherein the sample component does not include the analyte. In some embodiments, the washing volume is greater than the first volume.

In some embodiments, the second volume is about 50% less than the first volume. In some embodiments, the second volume is about 5% to about 95% less than the first volume. In some embodiments, the first volume is 20 milliliters or less. In some embodiments, the second volume is at least 10 microliters.

In some embodiments, the analyte is selectively eluted from the solid support matrix.

In some embodiments, the method further comprises applying an affinity molecule to the solid support matrix that binds the analyte. In some embodiments, (b) further comprises eluting the analyte from the applied affinity molecule.

In some embodiments the solid support matrix further comprises an affinity molecule.

In some embodiments, the affinity molecule is selected from the group consisting of an antibody, a receptor, an antigen, an enzyme, a receptor, a nucleic acid, and a peptide. In some embodiments, the affinity molecule is an antibody, wherein the antibody is selected from the group consisting of: a monoclonal antibody, a polyclonal antibody, and a trap antibody. In some embodiments, the affinity molecule is a receptor, wherein the receptor is selected from the group consisting of Fc receptor, an antibody heavy chain binding protein, a lectin, a DNA binding protein, heparin, a histone, and a carrier protein.

In some embodiments, the analyte is selected from the group consisting of: a protein, a nucleic acid, an amino acid, a steroid, and an oligosaccharide.

In some embodiments, the affinity molecule is configured to reversibly bind the analyte.

In some embodiments, the solid support matrix comprises melezitose. In some embodiments, the solid support matrix comprises a cell lysis reagent.

In some embodiments, the solid support matrix stabilizes ribonucleic acid (RNA) molecules with a RNA integrity number (RIN) of at least about 4. In some embodiments, the RNA molecules are stabilized on the solid support matrix for 5 days or more. In some embodiments, the RNA molecules are stabilized on the solid support matrix for about 5 days to about 30 days. In some embodiments, the RNA molecules are stabilized on the solid support matrix at less than 20% relative humidity. In some embodiments, the RNA molecules are stabilized on the solid support matrix at a temperature of about 15° C. to about 25° C.

In some embodiments, the sample is a biological sample selected from the group consisting of blood, plasma, serum, urine, saliva, tissue, hair, skin cells, semen, cerebrospinal fluid, and bone marrow.

In yet another aspect, a dry solid support matrix is configured to stabilize a nucleic acid or a protein and concentrate one or more analytes from a sample.

In some embodiments, the dry solid support comprises an affinity molecule. In some embodiments, the affinity molecule is a member of an affinity molecule-analyte pair and the affinity molecule-analyte pair comprises an affinity interaction selected from the group consisting of antibody/antigen, enzyme/substrate, receptor/ligand, nucleic acid/nucleic acid binding protein, nucleic acid/complementary base sequence, nucleic acid/histone, hormone/receptor, hormone/carrier protein, glutathione/glutathione-S-transferase, and metal ions/histidine fusion proteins.

In some embodiments, the affinity molecule is configured to reversibly bind an analyte. In some embodiments, the affinity molecule is selected from the group consisting of an antibody, antigen, an enzyme, a receptor, a nucleic acid, and a peptide. In some embodiments, the analyte is selected from the group consisting of a protein, a nucleic acid, an amino acid, a steroid, and an oligosaccharide. In some embodiments, the analyte is configured to be selectively eluted from the affinity molecule. In some embodiments, the dry solid support matrix comprises melezitose. In some embodiments, the dry solid support matrix comprises a cell lysis reagent. In some embodiments, the dry solid support matrix stabilizes ribonucleic acid (RNA) molecules with a RNA integrity number (RIN) of at least 4.

In some embodiments, the RNA molecules are stabilized on the dry solid support matrix for 5 days or more. In some embodiments, the RNA molecules are stabilized on the dry solid support matrix for about 5 days to about 30 days. In some embodiments, the RNA molecules are stabilized on the dry solid support matrix comprising a protein denaturant. In some embodiments, the RNA molecules are stabilized on the dry solid support matrix at less than 20% relative humidity. In some embodiments, the RNA molecules are stabilized on the dry solid support matrix at a temperature between about 0° C. to about 25° C.

In some embodiments, the sample is a biological sample selected from the group consisting of: blood, plasma, serum, urine, saliva, tissue, hair, skin cells, semen, cerebrospinal fluid, and bone marrow.

In yet another aspect, a kit comprises: (a) a dry solid support matrix configured to stabilize a nucleic acid or a protein and concentrate an analyte from a sample; (b) an elution buffer configured to elute the analyte from the dry solid support matrix; and (c) a set of instructions for concentrating analyte.

In some embodiments, disclosed herein are methods for analyzing cell-free nucleic acids from a cell-free sample from a subject comprising: (a) obtaining dehydrated cell-free nucleic acids selectively stabilized on a solid matrix that selectively stabilizes nucleic acids, wherein the cell-free nucleic acids are from a cell-free sample from a subject; and (b) analyzing the cell-free nucleic acids. In some embodiments, the subject may be diagnosed with a condition or suspected of having a condition. In some embodiments, the condition is pregnancy. In some embodiments, the cell-free nucleic acids comprise cell-free fetal DNA and cell-free maternal DNA. In some embodiments, the method further comprises determining a presence or absence of a fetal aneuploidy based on the analyzing. In some embodiments, the analyzing comprises analysis for a monogenic disease. In some embodiments, the monogenic disease is cystic fibrosis, beta-thalassemia, sickle cell anemia, spinal muscular atrophy, myotonic dystrophy, fragile-X syndrome, Duchenne muscular dystrophy, Hemophilia, achondroplasia, or Huntington's disease. In some embodiments, the condition is preeclampsia. In some embodiments, the condition is a cancer, neurological disease, or autoimmune disease. In some embodiments, the condition is a cancer. In some embodiments, the cell-free nucleic acids comprise cell-free DNA from a tumor cell and cell-free DNA from a non-tumor cell.

In some embodiments, disclosed herein are methods of screening for a presence or absence of a fetal aneuploidy using a liquid sample from a subject that is pregnant or suspected of being pregnant, the method comprising (a) obtaining cell-free fetal DNA and cell-free maternal DNA from a volume of less than 5 mL of a liquid sample from a subject that is pregnant or suspected of being pregnant; and (b) detecting a presence or absence of a fetal aneuploidy using the cell-free fetal DNA and cell-free maternal DNA from the volume of less than 5 mL of the liquid sample. In some embodiments, the fetal aneuploidy comprises trisomy 21. In some embodiments, the fetal aneuploidy comprises trisomy 18. In some embodiments, the fetal aneuploidy comprises trisomy 13. In some embodiments, the volume is less than 3 mL of the liquid sample. In some embodiments, the volume is less than 1 mL of the liquid sample. In some embodiments, the volume is less than 150 μL of the liquid sample. In some embodiments, the method further comprises stabilizing the cell-free fetal DNA and cell-free maternal DNA on a solid matrix before the detecting. In some embodiments, the method further comprises selectively stabilizing the cell-free fetal DNA and cell-free maternal DNA on the solid matrix. In some embodiments, the method further comprises drying the cell-free fetal DNA and cell-free maternal DNA on a solid matrix prior to the detecting. In some embodiments, the method further comprises rehydrating the dried cell-free fetal DNA and cell-free maternal DNA prior to the detecting. In some embodiments, the detecting comprises sequencing. In some embodiments, the sequencing comprises next-generation sequencing. In some embodiments, the liquid sample comprises whole blood. In some embodiments, the liquid sample comprises urine. In some embodiments, the liquid sample comprises cerebrospinal fluid. In some embodiments, the method comprises filtering the liquid sample prior to the detecting. In some embodiments, the cell-free fetal DNA and cell-free maternal DNA is from plasma, wherein the plasma is derived from the whole blood.

In some embodiments, disclosed herein are methods of determining a presence or absence of a condition, or a likelihood of a condition, the method comprising: (a) selectively stabilizing protein from a sample from a subject on a solid matrix; (b) analyzing the stabilized protein; and (c) determining a presence or absence of a condition, or a likelihood of a condition, based on the analyzing. In some embodiments, the condition is a fetal aneuploidy, and the subject is pregnant or suspected of being pregnant. In some embodiments, the solid matrix comprises melezitose. In some embodiments, the protein comprises alpha-fetoprotein (AFP), pregnancy associated plasma protein A (PAPP-A), human chorionic gonadotropin (hCG), unconjugated estriol (uE3), or dimeric inhibin A (DIA). In some embodiments, the solid matrix is configured to reduce protein conformational changes. In some embodiments, the condition comprises pre-eclampsia or eclampsia. In some embodiments, the condition comprises gestational diabetes.

### BRIEF DESCRIPTION OF THE DRAWINGS

Features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A, FIG. 1B and FIG. 1C illustrate different non-planar structures for a matrix provided herein.

FIG. 2 illustrates a jelly-roll or spiral log shape or a matrix provided herein.

FIG. 3A and FIG. 3B illustrate different high surface area textures for a matrix provided herein.

FIG. 4 illustrates an example method for collecting sample on matrix.

FIG. 5 illustrates an example embodiment of a corrugated sheet as provided herein.

FIG. 6 illustrates an example embodiment of a corrugated spiral or spiral provided herein.

FIG. 7 illustrates an example embodiment of a paper roll or spiral provided herein.

FIG. 8 is a flow chart describing an exemplary method for performing a reaction using a reagent stabilization matrix.

FIG. 9 is a flow chart describing an exemplary method for analyte concentration.

FIGS. 10A, 10B, and 10C illustrate different sample acquisition components and piercing elements.

FIG. 11 illustrates an embodiment of a sample acquisition component.

FIG. 12 is a flow diagram depicting a method for extracting a blood sample using a tourniquet.

FIGS. 13A and 13B illustrate an embodiment of the separation component.

FIG. 14 illustrates another embodiment of the sample separation component.

FIG. 15 illustrates a sample stabilization component with a stabilization matrix.

FIG. 16 is a non-limiting list of tests that can be conducted on the sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Overview

In one aspect, provided herein are matrices for storing, preserving, or stabilizing one or more components (e.g., nucleic acids and proteins), where the matrices have a high surface area per unit volume, e.g., greater than or equal to 0.14 mm⁻¹. Surface area per unit volume can be calculated by taking the area of a 250 μl blood spot and dividing by the volume of occupied by the matrix. In some instances the matrix can have a surface are per unit volume of greater than 0.14 mm⁻¹ (e.g. greater than 0.25 mm⁻¹, 0.5 mm⁻¹ 0.75 mm⁻¹, 1.0 mm⁻¹, 1.5 mm⁻¹, 3 mm⁻¹, 5 mm⁻¹, or 10 mm⁻¹). A matrix with a high surface area per unit volume can comprise one or more dried reagents for selectively stabilizing one or more components, e.g, one or more components of a bio-sample, e.g., nucleic acids, metabolites, and proteins. A sample, e.g., bio-sample can be applied to the matrix with a high surface area per unit volume. Upon application, the sample can rehydrate the dried reagents for selectively stabilizing one or more components.

A matrix with a high surface area per unit volume (e.g., a surface area per unit volume greater than 0.14 mm⁻¹) can enable higher levels of buffer or reagent to be deposited on the surface per unit volume than a matrix with a low surface area per unit volume. For example, the matrix with a high surface area per unit volume, e.g., can be used to absorb, adsorb, or take-up, at least 250 μL, 500 μL, 1 mL, 5 mL, 10 mL, 25 mL, 50 mL, 100 mL, 500 mL, or 1 L of liquid. A matrix with a high surface area per unit volume can provide more contact points for sample exposure than a matrix with a low surface area per unit volume (e.g., a matrix with a surface area per unit volume of less than 0.14 mm⁻¹). More points of contact can enable the sample to be stabilized more homogeneously and quickly on the matrix. A sample on a matrix with a high surface area per unit volume can dry more quickly than a sample on a matrix with a low surface area per unit volume, e.g., because of increased exposure of sample to air, e.g., air dried by a desiccant. In some embodiments, a method for treating a sample can comprise a step wherein the sample is left to dry for about 20 to about 30 minutes.

A matrix for storing, preserving, or stabilizing one or more components (e.g., nucleic acids, metabolites, or proteins), where the matrix has a high surface area per unit volume, can come in a variety of shapes and configurations. Shapes can include: a spiral roll, corrugated sheets (which can be arranged into a compressible structure like the collapsible regions of a fan or an accordion), an expandable spiral, granules, and shredded portions.

The compositions and methods described herein can improve the storage and handling of reagents and simplify sample preparation. The present disclosure describes compositions and methods for stabilizing protein and nucleic acid sample preparation reagents. The compositions and methods described herein can provide reagent stabilization at ambient temperatures and dry conditions and simplify sample preparation procedures. The compositions and methods herein can provide approaches for initiating various reactions, including single strand synthesis, fragmentation, amplification, ligation, end repair, among others so that biological sample preparation can be completed with reduced effort by lab technicians.

In another aspect, compositions and methods described herein relate to a substantially dry solid matrix. The substantially dry solid support matrix can be configured to stabilize, e.g., selectively stabilize, proteins, metabolites, and nucleic acids, and the dry solid support matrix can have a sample preparation reagent stabilized therein. The substantially dry solid support matrix for stabilization of sample preparation reagents can include a solid support matrix and one or more sample preparation reagents.

In another aspect, the compositions and methods described herein are directed to concentrating a sample analyte. A sample can be applied to a solid support matrix, e.g., a dry solid support matrix, and an analyte in the sample can be retained within or on the surface of the solid support matrix. The sample applied to the solid support matrix can have a volume, e.g., a first volume, e.g., 1 mL. The solid support matrix can be configured to stabilize, e.g., selectively stabilize, the analyte. The sample, including the analyte, can be dried on the solid support matrix. The solid support matrix comprising the analyte can be washed, e.g., using a wash buffer. The wash buffer can remove components that do not include the analyte. Following washing, at least 50%, 60%, 70%, 80%, 90%, 95%, 99%, or 100% of the analyte applied to the matrix can remain within or on the solid support matrix. An elution buffer can be used to elute the analyte from the solid support matrix. The elution buffer applied to the solid support matrix can have a volume less than the volume of the sample (the first volume). The analyte within or on the solid support matrix can be eluted in the elution buffer. At least 10%, 20%, 30%, 40%, 50%, 60%, 70%, 80%, 90%, 95%, 99%, or 100% of the analyte on the solid support matrix can be eluted in the elution buffer. The concentration of the analyte in the elution buffer can be greater than the concentration of the analyte in the sample. The analyte can be concentrated in the elution buffer at least 2, 5, 10, 25, 50, 100, 500, 1000, 5000, 10,000, 50,000, 100,000, 500,000, or 1,000,000 fold relative to the concentration of the analyte in the sample.

A first step towards improving the availability of bio-sample testing and diagnostic testing can involve the development of systems and methods that enable quick sample collection, stabilization and preservation of biological components such as DNA, RNA, proteins, or combinations thereof. Once stabilized, such bio components can be transferred to laboratories that can perform one or more biological assays on the bio components. Easier sample collection can be particularly beneficial for diagnosing clinical conditions. There can also be a benefit to having a subject collect, prepare, and optionally analyze his or her own blood samples without the assistance of a medical practitioner or access to a medical facility or to have untrained individuals collect and stabilize bio samples from a subject. Three components can be used in this regard; first, tools for collecting biological samples, such as blood; second, systems, devices and methods to provide simple, user-friendly mechanisms for separating, stabilizing and/or storing collected samples or sample components; and third, compatible approaches for analyzing the samples provided through these methods.

In some cases, the diagnostic value of a laboratory test is only as good as the quality of the sample. Technologies described herein can be user-friendly and effective enough that acquisition, collection, optional separation and stabilization of the sample can be performed by trained and un-trained end users. Disclosed within are devices, systems and methods that overcome current limitations by addressing some of the aforementioned issues.

The devices, systems and methods disclosed herein, combine easier sample collection systems with user friendly sample collection, optional separation, stabilization and storage of samples. Furthermore, the devices, systems, and methods incorporated into this system can provide the approaches and tools for laboratories to more easily receive, prepare and analyze samples. Additionally, the present application discloses methods for using the disclosed devices and systems to detect and diagnose medical conditions.

Collectively this application provides a compatible set of systems, devices, methods and applications that can enable samples to be easily collected, stored, pre-treated and prepared for analysis so that sample detection can be accomplished with reduced effort on behalf of the user, patient or sample provider. The disclosed devices, systems and methods can reduce the burdens of diagnostic testing by simplifying the process of collecting, optionally separating, and stabilizing samples, e.g., bio-samples.

Simplifying the process for blood sample collection can involve devices, systems and methods that separate sample components. Current methods for collecting venous blood can rely on one or more dedicated medical professionals to oversee every step of the blood collection process; from collecting the samples(s) to post-collection procedures which can include separation steps including centrifugation, followed by labeling and cold storage to stabilize samples until they are transferred to a laboratory for testing. Addressing these steps can require development and integration of novel methods for acquiring, as well as separating, stabilizing and storing samples.

The systems and methods described herein can be applied to any biological samples from any organism including human. Bio-samples obtained from an organism can be blood, serum, plasma, synovial fluid, urine, tissue or lymph fluids. They can contain whole cells, lysed cells, plasma, red blood cells, skin cells, non-nucleic acids (e.g. proteins), nucleic acids (e.g. DNA, RNA, maternal DNA, maternal RNA), circulating nucleic acids (e.g. cell-free nucleic acids, cell-free DNA/cfDNA, cell-free RNA/cfRNA), circulating tumor DNA/ctDNA, cell-free fetal DNA/cffDNA). Provided herein are methods for downstream analysis of cell-free and/or circulating nucleic acids, e.g., fetal abnormality detection or cancer detection, diagnosis, or monitoring. Several embodiments can disclose methods, devices, and systems for collecting blood samples; however, the systems and methods disclosed herein are not intended to be limited to obtaining a bio-sample from an organism. For example, disclosed embodiments can be used on samples obtained from the environment. Non-limiting examples of environmental samples include water, soil and air samples.

For the purposes of describing the devices, methods, systems, and kits disclosed herein, any individual that uses the devices, methods, systems, or kits to collect a sample can be referred to as the “end user”. The individual, organism, or environment from which a sample is derived can be referred to as the “donor” or “subject”. Once the sample is collected it can be deployed to another facility for testing. At the facility the sample can undergo treatment steps that are selected for based on the devices, systems, methods or kits that were used.

### Matrix

A solid support matrix can be prepared according to the methods of either U.S. Pat. No. 9,040,679 or US Publication No. 20130323723, both of which are incorporated by reference herein in their entirety.

A matrix can comprise one or more different components. The components can be kept in a substantially dry state of less than 10 wt % hydration, such that the solid support matrix comprises less than 10% water, by weight. Examples of a solid support matrix include a natural material, a synthetic material, or a naturally occurring material that is synthetically modified. Components can comprise reagents, buffers, denaturants, inhibitors, reducing reagents, UV protectants, branched carbohydrates, polymers, or other components. The components can be kept in a substantially dry state. A matrix can be solid, gelatinous, fibrous, or porous. Examples of components that can be used in a matrix include a natural material, a synthetic material, or a naturally occurring material that is synthetically modified. The matrix can be selected from the group consisting of paper, glass microfiber and membrane. The matrix can comprise cellulose, nitrocellulose, modified porous nitrocellulose or cellulose based materials, polyethyleneglycol-modified nitrocellulose, a cellulose acetate membrane, a nitrocellulose mixed ester membrane, a glass fiber, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene) membrane, glass fiber membranes, quartz fiber membranes or combinations thereof. Suitable materials that can act as matrix include cellulose, cellulose acetate, nitrocellulose, carboxymethylcellulose, quartz fiber, hydrophilic polymers, polytetrafluroethylene, fiberglass and porous ceramics. Hydrophilic polymers can be polyester, polyamide or carbohydrate polymers. The matrix can comprise paper, for example a cellulose paper including a 903 Neonatal STD card. The matrix can comprise a membrane selected from the group consisting of polyester, polyether sulfone (PES), polyamide (Nylon), polypropylene, polytetrafluoroethylene (PTFE), polycarbonate, and aluminium oxide. Examples of the matrix can also include porous materials, Whatman FTA™ card, cellulose card, or combinations thereof.

In some instances, the substrate can comprise a dry solid matrix comprised of cellulose. A cellulose-based dry solid support matrix can be devoid of any detergent. In some cases, cellulose-based dry solid matrix is not be impregnated with any reagent. Cellulose-based dry solid matrix can be impregnated with a chaotropic salt. Examples of chaotropic salt include, but are not limited to, guanidine thiocyanate, guanidine chloride, guanidine hydrochloride, guanidine isothiocyanate, sodium thiocyanate, and sodium iodide. In some embodiments, a cellulose-based dry solid support matrix is FTA™ Elute (GE Healthcare). In some embodiments, a cellulose-based dry solid support matrix is FTA™ Elute (GE Healthcare). Examples of the aforementioned sample solid support matrix components are disclosed, e.g., in US Publication Nos. 20130289265 and 20130289257.

The substrate or matrix can comprise one or more dried reagents impregnated therein. In some instances a substantially dry state can be less than or equal to 20%, 15%, 10%, or 5% weight water. The dried reagents can comprise protein stabilizing reagents, nucleic acid stabilizing reagents, cell-lysis reagents or combinations thereof. In some embodiments, the protein stabilizing reagents can include trisaccharides, e.g. melezitose, raffinose, maltotriulose, isomaltotriose, nigerotriose, maltotriose, or ketose. In one embodiment, the substrate is disposed on a substrate frame. Non-limiting examples of the sample substrate can include a porous sample substrate, Whatman FTA™ card, cellulose card, or combinations thereof. In some embodiments, the matrix can include at least one stabilizing reagent that preserves at least one biological sample analyte for transport or storage. Examples of suitable reagents for storage media can include one or more of a weak base, a chelating agent and optionally, uric acid or a urate salt or simply the addition of a chaotropic salt, alone or in combination with a surfactant.

A matrix can be devoid of any detergent, or impregnated with any reagent. A matrix can be impregnated with a chaotropic salt. Examples of chaotropic salt include guanidine thiocyanate, guanidine chloride, guanidine hydrochloride, guanidine isothiocyanate, sodium thiocyanate, and sodium iodide. In some embodiments, the matrix can comprise FTA™ Elute (GE Healthcare). Examples of the aforementioned reagents are described, e.g., in US Publication Nos. 20130289265 and 20130289257.

The matrix can be configured to enhance recovery of components that are present in low concentrations on the matrix. The matrix can comprise at least one surface coated with a chemical mixture that enhances recovery of a biological material from the surface of the matrix. The chemical mixture can comprise components selected from the group consisting of vinyl polymer and non-ionic detergent, vinyl polymer and protein, non-ionic synthetic polymer and non-ionic detergent, non-ionic synthetic polymer and protein, polyethylenemine (PEI) and non-ionic detergent, non-ionic detergent and protein, and polyethylenemine (PEI) and protein.

The matrix can be configured to concentrate components within regions of the matrix. A matrix can be configured to enable preferential migration of nucleic acid, proteins, or metabolites specific distances within the matrix.

The matrix can be impregnated with reagents that enable analyte manipulation, including, but not limited to fragmentation, tagging, amplification, circularization, and ligation.

Also provided herein are methods for recovering a biological material from a matrix comprising the steps of i) contacting a surface of a matrix described herein with a sample containing, e.g., a biological material; ii) drying the sample on the surface of the matrix; iii) storing the matrix; and iv) extracting the biological material from the surface of the matrix. In other aspects, step iii) comprises storing the matrix, e.g., paper support, at a temperature in the range of about 4 to about 40° C. The matrix, e.g., paper support, can be stored at a lower temperature depending on the thermal stability of the biological material.

The matrix and methods of making the matrix can comprise coating at least one surface of the matrix with a solution of a chemical mixture that enhances the recovery of a biological material from the surface. Examples of components or chemical mixtures that can coat, or be used to coat the matrix can include a chemical mixture comprising one or more components selected from the group consisting of: polyvinyl pyrrolidone (PVP) and Tween 20, polyvinyl pyrrolidone (PVP) and albumin, Tween 20 and albumin, poly-2-ethyl-2-oxazoline (PEOX) and Tween 20, poly-2-ethyl-2-oxazoline PEOX and albumin, polyethylenemine (PEI) and Tween 20, and polyethylen-emine (PEI) and albumin. Any of the coatings described above can be used for enhancing the recovery of a biological material from a matrix described herein. Examples of these coatings are disclosed, e.g., in US Publ. Nos. US20130323723 and US2013330750, the entireties of which are herein incorporated by reference.

The matrix can be configured to selectively stabilizing a nucleic acid or a protein. In some embodiments, the nucleic acid is an RNA, a DNA, or fragments or combinations thereof. In some embodiments, a matrix configured to selectively stabilize RNA is an RNA stabilization matrix (RSM). In some embodiments, a matrix configured to selectively stabilize a protein is a protein stabilization matrix (PSM).

Reagents can be impregnated and stored in a dry state on a matrix. In some embodiments, a matrix configured to selectively stabilizing nucleic acids can comprise at least one protein denaturant and at least one acid or acid-titrated buffer reagent impregnated and stored in a dry state. Dried reagents can be optionally rehydrated, e.g., by the addition of buffer, water or sample. The matrix can further comprise a weak or strong protein denaturant. In certain aspects the solid matrix is a porous cellulose-based paper such as the commercially available 903, 31-ETF, or FTA Elute™. Performance of this method can permit the storage of nucleic acids, e.g., RNA which can be an unstable biomolecule to store, in a dry format (e.g., on a solid matrix) under ambient temperatures. The matrix can be configured such that rehydration of the matrix provides an acidic pH. In some embodiments, the RNA quality is determined by capillary electrophoresis of the extracted RNA through a bioanalyzer.

The matrix can be configured to selectively stabilize sample preparation reagents wherein the reagent can comprise protein (e.g. one or more enzymes) and/or nucleic acids (e.g primers). The matrix configured to stabilize protein and nucleic acids can comprise an oligosaccharide under a substantially dry state. In some embodiments, the oligosaccharide is a trisaccharides. The oligosaccharide can be selected from melezitose, raffinose, maltotriulose, isomaltotriose, nigerotriose, maltotriose, ketose, cyclodextrin, trehalose or combinations thereof. In some embodiments, the oligosaccharide is melezitose. Melezitose can be a non-reducing trisaccharide sugar, having a molecular weight of 504.44 g/mol. In some embodiments, the matrix can comprise melezitose. In some embodiments, the concentration of the melezitose is any amount less than 30%. The matrix can comprise melezitose under a substantially dry state. In some embodiments, melezitose can have less than 2% of water content. In the matrix, the concentration of the melezitose can be in a range of about 10% to about 30%. The concentration of melezitose can be 15%. In some embodiments, methods of manufacturing can comprise exposing a sample to liquid melezitose, wherein a concentration of the melezitose can be any amount less than 30%. The melezitose can be impregnated in the matrix. In some embodiments, the impregnated melezitose concentration in the matrix is between about 10 to about 30%. In some other embodiments, 15% melezitose is impregnated into the matrix. In some embodiments, the matrix is passively coated or covalently-modified with melezitose. In some other embodiments, the matrix is coated with a 15% solution of melezitose. The matrix can comprise additional components to stabilize protein and/or nucleic acids, including various stabilization molecules. A non-limiting example of a stabilization molecule is validamycin. In some examples, the matrix is further impregnated with one or more reagents, such as lysis reagents, buffer reagents or reducing agents. In some embodiments, the impregnated reagents comprise cell lytic reagents, biomolecule stabilizing reagents such as protein-stabilizing reagents, protein storage chemicals and combinations thereof impregnated therein under a substantially dry state. Examples of the components described above and other embodiments are outlined, e.g, in U.S. Patent Publication No. US20140234942, the entirety of which is incorporated by reference.

The matrix can comprise a buffer reagent. A buffer reagent can be impregnated into the matrix. Buffers can stabilize sample preparation reagents and/or various sample components. The matrix can further include at least one buffer disposed on or impregnated within the matrix, wherein the matrix is substantially dry with a water content of less than 2%. The buffer can be an acid-titrated buffer reagent that generates a pH in a range from about 3 to about 6, or about 2 to about 7. The matrix can contain any one of the following: 2-Amino-2-hydroxymethyl-propane-1,3-diol (Tris), 2-(N-morpholino) ethanesulfonic acid (MES), 3-(N-morpholino) propanesulfonic acid (MOPS), citrate buffers, 4-(2-hydroxyethyl)-1-piperazineethanesulfonic acid (HEPES), phosphate buffers or combinations thereof, or Tris-Hydrochloride (TrisHCl). The matrix can be configured to yield a solution upon rehydration comprising about 20 to about 70 mM Tris-HCl and about 5 to about 30 mM MgCl₂. The amount of various dehydrated buffer reagents impregnated into a matrix can be configured for stabilizing sample preparation reagent(s).

The matrix can comprise a reagent or compound that minimizes nuclease activity, e.g., a nuclease inhibitor. Examples of nuclease inhibitors include RNase inhibitor, compounds able to alter pH such as mineral acids or bases such as HCl, NaOH, HNO₃, KOH, H₂SO₄, or combinations thereof; denaturants including urea, guanidine hydrochloride, guanidinium thiocyanate, a one metal thiocyanate salt that is not guanidinium thiocyanate (GuSCN) beta-mercaptoethanol, dithiothreitol; inorganic salts including lithium bromide, potassium thiocyanate, sodium iodide, or detergents including sodium dodecyl sulfate (SDS).

The matrix can comprise a reagent or compound that minimizes or inhibits protease activity, e.g., a protease inhibitor. A protease inhibitor can be synthetic or naturally-occurring (e.g., a naturally-occurring peptide or protein). Examples of protease inhibitors include aprotinin, bestatin, chymostatin, leupeptin, alpha-2-macroglobulin, pepstatin, phenylmethanesulfonyl fluoride, N-ethylmaleimide, ethylenediaminetetraacetid acid, antithrombin, or combinations thereof. In one example, protease inhibitors enhance the stability of the proteins by inhibiting proteases or peptidases in a sample.

In some embodiments, the solid matrix is configured to reduce protein conformational changes. In some embodiments, the solid matrix comprises a reagent or compound to reduce protein conformation changes. In some embodiments, the reagent or compound that reduces protein conformation changes is part of a buffer. In some embodiments, the reagent or compound that reduces protein conformation changes comprises a protease inhibitor, glycerol, bovine serum albumin (BSA), a chelating agent, a reducing agent, or a combination thereof. In some embodiments, any suitable reagent or compound for reducing protein conformation changes is used.

The solid support matrix can comprise one or more free radical scavengers. The solid support matrix can comprise a UV protectant or a free-radical trap. Exemplary UV protectants include hydroquinone monomethyl ether (MEHQ), hydroquinone (HQ), toluhydroquinone (THQ), and ascorbic acid. In certain aspects, the free-radical trap can be MEHQ. The solid support matrix can also comprise oxygen scavengers, e.g. ferrous carbonate and metal halides. Other oxygen scavengers can include ascorbate, sodium hydrogen carbonate and citrus.

The matrix can comprise a cell lysis reagent. Cell lysis reagents can include guanidinium thiocyanate, guanidinium hydrochloride, sodium thiocyanate, potassium thiocyanate, arginine, sodium dodecyl sulfate (SDS), urea or a combination thereof. Cell lysis reagents can include detergents, wherein exemplary detergents can be categorized as ionic detergents, non-ionic detergents, or zwitterionic detergents. The ionic detergents can comprise anionic detergent such as, sodium dodecylsulphate (SDS) or cationic detergent, such as ethyl trimethyl ammonium bromide. Examples of non-ionic detergent for cell lysis include TritonX-100, NP-40, Brij 35, Tween 20, Octyl glucoside, Octyl thioglucoside or digitonin. Some zwitterionic detergents can comprise 3-[(3-Cholamidopropyl)dimethylammonio]-1-propanesulfonate (CHAPS) and 3-[(3-Cholamidopropyl)dimethylammonio]-2-hydroxy-1-propanesulfonate (CHAPSO). The cell lysis reagent can comprise a thiocyanate salt. One or more embodiments of the matrix comprises a thiocyanate salt impregnated in a dry state. Exemplary thiocyanate salts include guanidinium thiocyanate, sodium thiocyanate, potassium thiocyanate or combinations thereof. In some embodiments, the cell lysis reagent is selected from guanidinium thiocyanate, sodium thiocyanate, sodium dodecyl sulfate (SDS) or combinations thereof.

A matrix can comprise a reducing agent. Reducing agents can include dithiothreitol (DTT), 2-mercaptoethanol (2-ME), tris(2-carboxyethyl)phosphine (TCEP) and combinations thereof. Reducing agents can further comprise oxygen scavengers. Oxygen scavengers or reducing agents can comprise ferrous carbonate and metal halides.

A matrix can comprise a chelating agent. Chelating agents can include ethylenediaminetetraacetic acid (EDTA), citric acid, ethylene glycol tetraacetic acid (EGTA), or combinations thereof.

The matrix can be configured to provide an acidic pH upon hydration and/or preserve nucleic acids in a substantially dry state at ambient temperature. The matrix can be configured to provide a pH between about 2 and about 7 upon hydration. The matrix can be configured to provide a pH between about 3 and about 6 upon hydration.

A matrix for selectively stabilizing nucleic acids can comprise any combination of reagents including a protein denaturant, a reducing agent, buffer, a free-radical trap, a chaotropic agent, a detergent, or an RNase inhibitor in the matrix in a dried format. The RNase inhibitor can comprise a triphosphate salt, pyrophosphate salt an acid, or an acid-titrated buffer reagent. The matrix can further be impregnated with one or more reagents including enzyme inhibitors, free-radical scavengers, or chelating agents. The matrix can comprise a protein denaturant, a reducing agent, a buffer, and optionally a free-radical trap or RNase inhibitor.

The matrix can further comprise a weak or strong protein denaturant. In certain aspects the solid matrix is a porous cellulose-based paper such as the commercially available 903, 31-ETF, or FTA Elute™. Performance of this method permits the storage of nucleic acids, particularly RNA which is widely known to be an unstable biomolecule to store, in a dry format (e.g., on a solid matrix) under ambient temperatures. The matrix can be configured such that rehydration of the matrix provides an acidic pH. In some embodiments, the RNA quality is determined by capillary electrophoresis of the extracted RNA through a bioanalyzer.

The matrix can permit prolonged storage of one or more bio-components or reagents comprising nucleic acids (e.g., RNA, DNA) in a dry format under ambient conditions. In other aspects, a matrix for ambient extraction and storage of nucleic acids (e.g., RNA, DNA) from a sample comprises a thiocyanate salt, a reducing agent, a buffer, and optionally a free-radical trap or RNase inhibitor in a dried format. A matrix for extraction and storage of nucleic acids (e.g., RNA, DNA) from a sample can comprise at least one metal thiocyanate salt, wherein at least one metal thiocyanate salt is not guanidinium thiocyanate (GuSCN), a reducing agent, a buffer, and optionally a free-radical trap or RNase inhibitor. The matrix can comprise nucleic acids (e.g., RNA, DNA) in a dry format, and the nucleic acids can be subjected to a process to release the nucleic acids from the matrix in an intact format that is suitable for further analyses of the collected nucleic acid samples.

The matrix configured to selectively stabilize nucleic acid sample preparation reagents can permit prolonged dry preservation of nucleic acids from a sample under ambient storage conditions. The matrix can be configured to preserve nucleic acid reagents with varying degrees of integrity. For example, the degree of maintained nucleic acid integrity can vary depending on variables including time temperature and humidity. Nucleic acid or RNA integrity can be characterized with an RNA Integrity Number (RIN). In some embodiments, the RIN is calculated by an algorithmic assessment of the amounts of various RNAs present within the extracted RNA. High-quality cellular nucleic acids can generally exhibit a RIN value approaching 10. The RNA extracted from the matrix can have a RIN value of at least 4, at least 5, at least 6, at least 7, or greater than 7. The RNA extracted from the matrix can have a RIN value ranging from 4 to 10, or alternately, the RIN value can be in a range from 5 to 8. RIN can be determined using software, algorithms, or other tools recognized by one having skill in the art.

In some embodiments, the RIN number is calculated on an analyzer, for example an Agilent 2100 analyzer. The RIN can be calculated from capillary electrophoretic measurements, which separates nucleic acids according to their length by applying an electrophoretic field. In electrophoretic measurements the sample can be run through a gel within a very thin tube, with a detector at the end that detects the small fragments first. Since the small samples pass through first, an electropherogram can be produced with the fragment size related to the time of elution. The RIN can be assigned independent of sample concentration, instrument and analyst. Therefore, a RIN can be a standard means of measuring RNA integrity.

The RIN can be a software tool that can use an entire electrophoretic trace of an RNA sample to determine sample integrity. The RIN can be calculated using features of the electrophoretic trace from an RNA sample. For a eukaryotic sample, the features can include 1) total RNA ratio, 2) height of 28S peak, 3) fast area ratio, and 4) marker height. Total RNA ratio can be calculated by taking the ratio of the area under the 18S and 28S rRNA peaks from the electropherogram. The height of the 28S peak can be taken directly from the electropherogram. The fast area ratio can be the area between the 18S and 5S rRNA peaks on an electropherogram. A marker height can be calculated from the height of a peak for a given marker.

High-quality cellular RNA can exhibit a RIN value approaching 10. In some embodiments, the RNA extracted from a matrix has a RIN value of at least 4. In some embodiments, the matrix provides for ambient extraction and stabilization of a bio-sample and produces intact, high quality RNA with a RIN value in a range from 4 to 10, or in some embodiments, the RIN value is in a range from about 5 to about 8. The matrix can be a porous non-dissolvable dry material configured to provide a pH between about 2 and about 7 upon hydration for extracting RNA. The matrix can stabilize the extracted RNA with an RNA Integrity Number (RIN) of at least 4. The matrix can stabilize the extracted RNA, such that the RNA will retain an RNA Integrity Number (RIN) of at least 4 after greater than or equal to one day, 10 days, 25 days, 45 days, or 60 days of dry storage under ambient conditions, or standard temperature and pressure (STP) conditions.

The matrix can stabilize nucleic acids such that eluted nucleic acids have a RIN value greater than 4, even when nucleic acids are stabilized under a variety of conditions including varying lengths of time, at various temperatures and various relative humidity levels. Nucleic acids can be stabilized for 2 days or more. Nucleic acids stabilized for about 2 days to about 30 days can have a RIN value of 4 or greater. Under various temperatures and relative humidity levels described herein, the matrix can stabilize RNA such that there is a RIN value of 4 or greater.

The matrix can stabilize nucleic acids such that eluted nucleic acids that have a RIN value of 4 or greater when stored at room temperature, in ambient conditions. RNA eluted from a matrix can have a RIN value of 4 or greater when stored in a substantially dry environment, at about 5% relative humidity, at about 10% relative humidity, at about 20% relative humidity, at about 30% relative humidity, at about 40% relative humidity, at about 50% relative humidity, at about 60% relative humidity, or at about 70% relative humidity. RNA eluted from a matrix can have a RIN value of 4 or greater when stored a wide range of temperatures, including room temperature, ambient temperatures, and temperatures ranging from about 15° C. to about 60° C. RNA eluted from a matrix can have a RIN value of 4 or greater at temperatures of about 15° C., about 20° C., about 25° C., about 30° C., about 35° C., about 40° C., about 45° C., about 50° C., about 55° C., or about 60° C. RNA eluted from a matrix can have a RIN value of 4 or greater at temperatures of less than about 15° C., about 20° C., about 25° C., about 30° C., about 35° C., about 40° C., about 45° C., about 50° C., about 55° C., or about 60° C.

Methods for extracting and storing nucleic acids from a sample can comprise steps of applying the sample to a matrix comprising a protein denaturant and an acid or acid titrated buffer reagent; generating an acidic pH upon hydration for extraction of nucleic acids from the sample; drying the matrix comprising the stabilized nucleic acids from other components on the matrix; and storing the nucleic acids on the matrix in a substantially dry state at ambient temperature. Examples of the aforementioned sample stabilization components can be found in US Pub. No. US20130338351.

Other solid matrices for stabilizing metabolites or proteins can comprise a solid paper based matrix comprised of cellulose fibers and/or glass fibers and a hydrophilic or water soluble branched carbohydrate polymer. Surface weight of the solid paper based matrix can be about 40 to about 800 g/m². Hydrophilic or water soluble branched carbohydrate polymer can be about 4 to about 30 wt % of the solid paper based matrix. The hydrophilic or water soluble branched carbohydrate polymer can have average molecular weight of about 15 to about 800 kDa, such as about 20 to about 500 kDa. The branched carbohydrate polymer can include a dextran. Dextrans can be branched a (16)-linked glucans. The branched carbohydrate polymer can comprise a copolymer of a mono- or disaccharide with a bifunctional epoxide reagent. Such polymers can be highly branched due to the multitude of reactive hydroxyl groups on each mono/disaccharide. Depending on the reaction conditions used, the degree of branching can be from about 0.2 up to almost 1. The content of water extractables in the solid paper based matrix can be 0 to about 25 wt %, such as about 0.1 to about 5 wt % or about 3 to about 20 wt %. Very low amounts of extractables can be achieved when the carbohydrate polymer is covalently coupled to the paper fibers and/or crosslinked to itself. In some embodiments, the solid paper based matrix comprises about 5 to about 300 micromole/g, such as about 5 to about 50, about 5 to about 100 or about 50 to about 300 μmol/g negatively or positively charged groups. Negatively charged groups can be e.g., carboxylate groups, sulfonate groups or sulfate groups, while positively charged groups can be, e.g., amine or quaternary ammonium groups. The presence of these groups can improve the protective effect of the branched carbohydrate polymer.

Methods for removing sample can comprise a step of applying a sample to a matrix, and storing the dried matrix, e.g., paper with a biological sample for at least one day, at least one week, at least one month, or at least one year. In some embodiments the method comprises a step of extracting at least one protein from the matrix, e.g., paper, after storage, and then analyzing the protein. The extraction can be performed by, e.g., punching out small parts of the matrix, e.g., paper, with dried sample and immersing these in an aqueous liquid. In some embodiments the protein is analyzed by an immunoassay, by mass spectrometry or an enzyme activity assay. Examples of the matrix disclosed herein are described, e.g., in US Pub. No. US20140302521.

The matrix can be dry and can be stored under ambient conditions. Storage under such conditions can obviate the need for a cold chain. The matrix can have water content of less than 2%. The matrix can be stored in ambient conditions. A non-limiting example of ambient conditions is temperature ranging from −20° C. to 60° C. or room temperature and relative humidity ranging from about 35% to about 75%. “About” can refer to an amount 10% greater than or less than a recited quantity. Room temperature can be about 15° C. to about 35° C. The matrix can be stored in a substantially dry environment, at about 5% relative humidity, at about 10% relative humidity, at about 20% relative humidity, at about 30% relative humidity, at about 40% relative humidity, at about 50% relative humidity, at about 60% relative humidity, or at about 70% relative humidity. The matrix can be stored at less than about 5% relative humidity, at less than about 10% relative humidity, at less than about 20% relative humidity, at less than about 30% relative humidity, at less than about 40% relative humidity, at less than about 50% relative humidity, at less than about 60% relative humidity, or at less than about 70% relative humidity. The matrix can be stored at a wide range of temperatures, including room temperature, ambient temperatures. Ambient temperatures can range from about −20° C. to about 60° C. Room temperature can range from about 15° C. to about 35° C. The matrix can be stored at temperatures ranging from about 4° C. to about 60° C. The matrix can stabilize various molecules including sample preparation reagents and biological sample components at temperatures of about 4° C., about 15° C., about 20° C., about 25° C., about 30° C., about 35° C., about 40° C., about 45° C., about 50° C., about 55° C., about 60° C. The matrix can be stored at temperatures of less than about 15° C., less than about 20° C., less than about 25° C., less than about 30° C., less than about 35° C., less than about 40° C., less than about 45° C., less than about 50° C., less than about 55° C., less than about 60° C.

The matrix can comprise at least one dried biological sample, such as a dried blood sample. Blood and other biological materials, e.g., serum, plasma, cell lysate, urine, cerebrospinal fluid, bone marrow, biopsies etc. can be applied to the matrix and dried for storage and subsequent analysis or other use. The dried biological sample can also be a pharmaceutical formulation or a diagnostic reagent, comprising at least one protein or other sensitive biomolecule. In another aspect the matrix can comprise a paper card with one or more sample application areas printed or otherwise indicated on the card. There can be indicator dyes in these areas to show if a non-colored sample has been applied or not. The device can also include a card holder, to e.g., facilitate automatized handling in racks etc., and it can include various forms of sampling features to facilitate the collection of the sample.

In some embodiments, the solid matrix can selectively stabilize blood plasma components. Plasma components can include cell-free DNA, cell-free RNA, protein, hormones, and other metabolites, which can be selectively stabilized on the solid matrix. Plasma components can be isolated from whole blood and stabilized on a solid matrix. A solid matrix can be overlapping with or a component of a variety of different devices and techniques. Plasma components can be separated from whole blood samples using a variety of different devices and techniques. Techniques can include lateral flow assays, vertical flow assays, and centrifugation.

A solid matrix can be integrated with or a component of a variety of plasma separation devices or techniques. A solid matrix can be overlapping with or a component of a variety of different devices, such as a plasma separation membrane for example Vivid™ plasma separation membrane. A solid matrix can partially overlap with plasma separation device such as a plasma separation membrane. Examples of devices and techniques for plasma separation are disclosed in patents or patent publications, herein incorporated by reference, including U.S. Pat. Nos. 6,045,899; 5,906,742; 6,565,782; 7,125,493; 6,939,468; EP 0,946,354; EP 0,846,024; U.S. Pat. Nos. 6,440,306; 6,110,369; 5,979,670; 5,846,422; 6,277,281; EP 1,118,377; EP 0,696,935; EP 1,089,077, US 20130210078, and US 20150031035.

In various devices and techniques, a separation membrane can be used. The separation membrane can be comprised of polycarbonate, glass fiber, or others recognized by one having skill in the art. Membranes can comprise a solid matrix. Membranes can have variable pore sizes. Separation membranes can have pore diameters of about 1 μm, about 2 μm, about 4 μm, about 6 μm, about 8 μm, about 10 μm, about 12 μm, about 14 μm, about 16 μm, about 18 μm, about 20 μm. A separation membrane can have pores with diameters of about 2 μm to about 4 μm. A separation membrane can have pores that are about 2 μm in diameter.

Plasma separation can be implemented for a wide variety of sample volumes. Plasma sample volumes can be variable depending on the application for which a solid matrix is used. Sample volumes can be greater than about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL. Sample volumes can range from about 250 μL to about 500 μL.

Other examples of stabilization matrix or stabilization components that can be used in the devices and methods described include, but are not limited to Gentegra-RNA, Gentegra-DNA (Gentegra, Pleasanton CA), as further illustrated in U.S. Pat. No. 8,951,719; DNA Stable Plus, as further illustrated in U.S. Pat. No. 8,519,125; RNAgard Blood System (Biomatria, San Diego, CA).

### High Surface Area Per Unit Volume

In some instances the matrix can have a surface are per unit volume of greater than 0.14 mm⁻¹ (e.g. greater than 0.25 mm⁻¹, 0.5 mm⁻¹ 0.75 mm⁻¹, 1.0 mm⁻¹, 1.5 mm⁻¹, 3 mm⁻¹, 5 mm⁻¹, or 10 mm⁻¹). Surface area per unit volume can be calculated by taking the area of a 250 μl blood spot and dividing by the volume occupied by the matrix.

In some instances the matrix can be configured to comprise a planar sheet with total dimensional area (length multiplied by width) greater than 176 mm². In some instances the matrix comprises a surface area equivalent to a planar dimensional area greater than 150 mm², greater than 400 mm², greater than 700 mm², greater than 1200 mm², greater than 3200 mm², greater than 5000 mm², greater than 8000 mm², or greater than 1776 mm². A sample, e.g., blood, can be applied to a matrix with any of the aforementioned dimensions.

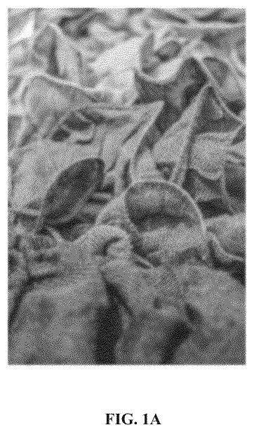

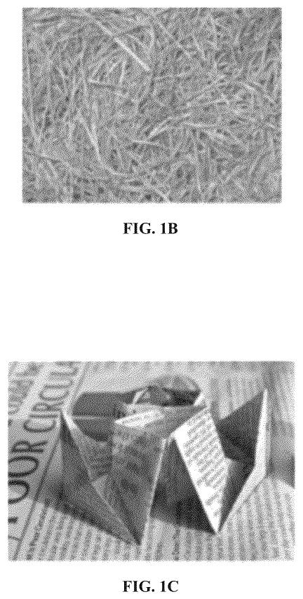

A substrate or matrix can be comprised of one or more layers of material (see e.g., FIG. 1A and FIG. 1B). Layers can be arranged to selectively extract specific bio-sample components. Layers can be oriented horizontally, vertically, stacked, latticed, or interwoven. The substrate can be arranged into a solid matrix. Layers can be arranged to selectively extract specific sample, e.g., bio-sample components. A matrix can comprise a single material, or it can comprise multiple materials.

The sample stabilization systems and devices disclosed herein can comprise solid matrices with high surface areas per unit volume configured to rapidly absorb sample and efficiently selectively stabilize one or more components of the sample. Rapid absorption can occur on a time scale of seconds to minutes. In some examples, absorption can occur at a rate of greater than (faster than) or equal to 100 mL/minute, 10 mL/minute, 1 ml/minute, 0.75 ml/min, 0.5 ml/min, 0.25 ml/min, 0.1 ml/min. A matrix provided herein can absorb a sample, e.g., liquid sample, in about 0.1 mL/min to about 1 mL/min, about 1 mL/min to about 10 mL/min, or about 10 mL/min to about 100 mL/min.

In some embodiments, a system, a method, or a device can comprise a high surface area per unit volume matrix that selectively stabilizes nucleic acids, metabolites, or proteins.

The matrix can have anon-planar structure (FIG. 1C). The non-planar structure can have a length, width, and/or height greater than 1 cm, 5 cm, 10 cm, 100 cm, or 1000 cm, and occupy a large volume (e.g. a volume greater than 10 cm³, 1 cm³, 100 mm³, or 10 mm³). The non-planar structure can a have a length, width, and/or height less than 1000 cm, 100 cm, 10m, 5 cm, or 1 cm.

A non-planar structure can be configured to absorb at least 10 ml volume of blood in a tube with dimensions of at least or about 16 mm×100 mm. In other embodiments, the volume of a blood sample can be between 1 ml and 5 ml, e.g., in a tube with dimensions of at least or about 13×75 mm. In yet additional embodiments, a blood sample volume of less than or equal to 1.5 ml (e.g., about 1.5 ml, 1 ml, 0.5 ml) can be collected. In some instances a matrix can comprise a 3-dimensional structure having a height, width, and length each greater than 13.3 mm. In some instances a matrix can comprise a 3-dimensional structure having a height, width, and length each less than or equal to 13.3 mm. A non-planar matrix can be put in a vessel that can hold a volume of at least 1 mL, 5 mL, 10 mL, 50 mL, 100 mL, 500 mL, or 1000 mL. A non-planar matrix can be put in a vessel that can hold a volume of about 0.1 mL to about 2 mL, about 0.1 mL to about 10 mL, about 0.1 mL to about 100 mL, about 10 mL to about 100 mL, or about 100 mL to about 1000 mL.

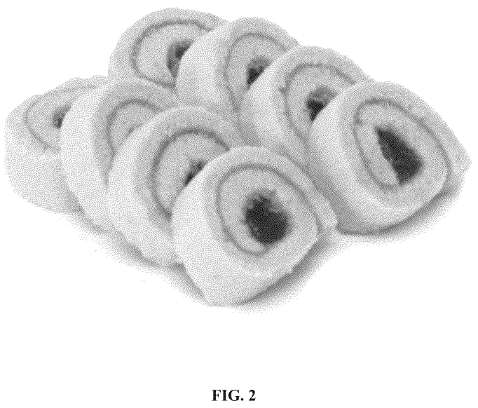

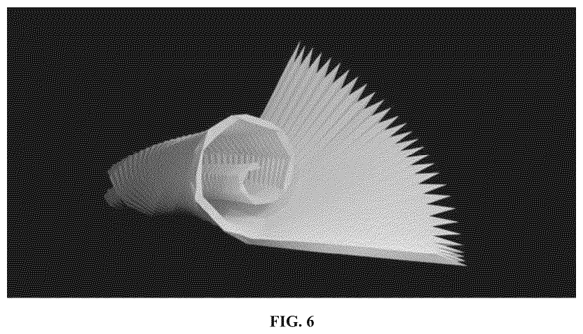

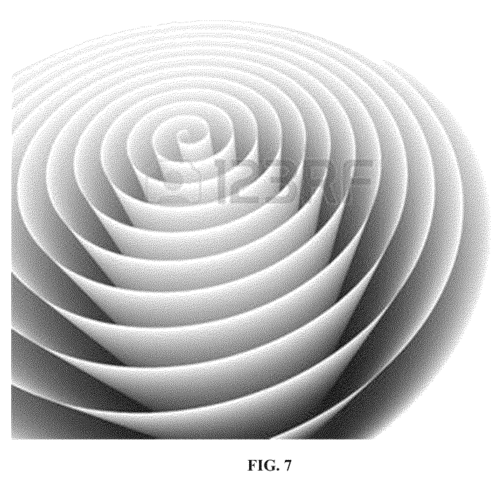

The matrix can comprise anon-planar structure configured to selectively stabilize nucleic acids, metabolites, or proteins. In further embodiments, a matrix can comprise a spiral roll shape. A spiral roll can resemble a log, a rolled cake, or a jelly-roll shape (FIG. 2). In some embodiments the matrix can be porous, the matrix can comprise a material with a plurality of inner channels and pores. A matrix can comprise a single or multiple corrugated sheets (FIG. 5). Corrugated sheets (FIG. 5) can be arranged into a compressible structure like the collapsible regions of a fan or an accordion. Corrugated sheets can form a lattice of interweaved and corrugated layers. Corrugated sheets can be formed into different shapes, including corrugated spirals as shown in FIG. 6. A matrix can be formed into or rolled into shapes, e.g. have a shape similar to a spiral as shown in FIG. 7.

In some instances the shapes can be expandable, e.g. the spiral can be expandable. Expansion can occur laterally, vertically or from all directions. Expansion can occur due to extending the area between the two ends of the spiral or from absorption of sample. A matrix can comprise granules, and granules can have a variable or constant diameter. In some instances a matrix that selectively stabilizes nucleic acids or proteins can comprise one or more dried buffers or reagents and a material configured to absorb a large volume of sample. In some instances, a matrix can absorb greater than or equal to 1000 mL, 100 mL, 10 mL, 8 mL, 6 mL, 4 mL, 2 mL, 1.5 mL, 500 μl, or 250 μl of sample, e.g., blood.

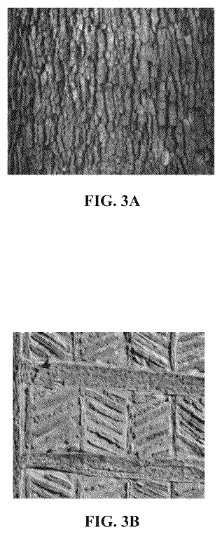

A matrix can comprise a sponge material. In some instances the sponge material can expand upon exposure to sample. In some embodiments, the matrix can be configured to absorb >250 μL of sample. A matrix can comprise a plurality of inner channels and cavities or surface features or microstructure (FIG. 3A and FIG. 3B). In some instances a matrix can comprise a solid foam. In some further instances the foam does not become solid until it has been exposed to sample and dried. In some instances a matrix can comprise one or more corrugated sheets. A matrix can further comprise an expanded spiral or spring. In some instances a matrix can comprise granules. In some instances the granules can be smaller than a grain of sand. In other instances the granules can be smaller than the size of a poppy seed. In further instances the granules can be smaller than the size of a dust particle or a particle of flour. In some instances the matrix can turn into a gel upon exposure to sample. In other instances the matrix can comprise non-spherical fragments, for example, with an average diameter <100 μm, less than 10 μm, less than 100 μm, or less than 1000 μm.

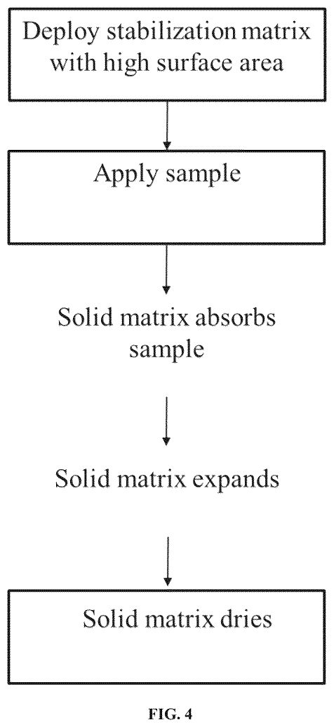

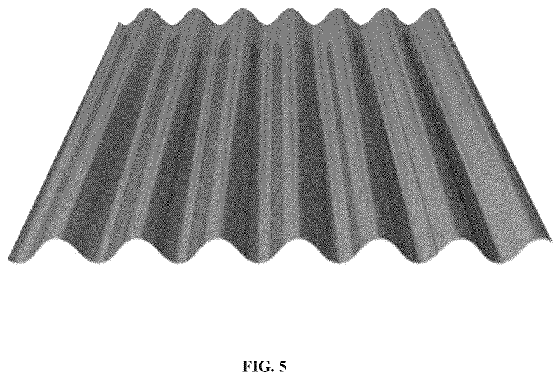

FIG. 4 generally describes an example of how a stabilization matrix with high surface area can be used. A sample can be applied to the stabilization matrix. In some cases, absorption of the sample by the matrix can result in the matrix expanding. The matrix can be allowed to dry.

In some embodiments a matrix can be enclosed or installed into a sample vessel. In further instances, the matrix can be preloaded into a sample vessel. In some instances the matrix with any of the above shapes can fill the bottom of a vessel. In some embodiments, granules of matrix can be deposited in the bottom of a tube for collecting and storing sample. In some embodiments, matrix can be stored in a separate compartment. In some embodiments, a sample vessel can comprise a first compartment for storing a matrix, a second compartment for a sample, and a third compartment for rehydration buffer or reagents. In some embodiments, a sample vessel can be configured for use with a liquid handling robot.

A sample vessel can comprise plastic, glass, or other polymer based material. In some instances, a sample vessel can be opaque, transparent, or dark to protect the sample from exposure to UV or light.

A sample vessel can comprise a vacutainer blood collection tube with a closure that is evacuated to create a vacuum inside the tube. The vacuum inside the tube can facilitate the draw of a volume of blood. In some instances the vacuum in the tube can facilitate the collection of a predetermined volume of blood.

In some instances a sample vessel does not have a pre-established vacuum inside the tube, but can instead comprise a small bulb for creating vacuum upon depression or compression of the bulb.

A sample vessel, or device, comprising a matrix can further comprise desiccant for drying the matrix after absorption of the sample.

A method for stabilizing samples, e.g., bio-samples can comprise the steps of contacting a sample, e.g., bio-sample with any of the previously disclosed matrix structures or components. In further embodiments, a method can be used for stabilizing a protein, metabolite, or nucleic acid from a sample having a defined sample volume. In some embodiments, this method can comprise the steps of contacting a liquid sample with a matrix that selectively stabilizes the protein, metabolite, or the nucleic acid.

In some embodiments, the matrix has a ratio of a volume of sample absorbed per mm² of a matrix surface area. In some embodiments, the ratio of the sample volume to the matrix surface area is at least 75 μL/176 mm² or 0.426 μL/mm². In some embodiments, the ratio of the sample volume to the matrix surface area is at least 0.1 μL/mm², 0.2 μL/mm², 0.3 μL/mm², 0.4 μL/mm², 0.5 μL/mm², 0.6 μL/mm², 0.7 μL/mm², 0.8 μL/mm², 0.9 μL/mm², 1 μL/mm², 2 μL/mm², 3 μL/mm², 4 μL/mm², 5 μL/mm², 6 μL/mm², 7 μL/mm², 8 μL/mm², 9 μL/mm², 10 μL/mm², or 20 μL/mm². In some embodiments, the ratio of the sample volume to the matrix surface area is less than 0.1 μL/mm², 0.2 μL/mm², 0.3 μL/mm², 0.4 μL/mm², 0.5 μL/mm², 0.6 μL/mm², 0.7 μL/mm², 0.8 μL/mm², 0.9 μL/mm², 1 μL/mm², 2 μL/mm², 3 μL/mm², 4 μL/mm², 5 μL/mm², 6 μL/mm², 7 μL/mm², 8 μL/mm², 9 μL/mm², 10 μL/mm², or 20 μL/mm². In some cases, the ratio of sample volume to matrix surface area is about 0.1 μL/mm² to about 1 μL/mm² or about 1 μL/mm² to 10 μL/mm².

In some instances, a method for stabilizing a protein or nucleic acid comprises using a high surface area per unit volume matrix to selectively stabilize one or more components from a liquid sample having a volume of about, greater than, or less than 1000 mL, 100 mL, 10 mL, 1 mL, 500 μl, or 250 μl. Further embodiments can comprise contacting the liquid sample with a matrix that selectively stabilizes the protein or the nucleic acid.

In some instances a method for stabilizing a protein or nucleic acid in a liquid sample can comprise the steps of contacting the liquid sample with a matrix comprising a reagent that selectively stabilizes the protein or the nucleic acid upon contact of the liquid sample with the matrix, e.g., by hydration of the reagent such that stabilization occurs in solution or on the matrix.

The nature of the sample can for example depend upon the source of the material, e.g., biological material. For example, the source can be from a range of biological organisms including, but not limited to, virus, bacterium, plant and animal. The source can be a mammalian or a human subject. For mammalian and human sources, the sample can be selected from the group consisting of tissue, cell, blood, plasma, saliva and urine. In another aspect, the sample is selected from the group consisting of biomolecules, synthetically-derived biomolecules, cellular components and biopharmaceutical drug. In some embodiments, the sample is a solution comprising a nucleic acid, a protein, or a combination. In some embodiments, the nucleic acid, protein, or combination thereof in the solution was purified from an initial sample taken from a biological organism. The sample can be from an archaeological sample, forensic sample, medical sample, sample resulting from a terrorism (bio-terrorism) event, quality-control sample, sample resulting from a natural disaster, or from a security checkpoint (e.g., at an airport or a border between states or countries).

### Sample Preparation Reagents

A solid support matrix described herein can comprise stabilized sample preparation reagents. Various methods can be used to incorporate sample preparation reagents within the solid support matrix. A sample preparation reagent can be impregnated into the solid support matrix. Deposition can further comprise the tagging and/or binding reagents.

A solid support can be impregnated by dipping, wetting, spraying, printing, or saturating the solid support matrix with a solution comprising a sample preparation reagent. The reagent-impregnated solid support matrix can dried using, for example, in the presence of a dessicant, by air-drying, or in an oven, e.g., line oven conveyors. In some instances, oligonucleotide sample preparation reagents can be synthesized directly onto the solid support matrix. The solid support matrix can be impregnated with oligonucleotide sequences or probes using inkjet printing.

The solid support matrix can comprise varying amounts of stabilized, dehydrated sample preparation reagents. The solid support matrix can carry at least about 0.375 microliters (μL) of sample preparation reagent solution per square millimeter (mm) of solid support matrix. The solid support can carry about 0.375 μL to about 0.5 μL of sample preparation reagent solution per square millimeter (mm) of solid support matrix. The solid support matrix can be configured such that the surface area and amount of dehydrated reagent therein results in a sample preparation reagent solution upon hydration.

Sample preparation reagents within a solid support matrix, e.g., stabilized within a solid support matrix, can be selected to initiate a specific reaction. The sample preparation reagent can react with a particular analyte of interest or complete a particular sample preparation step. Examples of sample preparation reactions include single strand extension, fragmentation (e.g., nucleic acid fragmentation, fragmentation with restriction enzymes, chemical fragmentation), tagging (e.g., nucleic acid tagging, incorporation of a modified nucleotide, addition of photoreactive tag), sequencing (e.g. Sanger sequencing, sequencing by synthesis, single molecule real time sequencing), amplification (e.g., nucleic acid amplification, e.g., PCR, AFLP-PCR, ligase chain reaction (LCR)), hybridization (e.g., intramolecular or intermolecular nucleic acid hybridization), ligation (e.g., intramolecular or intermolecular nucleic acid ligation, ligation of two double stranded nucleic acids, ligation of two single-stranded nucleic acids, ligation of adaptors), end repair reaction (e.g., nucleic acid end repair, nonhomologous end-joining, homologous end-joining), adenylation reaction, base excision repair reaction, phosphorylation reaction, an immunohistochemistry reaction, a dephosphorylation reaction, methylation reaction, demethylation reaction, restriction enzyme digestion (e.g., double stranded nucleic acid, e.g., DNA or RNA, or single-stranded nucleic acid, e.g., DNA or RNA; endonuclease or exonuclease), reaction using telomerase, protein cleavage reaction (e.g., using a protease) or bioconjugation reaction fragmentation reaction. A bioconjugation reaction can include a reaction for specifically linking a marker molecule or tag to a biomolecule, e.g. a dye molecule, to a e.g. peptide, protein, antibody.

The solid support matrix can comprises sample preparation reagents, and the sample preparation reagents can comprise research nucleic acid reagents or protein reagents. The sample preparation reagents can be stabilized on the solid support. The sample preparation reagents can be selectively stabilized on the solid support. A solid support matrix can be configured to stabilize both protein sample preparation reagents and a nucleic acid sample preparation reagents. Nucleic acid reagents can be stabilized in a solid support matrix configured to stabilize nucleic acids. Protein reagents can be stabilized in a solid support matrix configured to stabilize proteins.

A solid support matrix can be configured to selectively stabilize sample preparation reagents comprising nucleic acids. A solid support matrix configured to selectively stabilize sample preparation reagents comprising nucleic acids can also comprise a protein denaturant, a reducing agent, or a buffer. A free-radical trap or RNase inhibitor can be incorporated into the solid support matrix in a dry state. The solid support matrix configured to stabilize a sample preparation reagent comprising nucleic acid can also comprise at least one thiocyanate salt, wherein at least one thiocyanate salt is not guanidinium thiocyanate (GuSCN), a reducing agent, a buffer, and optionally a free-radical trap or RNase inhibitor present in the solid support matrix in a dried format. The composition can optionally further comprise an ultraviolet (UV) inhibitor, a free-radical trap, an RNase inhibitor, a chelator, or any combination thereof. The solid support matrix can be configured to provide an acidic pH upon rehydration.

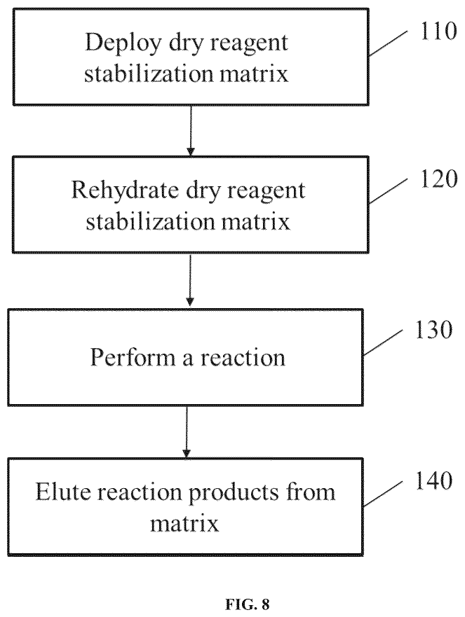

FIG. 8 generally describes an example of how a solid support matrix having sample preparation reagents stabilized therein can be used. A dry solid matrix having sample preparation reagents stabilized therein can be deployed to a user (110). The user can then rehydrate the dry solid support matrix (120), thereby initiating a reaction using at least the reagents stabilized therein (130). After a reaction is carried out to a desired degree, reaction product can then be eluted from the matrix (140).

The solid support matrix can comprise sample preparation nucleic acid reagents that can include various lengths of single or double stranded DNA, RNA, or nucleotides. The sample preparation nucleic acid reagents can be stabilized, e.g., selectively stabilized, on the solid support matrix. Generally speaking, sample preparation nucleic acid reagents can include, but are not limited to, primers, probes, index sequences or tags, and adapters.

Primers can comprise oligodT primers, random primers, universal primers, primers configured for adapter sequences, forward and reverse primer pair, target-specific primer, degenerate primer, and sequences configured to bind to a DNA sequencing platform substrate, and any other primer configuration recognized by one having skill in the art as effective for various amplification, single strand extension, or other related reactions. Primers can comprise various functional sequences useful for various reactions, including library preparation and amplification. Non-limiting examples of sequences that can be included in a primer are: a barcode (e.g., sample barcode), unique molecular identifier, non-hybridizable 5′ tail, flow cell sequence, sequence complementary to a flow cell sequence, and target-specific sequence. Primers can be greater than 15 bases in length. Primers can be about 18 to about 24 bases in length. In other instances primers can be about 28 to about 35 bases in length. Primers can be greater than 35 bases in length. Primers can be greater than 45 bases in length. One of skill in the art will recognize the appropriate primer length and composition to identify a nucleic acid sequence of interest.

Index sequences or tags can be used as nucleic acid sample preparation reagents. These oligonucleotide sequences can be single- or double-stranded and be used to identify a sample or fragment molecule. Index sequences or tags can also include sequence barcodes. An index can be used to differentiate fragments when sequencing many samples at once, as in multiplex sequencing reactions. Other oligonucleotide sequences can be used to identify nucleic acid molecules or samples. Oligonucleotide sequences such as these can each contain randomized, unique sequences or known, non-unique sequences. Oligonucleotide sequences can be greater than 5 bases or base pairs long. In some instances, these identification sequences can be about 8 to about 12 bases or base pairs long. In other instance they can be greater than 12 bases or base pairs long. In still other instances, the sequence identifiers can be 6 bases or base pairs long.

Other nucleic acid sample preparation reagents include adapter sequences. Adapter sequences can be double stranded, partially double stranded or single stranded. Adapter sequences can comprise various sequences useful for various reactions, sequencing platforms, or fragment identification. Examples of these sequences include: target specific sequences, universal sequences, index sequences, unique molecular identifier, sequences configured to bind to a flow cell, sequences configured to bind to a DNA sequencing platform substrate, sequences configured for paired end sequencing. In some instances, adapters can be configured for ligation with blunt or end-repaired DNA fragments. Adapters can be partially double stranded and comprise a complementary region and a non-complementary region. Adapters can be double stranded and comprise complementary sequences. Adapters can be adenylated and comprise a blocked 3′ end. Adapters can be configured for RNA library preparation. Adapters can be configured for ligation with blunt-end repaired DNA having a 3′ adenine base overhang. Adapters can be various lengths. Adapters can be greater than 30 bases or base pairs long. Adapters can be about 50 to about 70 bases or base pairs long. Adapters can be 60 bases or base pairs long. Adapters can be configured for various applications. In some instances, an adapter can comprise a region configured to bind to a flow cell or sequencing platform substrate. Adapter sequences can comprise an index sequence. Adapters can be configured for universal ligation to nucleic acid fragments. Adapters can additionally be configured for paired-end sequencing.

Nucleic acid sample preparation reagents can comprise nucleic acid probes, which can be stabilized, e.g., selectively stabilized, on the solid support matrix. Nucleic acid probes can be configured to hybridize to RNA or DNA. Nucleic acid probes can be about 10 bases or base pairs to about 10,000 bases or base pairs in length. Nucleic acid probes can be about 100 to about 1000 bases or base pairs in length. Nucleic acid probes can be fluorescent or radiolabeled. A stabilized nucleic acid probe can be configured for fluorescence in situ hybridization. In some instances, probes can be radiolabeled with radioisotopes including but not limited to phosphorus-32 (³²P), tritium (hydrogen-3) (³H), carbon-14 (¹⁴C), and iodine-125 (¹²⁵I). One of skill in the art will recognize how to configure nucleic acid probes to hybridize a nucleic acid sequence.

Nucleic acid sample preparation reagents can comprise various nucleotides. Nucleic acid sample preparation reagents can comprise nucleotide monophosphates, including adenine monophosphate, guanine monophosphate, cytosine monophosphate, thymine monophosphate, and uracil monophosphate. Nucleic acid sample preparation reagents can comprise nucleotide di-phosphates, including adenine di-phosphate, guanine di-phosphate, cytosine di-phosphate, thymine di-phosphate, and uracil di-phosphate. In some instances, dNTPs can have reversible terminator groups comprising a cleavable linker and a fluorescent dye. Nucleic acid sample preparation reagent can comprise deoxynucleoside triphosphates (dNTPs), including deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP). Nucleotide tri-phosphates can include dideoxynucleotide tri-phosphates (ddNTPs), including ddATP, ddCTP, ddGTP, ddUTP, and ddTTP; and ribonucleotide tri-phosphates including ATP, CTP, GTP, TTP, and UTP. Nucleic acid sample preparation reactions can also comprise non-canonical nucleotides including for example expanded bases containing an additional benzene ring to widen the DNA helix, Hirao bases, peptide nucleic acid, locked nucleic acid, glycol nucleic acid, and threose nucleic acid. Nucleic acid sample preparation reactions can also comprise nucleosides, e.g. ribonucleosides and deoxynucleosides, including adenosine, deoxy adenosine, guanosine, deoxyguanosine. 5-methyluridine, thymidine, uridine, deoxyuridine, cytidine, and deoxycytidine.

Sample preparation reagents can comprise protein. Protein sample preparation reagents can be configured for various reactions upon rehydration, including: sequencing reaction, extension reaction, amplification reaction, hybridization reaction, immunohistochemistry reaction. Non-limiting examples of protein sample preparation reagents include enzymes, antibodies, and fluorescent probes.

A solid support matrix can comprise enzyme sample preparation reagents therein. The enzyme sample preparation reagents can be stabilized, e.g., selectively stabilized, in the solid support matrix. Enzyme sample preparation reagents can comprise ligase (e.g. DNA ligase, RNA ligase), transposase, reverse transcriptase, nuclease (e.g. deoxyribonuclease, ribonuclease, exonuclease, endonuclease), polymerase (e.g., DNA polymerase, DNA-dependent RNA polymerase), terminal transferase, glycosylase, DNA fragmenting enzyme, ribonuclease, and polynucleotide kinase, phosphatase, pyrophosphatase, methylase, topoisomerase, guanylyl transferase, protease, telomerase, and any other enzymes known to be used in connection with the reactions listed above.

The solid support matrix can comprise a polymerase; the polymerase can be stabilized, e.g., selectively stabilized, within the solid matrix support. A polymerase can catalyze various reactions using DNA or RNA substrates. A stabilized, e.g., selectively stabilized, polymerase can be any one of the following and any other polymerase recognized by one skilled in the art as effective for nucleic acid amplification, replication, elongation, proof-reading and/or repair. Stabilized polymerases can have varying activities and can be configured for a variety of purposes. For example, some polymerases can be characterized by the following: polymerase with 3′ to 5′ exonuclease activity, polymerase that lacks 3′ to 5′ exonuclease activity, polymerase with 5′ to 3′ exonuclease activity, polymerase configured to generate blunt-ended nucleic acid products, and polymerase configured to have fidelity at least 3 fold greater than Taq DNA polymerase. Non-limiting examples of polymerase enzymes that can be stabilized, e.g., selectively stabilized, include: phi29 polymerase, T4 DNA polymerase, T7 DNA polymerase, Klenow DNA polymerase, Klenow fragment (3′ to 5′ exonuclease⁻), Bst DNA polymerase (full length or large fragment), Bsu DNA polymerase, E. coli DNA polymerase I, Top DNA polymerase, Taq DNA polymerase, and Pfu DNA polymerase, DNA polymerases configured to have 3′ to 5′ exonuclease activity, Sulfolobus DNA polymerase IV, high fidelity DNA polymerases configured to have fidelity at least 3 fold greater than Taq DNA polymerase, T3 RNA polymerase, T7 RNA polymerase, SP6 RNA polymerase, E. coli poly(A) polymerase, poly(U) polymerase, E. coli RNA polymerase core enzyme, E. coli RNA polymerase holoenzyme, T5 DNA exonuclease, or Taq Polymerase. One of skill of the art will recognize additional polymerase enzymes that can be selectively stabilized.

A solid support matrix can comprise a ligase enzyme; the ligase enzyme can be stabilized, e.g., selectively stabilized, within the solid support matrix. The ligase can be any one of the following: T4 DNA ligase, T7 DNA ligase, Taq DNA ligase, T4 RNA ligase 2, T4 RNA ligase 2 trunc, T4 RNA ligase 1, circligase, tRNA ligase, thermostable ATP-dependent ligase, and archaeal stable RNA ligase. One skilled in the art will recognize additional ligase enzymes that are effective for ligation reactions.

The solid support matrix can comprise a reverse transcriptase; the reverse transcriptase can be stabilized, e.g., selectively stabilized, within the solid support matrix. Reverse transcriptase can catalyze various reactions, including for example single strand elongation. Rehydrated reverse transcriptase can be used to catalyze first strand cDNA synthesis using a RNA substrate. Reverse transcriptase enzyme can be selected from the following: avian myeloblastosis virus (AMV) reverse transcriptase, moloney murine leukemia virus (M-MuLV, MMLV) reverse transcriptase, reverse transcriptase configured to initiate DNA synthesis from a primer using either a RNA or a DNA template, reverse transcriptase configured to have increased thermostability, reverse transcriptase configured for reduced RNaseH activity. One skilled in the art will recognize that other reverse transcriptase enzymes can be used in single-strand elongation reactions.

The solid support matrix can comprise a DNA repair enzyme; the DNA repair enzyme can be stabilized, e.g., selectively stabilized within the solid support matrix. DNA repair enzymes can comprise glycosylase or exonuclease. Several glycosylases can be stabilized including for example uracil glycosylase. Various exonucleases can be selectively stabilized. Stabilized exonucleases can comprise 3′ to 5′ or a 5′ to 3′ activity.

The solid support matrix can comprise an endonuclease enzyme; the endonuclease enzyme can be stabilized, e.g., selectively stabilized within the solid support matrix. A stabilized endonuclease enzyme can cleave a single strand of DNA or cleave double stranded DNA. Endonuclease enzymes can comprise a restriction endonuclease or an apurinic/apyrimidinic (AP) endonuclease.

The solid support matrix can comprise additional enzymes used in DNA sequencing reactions; the additional enzymes can be stabilized, e.g., selectively stabilized within the solid support matrix. These enzymes can include polynucleotide kinase, exonuclease, DNA fragmenting enzymes, RNaseH, DNase I, or other enzymes recognized by one skilled in the art as DNA sequencing sample preparation reagents. Stabilized enzymes can further include restriction endonuclease enzymes. Solid support matrix-stabilized restriction enzymes can be used in DNA fragmenting and recombinant DNA reactions, such as for example restriction endonuclease enzymes.

Other stabilized protein sample preparation reagents can comprise antibodies. Antibodies can be used in various immunohistochemistry reactions. As a non-limiting example, stabilized antibodies can be used to detect a target protein in a biological sample. In some embodiments, antibodies can be visually detected by labeling the antibody with an enzyme, a fluorescent molecule, radioactive isotypes, or other appropriate means for antibody labeling. Multiple distinct antibodies can be used in the detection of a protein. For example, a second fluorescent antibody can bind to a complex of a first antibody and a target protein. Enzymes, used alone or bound to an antibody, can also be selectively stabilized within the solid matrix to detect a stabilized protein. Chromogenic substrates can also be incorporated into and selectively stabilized within a solid support matrix. In one or more examples, if there is an enzyme (in a biological sample, for example) that catalyzes a chromogenic substrate (impregnated into the stabilization matrix, for example), the enzyme can react with the substrate so that a color change will occur and the enzyme can be quantified based on relative color change. One of skill in the art will recognize that it can be possible to selectively stabilize other reagents within the solid matrix to detect proteins. The examples provided above are exemplary and are not intended to be limiting.

The term “antibody” as used herein can refer to immunoglobulin molecules and immunologically active portions of immunoglobulin molecules, i.e., molecules that contain an antigen binding site that immunospecifically binds an antigen. The term can also refer to antibodies comprising two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH₂, F(ab′)2, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies).

The solid support matrix can comprise various combinations of reagents. The solid support matrix can include a combination of nucleic acid and protein sample preparation reagents. The solid support matrix can further include reagents that are not sample preparation reagents. For example, reagents can comprise any combination of reagents including a protein denaturant, a reducing agent, buffer, a free-radical trap, a chaotropic agent, a detergent, or an RNase inhibitor in the solid matrix in a dried format or combinations thereof. Non-limiting examples of suitable reagents for storage media can include one or more of a weak base, a chelating agent and optionally, uric acid or a urate salt or simply the addition of a chaotropic salt, alone or in combination with a surfactant. Examples of chaotropic salt include, but are not limited to, guanidine thiocyanate, guanidine chloride, guanidine hydrochloride, guanidine isothiocyanate, sodium thiocyanate, and sodium iodide. RNase inhibitors can comprise a triphosphate salt, pyrophosphate salt an acid, or an acid-titrated buffer reagent. The solid support matrix can further be impregnated with or in the presence of one or more reagents including enzyme inhibitors, free-radical scavengers, or chelating agents. The solid support matrix can comprise a protein denaturant, a reducing agent, a buffer, and optionally a free-radical trap or RNase inhibitor.

Various reactions can be performed by rehydrating a matrix-stabilized sample preparation reagent.

A reaction can be performed by rehydrating a solid support matrix comprising a sample preparation reagent within a container. A reaction can be performed by adding liquid to a solid support matrix comprising a sample preparation reagent, thereby rehydrating the sample preparation reagent. A liquid can comprise biomolecules (e.g., a nucleic acid or a protein). In some cases, the liquid does not comprise biomolecules. A liquid for rehydrating the solid support matrix can initially lack biomolecules and biomolecules can be later added. Sample preparation reagents can leave, e.g. be eluted from, a solid support matrix when rehydrated. Sample preparation reagents that leave a solid support matrix can perform a reaction in a solution. Reaction products can be in solution. Sample preparation reagents can stay within the solid support matrix. Sample preparation reagents can be tethered to the solid support matrix. Reaction products can stay within the solid support matrix. A solid support matrix can be placed in an instrument, e.g. a thermocycler, to perform a reaction. Supernatant from a rehydrated solid support matrix can be placed in an instrument, e.g. a thermocycler, sequencing platform, e.g., next-generation sequencing platform, e.g., nanopore sequencing platform, ion semiconductor quenching platform, or sequencing platform making use of bridge amplification and reversible dye terminators, to perform a reaction, without the solid support matrix.

A reaction, e.g., a sequencing reaction, can be performed by providing a solid support matrix for selectively stabilizing nucleic or proteins having a sample preparation reagent stabilized therein and rehydrating the sample preparation reagent. Rehydration can be achieved by applying a sample, water or any other solution (e.g. a buffer solution).

A reaction can involve nucleic acid library preparation. Nucleic acid libraries can be prepared for DNA or RNA. Exemplary reactions involved in nucleic acid library preparation include purification, fragmentation, end repair, removal of damaged bases, adenylation (e.g., 5′ or 3′ adenylation), adapter ligation, purification of ligation products, primer annealing, primer extension, amplification, and enrichment. Exemplary reactions using RNA, include first strand synthesis (e.g., reverse transcription), second strand synthesis, purifying cDNA templates, adaptor ligation, and enriching purified cDNA templates, e.g., by amplification. Nucleic acid libraries can be useful for various downstream applications including sequencing (e.g., next-generation sequencing, e.g., nanopore sequencing, sequencing using reversible dye terminators and bridge amplification, ion semiconductor sequencing), microarray, polymerase chain reaction e.g., quantitative PCR (qPCR), digital PCR, e.g., droplet digital PCR, mass spectrometry, and others recognized by one having skill in the art.

A sequencing reaction can be performed by rehydrating nucleic acid sample preparation reagents stabilized within a solid support matrix. The nucleic acid sample preparation reagents can include but are not limited to: primers, universal primers, random primers, oligodT primers, primers comprising a barcode, oligonucleotide sequences configured to index a nucleic acid sequence, single stranded adapter sequences, double stranded adapter sequences, oligonucleotide sequences configured to bind to a flow cell, oligonucleotide sequences configured to bind to a DNA sequencing platform substrate, oligonucleotide sequences comprising an adapter sequence and a flow cell binding site, adapter sequences configured for paired end sequencing, deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP). One skilled in the art will recognize that there will be additional nucleic acid sample preparation reagents not listed above which can be used in sequencing reactions.

Selectively stabilized nucleic acid sample preparation reagents can be utilized in various reactions. As a non-limiting example, primers and dNTPs can be selectively stabilized within a solid support matrix configured for nucleic acid stabilization. These reagents can be rehydrated for use in amplification or single strand elongation reactions. In a further example, a rehydrating solution can comprise enzymes such as DNA polymerase. Additionally, probes can be stabilized in the solid support matrix such that rehydration of the solid support matrix with a solution of nucleic acids can result in hybridization of the nucleic acid probe with the nucleic acids in the rehydrating solution.

A reaction can be performed by rehydrating protein reagents stabilized within a solid support matrix configured to stabilize proteins. Non-limiting examples of protein sample preparation reagents include: T4 RNA ligase 2, T4 RNA ligase 2 trunc, and T4 RNA ligase 1, T4 DNA ligase, T4 polynucleotide kinase, transposase, reverse transcriptase, exonuclease, DNA Polymerase I, Phi29 polymerase, T4 DNA polymerase, Klenow DNA Polymerase, Klenow Fragment (3′ to 5′ exonuclease⁻), Top DNA polymerase, Taq DNA polymerase, and Pfu DNA polymerase, DNA fragmenting enzyme, antibody, enzyme-labeled antibodies, fluorescent molecule labeled antibodies, radioactive antibody isotypes. One skilled in the art will recognize that there will be additional protein sample preparation reagents not listed above which can be used in sequencing reactions.

Stabilized protein reagents can be rehydrated to perform several types of reactions. Some of these reactions can be enzymatic, as is the case for single strand extension, amplification, and ligation reactions. For instance, selectively stabilized reverse transcriptase, RNaseH and DNA polymerase can be rehydrated to initiate cDNA synthesis. In another example, selectively stabilized ligase can initiate adapter ligation. In still another exemplary embodiment, stabilized protein sample preparation reagents including T4 DNA polymerase, Klenow DNA polymerase, and polynucleotide kinase can initiate DNA end repair, where the ends of fragmented DNA are blunted and the 5′ ends are phosphorylated using a polynucleotide kinase. In another example, stabilized Klenow Fragment (3′→5′ exonuclease−) can be rehydrated to initiate the adenylation of the 3′ end of a DNA fragment. Stabilized DNA fragmenting enzymes can initiate the fragmentation of DNA into 50 to 1000 base pair fragments.

Some reactions can be affinity reactions, such as for example immunohistochemistry reactions. Sample preparation reagents such as antibodies can be selectively stabilized to target protein(s) or nucleic acid sequence(s) for purification and later extraction. For instance, protein bio-components can bind to selectively stabilized antibodies within a solid support matrix configured to selectively stabilize protein. The antibodies can bind epitopes on particular protein(s) of interest. Later at a laboratory, bio-components can then be selectively eluted from the solid matrix-stabilized bio-component protein complexes. It can be desirable to first wash away non-bound sample components attached to the solid matrix using a buffer that maintains the antibody-protein binding interaction(s).

FIG. 8 generally describes an example of how a solid support matrix having sample preparation reagents stabilized therein can be used. A dry solid matrix having sample preparation reagents stabilized therein can be deployed to a user (110). The user can then rehydrate the dry solid support matrix (120), thereby initiating a reaction using at least the reagents stabilized therein (130). After a reaction is carried out to a desired degree, reaction product can then be eluted from the matrix (140).

A solid support matrix that selectively stabilizes nucleic acids or proteins and a sample preparation reagent stabilized therein can be included in a kit. In some instances, the kit can be used to perform various reactions, including sequencing reaction, extension reaction, amplification reaction, hybridization reaction, immunohistochemistry reaction, ligation reaction, end repair reaction, adenylation reaction. For example, dry, matrix stabilized sample preparation reagents can be rehydrated and used directly in polymerase chain reaction. A kit can comprise one solid support matrix with sample preparation reagents stabilized therein. A kit can comprise two or more solid support matrices with different sample preparation reagents stabilized therein. To provide a non-limiting example, a kit can comprise a solid support matrix configured to selectively stabilize protein and a solid support matrix configured to selectively stabilize nucleic acids. This kit can be configured to perform amplification (such as for example in the case of polynucleotide synthesis). In such an instance, the solid support matrix configured to selectively stabilize protein can have DNA polymerase stabilized thereon. The solid support matrix configured to stabilize nucleic acids can have nucleic acid sample preparation reagents such as dNTPs and primers stabilized therein. The quantities of sample preparation reagent stabilized therein can be appropriate for a given quantity of sample by weight, sequence length, or moles. The amount of matrix included in the kit can be based in part on a quantity of sample preparation reagent per square unit of solid support. Upon rehydration, the solid support matrices can be used for amplification in applications such as polymerase chain reaction. As a non-limiting example, the two-matrix kit can be contained in a single vessel, rehydrated, thereby initiating amplification.

In some embodiments, the solid matrix can selectively stabilize blood plasma components. Plasma components can include cell-free DNA, cell-free RNA, protein, hormones, and other metabolites, which can be selectively stabilized on the solid matrix. Plasma components can be isolated from whole blood and stabilized on a solid matrix. A solid matrix can be overlapping with or a component of a variety of different devices and techniques. Plasma components can be separated from whole blood samples using a variety of different devices and techniques. Techniques can include lateral flow assays, vertical flow assays, and centrifugation.

A solid matrix can be integrated with or a component of a variety of plasma separation devices or techniques. A solid matrix can be overlapping with or a component of a variety of different devices, such as a plasma separation membrane for example Vivid™ plasma separation membrane. A solid matrix can partially overlap with plasma separation device such as a plasma separation membrane. Examples of devices and techniques for plasma separation are disclosed in patents or patent publications, herein incorporated by reference, including U.S. Pat. Nos. 6,045,899; 5,906,742; 6,565,782; 7,125,493; 6,939,468; EP 0,946,354; EP 0,846,024; U.S. Pat. Nos. 6,440,306; 6,110,369; 5,979,670; 5,846,422; 6,277,281; EP 1,118,377; EP 0,696,935; EP 1,089,077, US 20130210078, and US 20150031035.

In various devices and techniques, a separation membrane can be used. The separation membrane can be comprised of polycarbonate, glass fiber, or others recognized by one having skill in the art. Membranes can comprise a solid matrix. Membranes can have variable pore sizes. Separation membranes can have pore diameters of about 1 μm, about 2 μm, about 4 μm, about 6 μm, about 8 μm, about 10 μm, about 12 μm, about 14 μm, about 16 μm, about 18 μm, about 20 μm. A separation membrane can have pores with diameters of about 2 μm to about 4 μm. A separation membrane can have pores that are about 2 μm in diameter.

Plasma separation can be implemented for a wide variety of sample volumes. Plasma sample volumes can be variable depending on the application for which a solid matrix is used. Sample volumes can be greater than about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL. Sample volumes can range from about 250 μL to about 500 μL.

Other examples of stabilization matrix or stabilization components that can be used in the devices and methods described include, but are not limited to Gentegra-RNA, Gentegra-DNA (Gentegra, Pleasanton CA), as further illustrated in U.S. Pat. No. 8,951,719; DNA Stable Plus, as further illustrated in U.S. Pat. No. 8,519,125; RNAgard Blood System (Biomatria, San Diego, CA).

### Preparation of Bio-Components

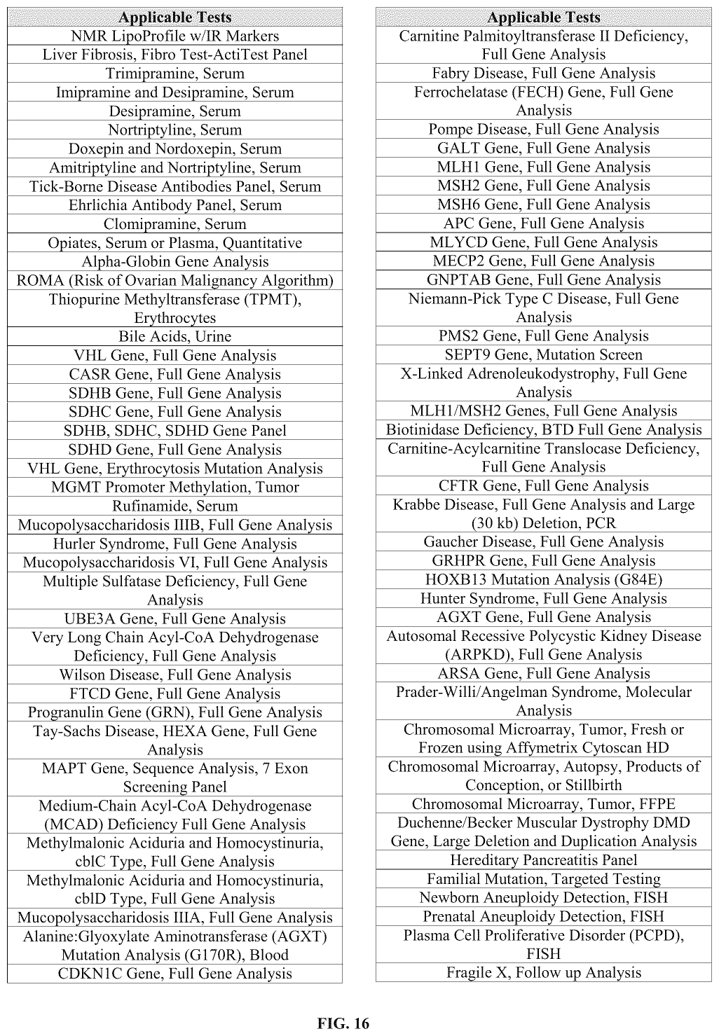

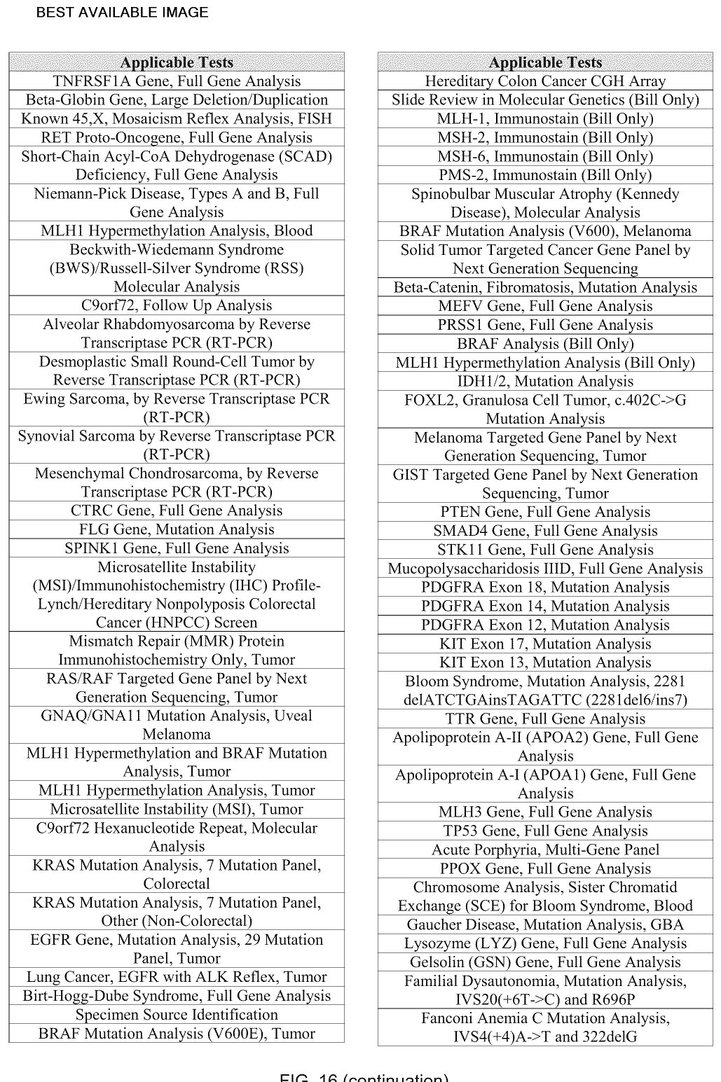

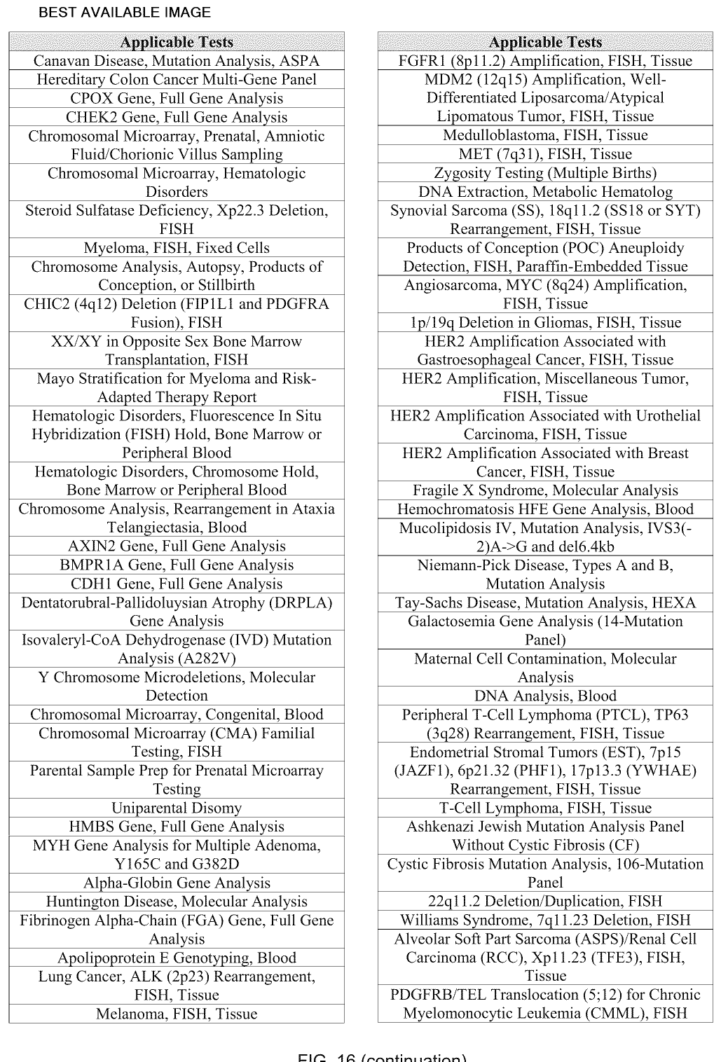

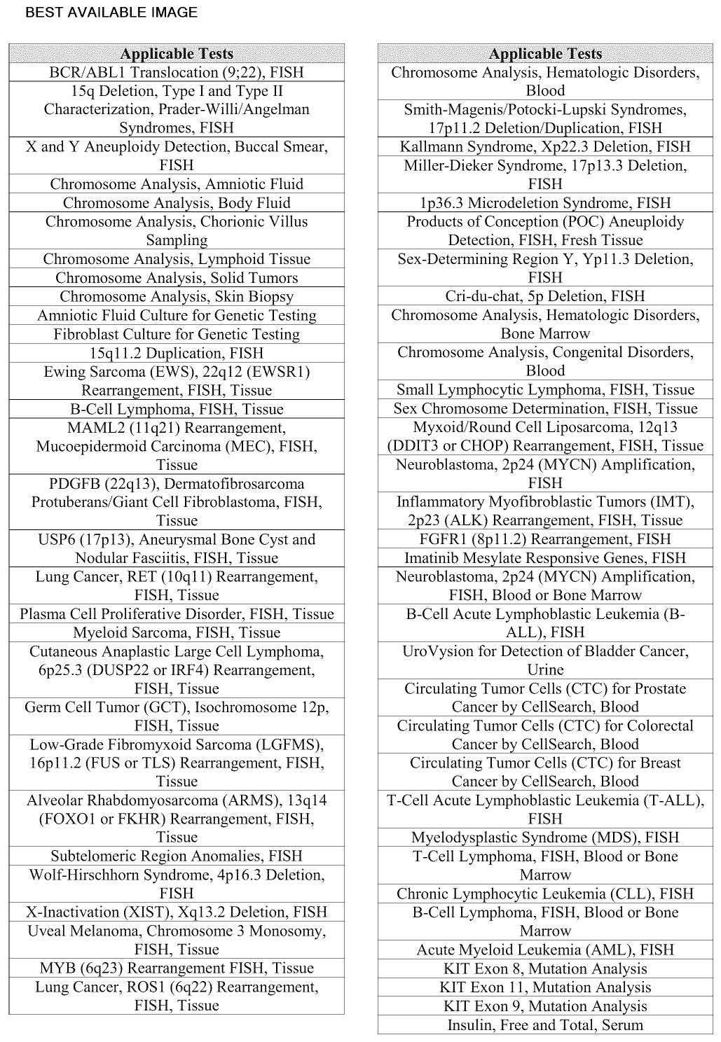

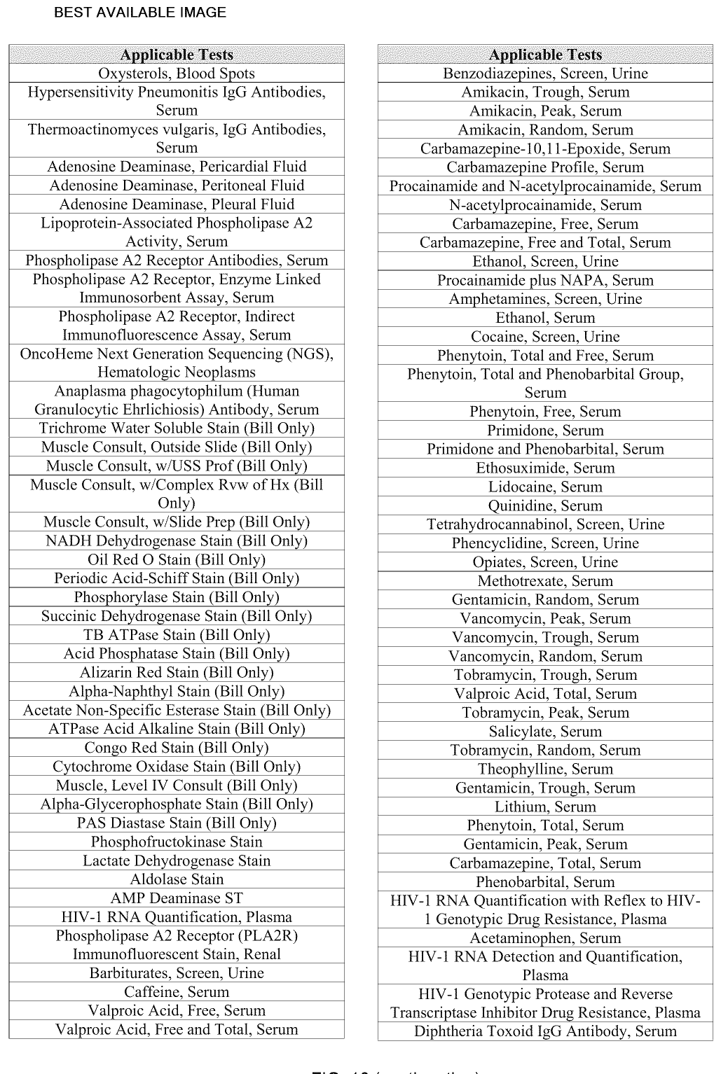

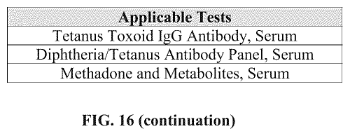

In some embodiments the user or operator can remove components of the system before sending the sample off to a facility for analysis. The facility can be a CLIA facility, a laboratory, medical office or external dedicated facility. At facility, the samples can be used in any diagnostic tests including but not limited to common panels, thyroid tests, cancer diagnostic tests, tests and screens for cardiovascular disease, genetic diseases/pre-natal testing and infectious disease. A non-limiting list of applicable tests is herein included as FIG. 16. In some embodiments tests can detect one or more of the following: alpha-fetoprotein (AFP), pregnancy associated plasma protein A (PAPP-A), human chorionic gonadotropin (hCG), unconjugated estriol (uE3), and dimeric inhibin A (DIA).

In some embodiments, more than one test can be administered. In further embodiments, tests can be administered over defined time increments. For example, a baseline test can be administered and follow-up tests can be administered in pre-defined increments. Pre-defined increments can be regular, for example every month, or irregular. Irregular increments can be defined based on the outcome of the tests. Increments can include duration(s) of: 1 day, 3 days, 5 days, 1 weeks, 2 weeks, 3 weeks, 1 month, 2 months, 3 months, 4 months, 5 months, 6 months, 1 year, 2 years, or 5 years.

The devices and systems for acquiring, collecting, separating and stabilizing samples can be modular; comprising distinct compartments or components. In some cases, the distinct components are or are not easily removed or separated. Separable or removable components can include the sample substrate, or a sample separation component.

The devices herein (e.g., sample acquisition components and sample stabilization component) can be transported together to a laboratory for further analysis of the bio-components. Alternatively, the stabilization component (e.g., substrate or substrate) can be shipped without the sample acquisition components to the laboratory for further analysis of the bio-components. In some instances, treatment and analysis can be performed on the deployed device, systems or substrate, and either the stabilization component or a component of the device or system can be transported to a healthcare provider or other party interested in the results of the test. Once a bio-sample is received at a location for analysis, the substrate, or components of the sample separation component can be removed. These components can be tagged and/or labeled to indicate the composition or target component that is stabilized on the matrix. Using the tag as an identifier the membranes or substrates can be sorted and prepared for the target test.

Systems and processes for receiving and processing the samples can vary depending on the identity, stability, source or other features of the deployed samples. The quality of a bio-sample component can directly impact the quality, reproducibility and reliability of a diagnostic test result. The aforementioned systems, devices, and methods for sample acquisition, collection, stabilization, and optional separation, can impact the sample composition, stability, concentration, and processing. For example the volume, size, quantity, stability and purity of a bio-sample can vary depending on the sample source and the components used to collect the sample. The quality of the samples and by extension the quality of the diagnostic results can be specific the kits, systems, devices and methods for acquiring the sample; therefore, methods, kits, and systems are also disclosed for receiving, processing, treating and/or preparing components of the bio-sample prior to analysis.

Components processed from the sample can include but are not limited to DNA/RNA including cell-free nucleic acids, cell-free fetal DNA, cell-free maternal DNA and circulating-tumor DNA, proteins, antigens, antibodies, lipids including HDL/LDL, and any combination thereof. The bio-sample or target components, can be extracted using any of the conventional nucleic acid extraction methods. Non-limiting examples of extraction methods that can include but are not limited to, electroelution, gelatin extraction, silica or glass bead extraction, guanidine-thiocyanate-phenol solution extraction, guanidinium thiocyanate acid-based extraction, centrifugation through sodium iodide or similar gradient, centrifugation with buffer, or phenol-chloroform-based extraction. For nucleic acid analysis the extraction step can help remove impurities such as proteins and concentrates the circulating nucleic acids. Extracted circulating nucleic acids can be inspected using methods such as agarose gel electrophoresis, spectrophotometry, fluorometry, or liquid chromatography.

DNA or nucleotides extracted from the sample can be prepared at a lab facility using various methods for reducing error and producing significant signal with limited sample size. Nucleic acid samples can be treated or subjected to various methods for efficient amplification of the desired target nucleic acid (e.g., “DNA template” or “nucleic acid template”). Modified primers can be designed to minimize or prevent the production of unwanted primer-dimers and chimeric products observed with other nucleic acid amplification methods and kits. Primer design methods can avoid the production of spurious nucleic acid amplification products. The methods and kits described used for analyzing the sample can comprise “AT GenomiPhi.” ATGenomiPhi can use modified hexamers are of the general formula: +N+N(atN)(atN)(atN)*N, wherein “+” precedes an LNA base, as described above, and (atN) represents a random mixture of 2-amino-dA, dC, dG, and 2-thio-dT. Other hexamers can comprise the formula (atN)(atN)(atN)(atN)(atN)*N, wherein the notations are consistent between these two hexamer designs. The use of these hexamers in nucleic acid amplification techniques can address, minimize or eliminate the problems associated with the production of primer-dimer formation and chimeric nucleic acids observed in traditional methods by inhibiting the ability of the random hexamers to anneal with one another, by increasing the melting Tₘ of the primers, improving the binding efficiency of the hexamer to the target nucleic acid via the addition of LNAs and 2-amino-dA to the primers, and preventing annealing of the target DNA to itself through the incorporation of 2-thio-dT into the random hexamers. Moreover, the primer modifications can increase their binding strength to the target nucleic acid and permit the utilization of more stringent hybridization buffers that further minimize the likelihood of the production of primer-dimers and chimeric nucleic acid products. These and other methods of DNA amplification methods can be found in US Appn. No. US20130210078.

DNA derived from the sample can be analyzed using methods for generating single-stranded DNA circles from a biological sample. The laboratory analyzing the sample can use a method comprising the steps of: treating the biological sample with an extractant to release nucleic acids, thereby forming a sample mixture; neutralizing the extractant; denaturing the released nucleic acids to generate single-stranded nucleic acids; and contacting the single-stranded nucleic acids with a ligase that is capable of template-independent, intramolecular ligation of a single-stranded DNA sequence to generate single-stranded DNA circles. The steps of the method can be performed without any intermediate nucleic acid isolation or nucleic acid purification. In certain embodiments, the steps can be performed in a sequential manner in a single reaction vessel. In certain embodiments, the single-stranded DNA circles can be amplified to enable subsequent analysis of the biological sample. In certain embodiments, the sample mixture can be dried on solid matrix prior to the neutralizing step. In certain embodiments, damage to the DNA can be repaired enzymatically prior to the denaturing step. In other aspects, the method is provided for analyzing a biological sample. Thus, the single-stranded DNA circles generated according to certain embodiments of the method are amplified, and the amplification product is analyzed. The analysis can be performed by, for example, targeted sequencing of the amplified product. In another aspect of the invention, a method is provided for detecting chromosomal rearrangement breakpoints from a biological sample. Thus, the single-stranded DNA circles generated according to certain embodiments of the invention are amplified, and the amplification product is analyzed, e.g., by sequencing. Any chromosomal rearrangement breakpoints can be identified by comparing the sequences to a known reference sequence. In yet another aspect of the invention, a kit can be provided that comprises an extractant for treating a biological sample to release nucleic acids; a reagent for neutralizing the extractant; and a ligase that is capable of template-independent, intramolecular ligation of a single-stranded DNA sequence. These and other method of DNA amplification methods can be found in PCT Appn. No. WO US2015/50760.

Methods for generating a single-stranded DNA circle from a linear DNA can be used on the collected sample. The methods can comprise steps for providing a linear DNA, end-repairing the linear DNA by incubating it with a polynucleotide kinase in the presence of a phosphate donor to generate a ligatable DNA sequence having a phosphate group at a 5′ terminal end and a hydroxyl group at a 3′ terminal end, and performing an intra-molecular ligation of the repaired, ligatable DNA sequence with a ligase in order to generate the single-stranded DNA circle. All steps of the method can be performed in a single reaction vessel without any intervening isolation or purification steps. The phosphate donor can be a guanosine triphosphate (GTP), a cytidine triphosphate (CTP), a uridine triphosphate (UTP), a deoxythymidine triphosphate (dTTP) or a combination thereof. The linear DNA can either be double-stranded or single-stranded DNA. DNA can be a segment of fragmented DNA such as circulating DNA. The ligatable DNA, if in double-stranded form, can need to be denatured prior to intra-molecular ligation reaction. A pre-adenylated ligase that is capable of template-independent, intra-molecular ligation of single-stranded DNA sequences can be employed for the ligation reaction. In other embodiments, the method for generating a single-stranded DNA circle from a linear DNA can employ a DNA pre-adenylation step prior to an intra-molecular ligation step. The linear DNA can optionally be incubated with a polynucleotide kinase in the presence of adenosine triphosphate (ATP) to generate a ligatable DNA sequence that comprises a phosphate group at a 5′ terminal end and a hydroxyl group at a 3′ terminal end. Generation of a ligatable DNA sequence from the linear DNA can be preferred if the linear DNA is in a highly fragmented form. The linear DNA or the ligatable DNA sequence can then be incubated with an adenylating enzyme in presence of ATP to generate a 5′ adenylated DNA sequence. The 5′ adenylated DNA sequence can be incubated with a non-adenylated ligase, which is capable of template-independent intra-molecular ligation of the 5′ adenylated DNA sequence to generate the single-stranded DNA circle. All steps of the method can be performed in a single reaction vessel without any intervening isolation or purification steps. ATP can have to be removed from the reaction mixture (e.g., by treating the reaction mixture with a phosphatase) before the intra-molecular ligation reaction if the non-adenylated ligase is an ATP-dependent ligase. If the 5′ adenylated DNA is in double-stranded form, it can need to be denatured prior to the intra-molecular ligation reaction. These and other method of DNA circularization and amplification methods can be found in US Pub. No. US20150031086.

Sample treatment can involve steps to prepare RNA for analysis. The methods can involve the production of a nucleic acid structure and its subsequent use in the purification and amplification of nucleic acid. The methods can require a DNA sequence that comprises a double stranded region and a single stranded region. The single stranded region is complementary to the RNA sequence of interest. The RNA sequence is then hybridized to the single stranded region of the DNA sequence and then the two sequences are ligated in a novel procedure to produce an RNA-DNA molecule. Methods can include steps whereby the 3′ end of RNA is ligated to a double stranded DNA oligonucleotide containing a promoter sequence. This double stranded DNA oligonucleotide can contain a promoter for RNA polymerase within the double stranded region that is followed by a segment of single stranded DNA forming a 3′ overhang. When the 3′ overhang contains a string of thymidine residues, the single stranded portion of the double stranded DNA can hybridize to the 3′ end of messenger RNA (mRNA) poly(A) tails. After the addition of ligase mRNA can have one strand of double stranded DNA sequence ligated to the 3′ end. When an RNA polymerase is added, the RNA-DNA hybrid molecules can be efficiently transcribed to synthesize cRNA. As transcription reactions using RNA polymerase typically transcribe each template multiple times, this method can allow for effective RNA amplification. Another method similar to that described above can involve the ligation of the DNA oligonucleotide to the RNA as described. However, the DNA oligonucleotide can either attach to a solid matrix or contain an affinity tag. This can allow for very efficient covalent attachment and/or capture of RNA molecules, which can be used for any of a variety of purposes. Additional methods can utilize ligation and subsequent transcription to create complementary RNA containing a user-defined sequence at the 5′ end of the cRNA. This sequence “tag” can be placed between the RNA polymerase promoter and the 3′ end of the ligated RNA molecule. The user-defined sequence can be used for purification or identification or other sequence specific manipulations of cRNA. If cRNA product is subsequently ligated and re-amplified according to the described method, the resulting doubly-amplified product will be “sense”, with respect to the original sense template and this new product can have two separate user-defined sequences located at the 5′ ends. These sequences can be used for synthesis of cDNA, allowing for full-length synthesis and directional cloning. Those skilled in the art will understand that either with or without the user defined sequences this double amplification method can provide a significant increase in RNA quantity, allowing for analysis of samples previously too small for consideration. These and other methods for amplification of DNA fragments can be found in US Appn. No. US20080003602.

Additional methods for analysis of the bio-sample can focus on nucleic acids present in a region of interest in a biological sample, and provide protein expression information for many proteins as well as add to the DNA sequence information. Additional methods for sample analysis can focus on homogeneous subsection of a heterogeneous sample. One advantage is that specific subpopulations of cells within mixed populations can be accurately identified from predetermined selection criteria and analyzed. A further advantage is that mutations can be identified, which can be useful for diagnosis and/or prognosis or for further investigation of drug targets. These and other methods for DNA amplification can be found in PCT Appn. No. US2015/50760.

Nucleic acids can undergo any of the above mentioned sequencing reactions or methods. Further sequencing reactions or methods can include next-generation sequencing alone, in combination with any previously mentioned sequencing methods or reaction components, or with other methods of sequencing or analysis that are used in the field of nucleic acid sequence detection (e.g. RT-PCR, q-PCR etc.).

In some embodiments sample or selected components of the sample can be stabilized for future detection, screening, or diagnosis of a disease or disease state. In some embodiments, a sample or one or more components of a sample stabilized on a solid stabilization matrix can be use in a method to screen, detect, and/or diagnose a condition or disease state. Examples of tests that can be conducted on a sample are included in FIG. 16. Examples of conditions and disease states include fetal aneuploidy, pre-eclampsia, gestational diabetes and cancer.

### Applications of Stabilization Technology

Methods for using the systems and devices disclosed herein can include methods for analyzing one or more components of a sample. In some embodiments, sample components can comprise cell-free nucleic acids. The devices and systems disclosed herein can be used to collect, stabilize, and/or store the sample components. In some instances, cell-free nucleic acids can be obtained in a dehydrated state. For example, cell-free nucleic acids can be obtained on a solid matrix. In many instances the sample can have been exposed to the solid matrix in a liquid state, and the solid matrix can have one or more components, reagents, and/or structural modifications configured for selectively stabilizing one or more components of the sample on a solid matrix. Selective stabilization can include for example, use of components that bind to or sequester cell-free nucleic acids from other components for example proteins or enzymes. In some instances selective stabilization can involve destabilizing, destroying, or inhibiting non-selectively stabilized components of the sample. In other non-limiting instances, selective stabilization can include protecting the selectively stabilized component, for example with UV protectants or other measures or means for preserving the selectively stabilized component. In some cases, selective stabilization can include reagents or components that facilitate rapid drying of the sample or high levels of exposure of stabilizing components. Further examples of selective stabilization can include one or more systems, devices, components, or solid matrices for separating sample components prior to drying or storing the sample.

In some instances sample components can be separated during selective stabilization. In other instances, sample components can have been separated prior to sample stabilization. Methods for applying stabilization technology can include selective stabilization of nucleic acids, wherein the cell-free nucleic acids are from a cell-free sample from a subject. Cell-free samples from a subject can include any samples containing cell-free nucleic acids or other cell-free components that can be used for diagnosis or screening for bodily conditions. In some instances cell-free components can include cell-free proteins. Cell-free proteins can be circulating freely in fluid derived from the organism or released from a cell derived from the organism upon cell lysis. Cell lysis can occur on the solid matrix or prior to exposure to the solid matrix. Cell-free samples from a subject can comprise dried and/or selectively stabilized cell-free nucleic acids, cell-free proteins, or cell-free nucleic acids and cell-free proteins. The nucleic acids can be of viral, bacterial, fetal, mitochondrial, and genomic origins.

### Cell-Free Nucleic Acid Applications

In some instances, cell-free samples can be derived from a subject. The subject can have been, or can be diagnosed with a condition or suspected of having a condition. Examples of conditions include pregnancy and non-pregnancy related conditions. In some instances, a sample from a pregnant subject can include cell-free, and/or circulating nucleic acids. Some of the cell-free nucleic acids can include cell-free fetal DNA and cell-free maternal DNA. In some embodiments, the sample including cell-free components can be used to determine the presence or absence of a pregnancy-related or fetus-related condition.

In some instances the sample, which can include cell-free components, can be used to assess, screen for, or diagnose, a fetus-related condition or one or more characteristics of the fetus. Characteristics that can be assessed include: the gender of the fetus, epigenetic fetal markers, and or the paternity of the fetus. Fetus-related conditions can include X-linked conditions, fetal aneuploidy. A sample derived from the subject, with one or more selectively stabilized components, can be analyzed and used to assess fetal aneuploidy. In some instances, analysis or assessment can include steps for analyzing a monogenic disease. Monogenic diseases include but are not limited to the following: cystic fibrosis, beta-thalassemia, sickle cell anemia, spinal muscular atrophy, myotonic dystrophy, fragile-X syndrome, Duchenne muscular dystrophy, Hemophilia, achondroplasia, or Huntington's disease.

In some instances, a sample derived from a subject can be used to assess, diagnose, or screen for a pregnancy-related condition. In these instances, a pregnancy-related condition can include preeclampsia and gestational diabetes. Cell-free nucleic acids can also be used to perform a Rhesus or Rh factor test.

In some instances sample derived from a subject can be used to assess, screen, or diagnose a subject for one or more non-pregnancy related conditions. Examples of conditions that can be assessed, screened, or diagnosed include cancer, neurological diseases, neurodegenerative conditions, cardiac conditions, liver diseases, bacterial or viral infections, or autoimmune diseases. A condition that can be assessed, screened, or diagnosed is transplant graft injury or transplant rejection, e.g., heart transplant rejection. A sample derived from a subject can be used for early-stage screening, late-stage diagnosis, or on-going post-treatment monitoring of conditions, for example in the screening, diagnosis or post-treatment monitoring of a subject suspected of or treated for cancer. In some instances, a sample derived from a subject can comprise cell-free nucleic acids including, for example, cell-free nucleic acids from a tumor cell, for example dying tumor cells which can release circulating tumor DNA. Cell-free nucleic acids can also include cell-free DNA from non-tumor cells.

Cell-free nucleic acids can include cell-free DNA, cell-free RNA, and microRNA. Examples of cell-free nucleic acids include nucleic acids released from blood cell break down, break down from pathogens (e.g. bacteria or viruses), leucocyte surface DNA, DNA released as a result of cellular processes including apoptosis, necrosis, and pregnancy. Cell-free DNA can be formed due to spontaneous release of a newly synthesized DNA, or from spontaneous release of DNA/RNA-lipoprotein complex from healthy cells. Cell-free nucleic acids can comprise small fragments of 10 to 1000 base pairs. In some instances cell-free nucleic acids can be derived from plasma. In some instances cell-free nucleic acids can be associated with protein complexes, or exosomes.

In some instances circulating nucleic acids can comprise fetal and/or maternal cell-free nucleic acids. In these instances, cell-free fetal nucleic acids can increase over the course of pregnancy. In further embodiments, the cell-free nucleic acids can increase to greater than or equal to 10%, 15%, 20%, or 25% of the total cell-free nucleic acids in plasma derived from the subject. In some instances cell-free fetal nucleic acids can be rapidly cleared post-partum.

Cell-free nucleic acids can be released as a result of cellular processes including necrosis and apoptosis. In some instances necrosis can be caused by factors that are external to the cell. Examples of factors that can trigger necrosis and cell-free nucleic acid release include infections, toxins, and trauma. During necrosis the contents of a cell can be released and nucleic acids can degrade into nucleosomal units. In some instances apoptosis, or programmed cell death, can lead to release of cell-free nucleic acids. In these instances, cells can fragments into apoptotic bodies for digestion by phagocytes. In any of the aforementioned circumstances cell-free nucleic acids and any associated components can be collected, stabilized, detected, and or analyzed by the systems, devices, and methods disclosed herein.

A sample can be obtained in a liquid state. For example, a small volume liquid sample of less than 5 mL, 4 mL, 3 mL, 2 mL, 1 mL, or 0.5 mL can be used to assess, screen, or diagnose a pregnancy related or non-pregnancy related condition, using, e.g., cell-free nucleic acid or circulating nucleic acid from the sample from the subject. In some instances the small volume sample can be derived from a subject, and used to screen for one or more disorders. A small volume sample can comprise one or more cell-free components obtained or selectively stabilized in a liquid state, or stabilized on a matrix described herein. In some instances a small volume sample can be used to screen for the presence or absence of non-pregnancy or pregnancy-related conditions.

A small volume liquid sample can be obtained from a subject that is pregnant or suspected of being pregnant. In these instances, a small volume liquid sample can be used as part of a method of screening for a presence or absence of a fetal aneuploidy. A method can comprise obtaining cell-free fetal DNA and cell-free maternal DNA from a volume of less than 5 mL, 4 mL, 3 mL, 2 mL, 1 mL, or 0.5 mL of a liquid sample from a subject that is pregnant or suspected of being pregnant. The sample can be used to detect the presence or absence of a fetal aneuploidy using the cell-free fetal DNA and cell-free maternal DNA from the volume of less than 5 mL, 4 mL, 3 mL, 2 mL, 1 mL, or 0.5 mL of the liquid sample. Examples of fetal aneuploidy that can be assessed, screened or diagnosed, comprises trisomy 21, trisomy 18 and trisomy 13. A small volume liquid sample can comprise a sample of volume less than or equal to 5 ml, less than 4 mL, less than or equal to 3 mL, less than 2 mL, less than or equal to 1 mL, less than 500 μL less than or equal to 150 μL, less than 100 μL, less than 50 μL, less than 20 μL, less than 10 μL, or less than 5 μL.

In some embodiments, a small volume liquid sample can comprise cell-free fetal DNA and cell-free maternal DNA on a solid matrix before the sample is used to assess, screen, or detect for a condition. In some instances a method can comprise a step wherein cell-free fetal DNA and cell-free maternal DNA can be selectively stabilized on the solid matrix. In some instances the cell-free fetal DNA and cell-free maternal DNA can be dried on a solid matrix prior to assessment, screening, or detection of the sample. A method can comprise a step of rehydrating the dried cell-free fetal DNA and cell-free maternal DNA prior to the detecting. Screening, assessment or detection can include a detecting step that comprises sequencing. Sequencing can include next-generation sequencing, e.g., using a nanopore sequencing platform, ion semiconductor sequencing platform, or a platform that makes use of reversible dye terminators and bridge amplification.

A variety of liquid samples can be detected using the methods disclosed herein. Examples of samples can include whole blood, urine, and cerebrospinal fluid. In some instances, the liquid sample can be filtered prior to detection, assessment, screening, or analysis. In some instances the cell-free fetal DNA and cell-free maternal DNA are from plasma or serum, wherein the plasma or serum is derived from the whole blood. In some instances cell-free nucleic acids can be derived from blood, plasma, cerebrospinal fluid, and/or urine. Cell-free nucleic acids can be provided in a liquid sample, for example a liquid biopsy sample.

Cell-free nucleic acids from the sample can be collected and sequenced. Information can be derived from the presence, absence, or concentration of a particular cell-free nucleic acid sequence, or from the relative concentration of a particular sequence or fragment of cell-free nucleic acid. Sequencing can allow for detection and analysis. Analysis can comprise examination of concentration of particular fragments or markers. In some instances fragments or markers of cell-free nucleic acids can be examined as a concentration or a relative ratio, for example to determine of the percentage of one or more mutations or markers.

In some instances, cell-free nucleic acids can be detected using anon-invasive method for identifying, screening, or diagnosing a subject. The method can comprise a step of acquiring a baseline sample from a subject. In some instances the subject can be suspected of being pregnant or can be pregnant. The baseline sample comprises one or more cell-free nucleic acids. Additional steps in the method can include receiving one or more subsequent blood samples from the subject. The one or more subsequent samples comprise one or more cell-free nucleic acids. The method can further comprise an integrative analysis of the cell-free nucleic acids from the baseline sample with cell-free nucleic acids from the one or more subsequent samples; and estimation of the risk of the subject. In some instances, integrative analysis can comprise assessing parameters including the age of the patient, genetic predispositions, family history, and biometric data including blood pressure, weight, and other factors. In instances where the subject is pregnant or suspected of being pregnant, the integrative analysis can include pregnancy specific parameters including information from previous pregnancies.

In instances where the subject is pregnant or suspected of being pregnant, the baseline samples can be taken prior to or early in the pregnancy, for example in the first trimester. Subsequent samples can be derived from the subject during any individual or combination of the first trimester of pregnancy, the second trimester of pregnancy, or the third trimester of pregnancy.

Cell-free and/or circulating nucleic acids from a sample, e.g., less than 5 mL, 4 mL, 3 mL, 2 mL, 1 mL, or 0.5 mL of a sample (e.g., whole blood) from a subject that is pregnant or suspected of being pregnant can be applied to a matrix described herein, e.g., a matrix comprising sample preparation reagents, and the sample preparation reagents can be used to generate a nucleic acid library that can be used for sequencing, e.g., next-generation sequencing, and the generated sequence information can be used to detect a presence or absence of a fetal aneuploidy.

Cell-free and/or circulating nucleic acids from a sample e.g., less than 5 mL, 4 mL, 3 mL, 2 mL, 1 mL, or 0.5 mL of a sample (e.g., whole blood) from a subject that has cancer or is suspected of having cancer can be applied to a matrix described herein, e.g., a matrix comprising sample preparation reagents, and the sample preparation reagents can be used to generate a nucleic acid library that can be used for sequencing, e.g., next-generation sequencing, and the generated sequence information can be used to detect, diagnose, or monitor the cancer, or make a treatment (e.g., drug treatment) decision regarding the cancer.

### Protein-Based Applications

Methods for determining a presence or absence of a condition, or a likelihood of a condition, are also provided herein. Methods can comprise steps for selectively stabilizing protein from a sample from a subject on a solid matrix. In some instances a solid matrix for stabilizing a protein can be configured to reduce or minimize conformational changes or structural changes in a protein as a result of drying. Examples of reagents that can be adsorbed onto or absorbed into a solid matrix include melezitose. Additional steps can include analyzing the stabilized protein; and determining a presence or absence of a condition, or a likelihood of a condition, based on the analyzing. Cell-free or circulating proteins from a sample, e.g., less than 5 mL, 4 mL, 3 mL, 2 mL, 1 mL, or 0.5 mL of sample (e.g., whole blood), can be applied to a matrix provided herein and the protein can analyzed to detect or diagnose a fetal condition or a disease, e.g., cancer.

Conditions can include non-pregnancy and pregnancy related conditions. In instances where the presence or absence of a pregnancy-related condition is being determined, a sample can be obtained from a subject that is pregnant of suspected of being pregnant. Examples of pregnancy related conditions include fetal aneuploidy, gestational diabetes and preeclampsia. In some instances one or more proteins can be selectively stabilized. Examples of proteins that can be selectively stabilized and or assayed, detected, screened or analyzed include: alpha-fetoprotein (AFP), pregnancy associated plasma protein A (PAPP-A), human chorionic gonadotropin (hCG), unconjugated estriol (uE3), or dimeric inhibin A (DIA).

In some instances, sample comprising protein can be detected to identify, screen, monitor, or diagnose a condition in the subject. The method can comprise a step of acquiring a baseline sample from a subject. In some instances the subject can be suspected of being pregnant or can be pregnant. The baseline sample can comprise one or more dried samples. Additional steps in the method can include receiving one or more subsequent samples from the subject. The one or more subsequent samples comprise one or more dried samples with one or more selectively stabilized bio-components. The method can further comprise an integrative analysis of the proteins from the baseline sample with proteins from the one or more subsequent samples; and estimation of the risk of a condition in a subject. In some instances, integrative analysis can comprises assessing parameters including the age of the patient, genetic predispositions, family history, and biometric data including blood pressure, weight, and other factors. In instances where the subject is pregnant or suspected of being pregnant, the integrative analysis can include pregnancy specific parameters including information from previous pregnancies.

In instances where the subject is pregnant or suspected of being pregnant, the baseline samples can be taken prior to or early in the pregnancy, for example in the first trimester. Subsequent samples can be derived from the subject during any individual or combination of the first trimester of pregnancy, the second trimester of pregnancy, or the third trimester of pregnancy.

### Systems, Devices and Components

The herein presented systems, devices and methods can comprise multiple components including (i) a sample acquisition component (SAC) for simple collection of one or more bio-samples (e.g., blood, urine, or environmental samples such as water or soil), (ii) one or more stabilization components for stabilizing bio-analytes from the bio-sample (e.g., DNA, RNA, or protein), and (iii) optionally a separation component for separation of one or more sample components (e.g., plasma, or cells). A further component of the system or method disclosed herein can include kits, devices, methods, and systems for processing samples and analyzing user-provided samples.

Devices, systems, methods, and kits can include a sample acquisition component (SAC) for acquiring the sample, as well as a sample stabilization component (SSC) for transferring, collecting, and stabilizing the sample. In some instances the sample stabilization component can be further equipped to separate one or more components of the sample prior to transferring the sample to a solid substrate for stabilization. SACs can be easy to use, enabling un-trained professionals to collect samples at a variety of locations, from the clinic to a patient's home or office. SACs can even enable a donor to collect his or her own sample at home, without the need to visit a medical clinic or a dedicated point of collection where sample collection can be done by a trainer or un-trained professional.

Non-limiting embodiments can be directed towards integrated and non-integrated combinations of components for sample acquisition, sample separation and sample stabilization. Embodiments can include a single integrated device with distinct internal components for stabilizing components of the sample. Additional integrated devices can include units for separating components of the sample prior to selectively stabilizing sample components. Other embodiments can provide non-integrated components within a system or kit; components can include a sample acquisition component for acquiring the sample, and a separate sample collection component for stabilizing and optionally separating the sample. The sample stabilization component can include a solid stabilization matrix for selectively stabilizing and storing components of the sample, and it can also have a component for separating the sample prior to stabilization. In yet additional non-integrated systems or kits a sample stabilization component can be separate from the sample separation unit. Further embodiments can provide methods, devices, systems and kits for receiving, preparing, and/or treating the stabilized samples after the sample has been acquired, separated, components of the sample have been selectively stabilized.

### Sample Acquisition Component (SAC)

Devices, systems, methods and kits described herein can include one or more sample acquisition components (SACs). Samples acquired by the systems herein can be, e.g., any biological sample from an organisms such as blood, serum, urine, saliva, tissue, hair, skin cells, semen, or from the environment, e.g., water sample, oil from well, or from food, e.g., milk. Samples can be liquid, solid or a combination of liquids and solids.

Sample volumes can be fixed by components of the unit, including but not limited to the device collection chamber, materials properties of the collection system, sample settings pre-determined by the user, specifications established during device manufacturing, or any combination thereof.

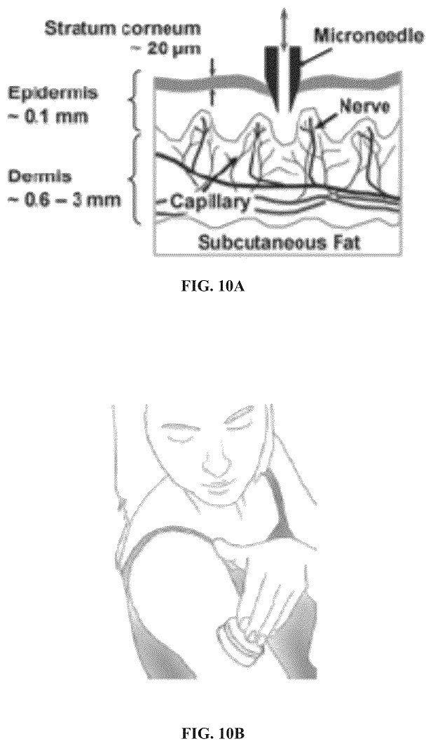

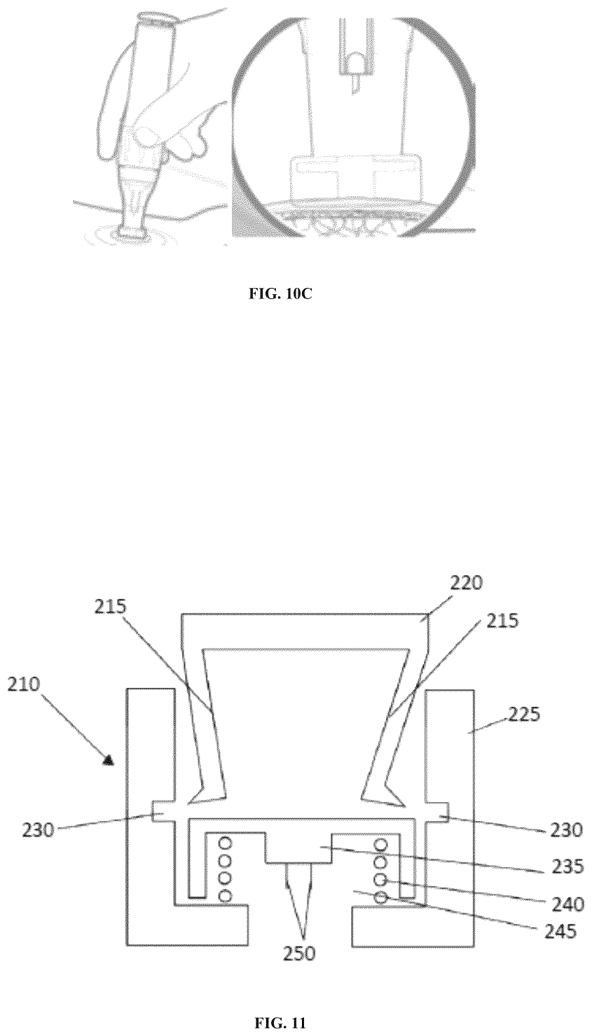

A SAC can include devices for venous blood draw or capillary blood draw. To accomplish this, the SAC can include one or more piercing elements. Piercing elements can be hollow or solid, and they can be configured for pain-free and efficient sample transfer; adaptations can involve use of materials with specific composition, surface microstructure, mechanical properties, structural shapes or combination thereof. Piercing elements can include needles, micro-needles, or lancets (including pressure activated needles or lancets). The SAC can optionally be configured to minimize physical pain or discomfort to the user. An SAC can preferably include micro-needle technology shown in FIG. 10A, which allows for shallow penetration of the skin to generate blood flow. Examples for sample, e.g., blood collection, contemplated herein include those described in U.S. Pat. No. 9,033,898. The SAC can be single use.

A SAC can collects a sample in the μL volume range (i.e., <1 mL). For example, the SAC can be configured to collect a small volume of blood e.g., less than or equal to 1 ml, 500 μL, 400 μL, 300 μL, 200 μL, 100 μL, 90 μL, 80 μL, 70 μL, 60 μL, 50 μL, 40 μL, 30 μL, 20 μL or 10 μL. In some embodiments, a SAC can be configured to collect larger samples of blood, for example samples of greater than or equal to 1 ml, 2 ml, 3 ml, 4 ml, 5 ml, 8 ml, 10 ml, 12 ml or 15 ml.

However some SACs can be used to collect samples in the mL volume range. Examples of SACs include a urine cup, a finger stick, or devices, such as those described in US. Pub. Nos. 20130211289 and 20150211967.

A SAC can be a component of an integrated device configured for collecting, stabilizing and storing samples. Or it can be a separate component that is part of a kit or a system. The SAC can include a vial for collecting the sample.

When a SAC is non-integrated but is part of a kit, the kit can include one or more of the following: (i) one or more sample separation units, (ii) one or more sample stabilization units, (iii) one or more bio-sample separation and/or stabilization components. A kit used for blood samples can comprise a capillary or transfer tube for collecting a blood drop from a lanced or incised finger and subsequently dispensing the blood onto a device or separate unit for stabilizing or separating and stabilized sample components.

Some embodiments of the sample acquisition component are illustrated in FIG. 10B, FIG. 10C and FIG. 11. As shown in FIG. 10B the SAC can be activated through mechanical means. Manual activation can be performed by the donor or end user. FIG. 10C illustrates an SAC that is activated with electrical means. The SAC, as shown, can uses vibration to induce blood flow.

The SAC can use a plunger to create a vacuum that drives the sample into one or more chambers in the device. As shown in FIG. 11, the sample acquisition component (SAC) can have a proximal end, a distal end, an outer surface and a lumen defined with the body 215. The base attached to the distal end of the body can comprise an outer face, an inner face and comprise one or more apertures 240. Additionally, the device can have a plunger 220 comprising a proximal and distal end, with the plunger configured to be user actuated. One or more piercing elements 250 can be fixed to the face of the plunger, and when the user actuates the SAC from the proximal end to the distal end of the device body, the piercing elements puncture the skin. A vacuum can be created upon withdrawal of the piercing elements. Blood can pool through the one or more apertures 240, and the apertures can be configured to draw the sample into the lumen. Sample can moves into the lumen and within the device, e.g., through spontaneous capillary-driven flow. Spontaneous capillary-driven flow can also be used to extract fluid from a pool or droplet on a surface, such as human skin, for a reservoir, or from another open microfluidic channel. Spontaneous capillary driven flow can be used to collect the sample from the lance site; it can also be used to manipulate fluids from a reservoir within the device or between sample collection and preparation steps using complex open microfluidic channels. In alternative embodiments, spontaneous capillary-driven flow can be combined with other means of moving sample through the device. The apertures can be optionally adapted for, or optionally contain materials or structures adapted to draw the sample from the penetrated skin into the device. For example in one embodiment, the piercing element can partially retract into the one or more apertures wherein the properties of the piercing element itself draw the sample through the aperture and into the sample chamber, either through the piercing element if it is hollow or on the sides of the piercing element it is solid. Embodiments of these components can be found in US. Pub. No. 20130211289.

Sample collection can occur from sample pooled at or above the skin surface, it can also optionally be collected from reservoirs under the skin. The SAC can, for example, create a lancing motion which cuts into the small but plentiful capillaries in the superficial vascular plexus under the epidermis e.g., at depth of 0.3-0.6 mm from the surface of the skin. This invention provides a system for mechanically massaging a lance site at other body locations by several different approaches, including oscillating an annular ring surrounding the wound to pump the blood surrounding the wound into the wound for extraction by a needle or capillary tube or oscillating paddles or other members adjacent the wound to achieve the desired blood flow. Further, bringing a drop of blood from the skin in other regions of the body, e.g., the thigh, to a small area on a test device is very difficult. An alternate embodiment of the present invention works with the needle remaining in the wound and the needle being mechanically manipulated to promote the formation of a sample of body fluid in the wound.

Liquid sample can collect or pool into a collection chamber, after the collection chamber or in lieu of a collection chamber the sample can optionally be absorbed through one or more particles, materials, structures or filters with optimized porosity and absorptivity for drawing the sample into the device. Materials for drawing the sample into the devices herein can consist of any absorptive or adsorptive surfaces, or materials with modified surfaces; optional materials including but not limited to paper-based media, gels, beads, membranes, polymer based matrices or any combination thereof. For example in one embodiment, the SAC can comprise a body that defines a fluid flow path from an inlet opening, wherein the flow path includes a bed of a porous polymer monolith selected to adsorb biological particles or analytes from a matrix drawn or dispensed through the inlet opening and the bed. The porous polymer monolith can absorb biological particles or analytes for later preparation steps. Examples of sample collection on a porous monolith can be found in US Pub. No. US20150211967.

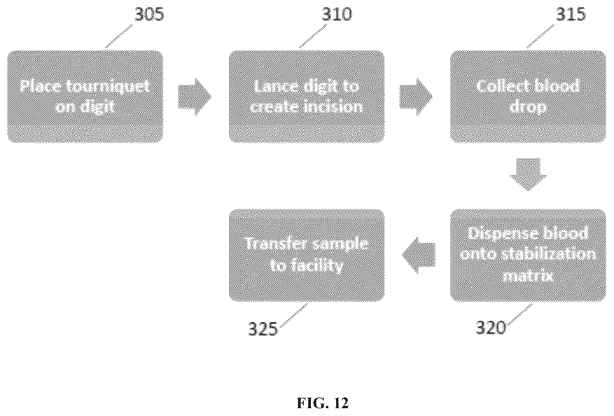

Methods for using the sample acquisition component (SAC) can include piercing the skin with an SAC followed by standard milking or squeezing of a finger to extract blood samples. The SAC can be used with a tourniquet or other components for facilitating sample acquisition. A tourniquet, rubber band, or elastic material can be placed around the first, second, or third digit of a subject's hand. Methods can be constructed to improve sample quality. As depicted in FIG. 12, steps can include placing a tourniquet on one of the digits of the donor's finger to apply pressure 305 and lancing the digit to create an incision 310. The method can also include optionally removing the first blood immediately after lancing while still applying pressure. Blood can be collected from the incision 315 by holding a capillary tube against a blood drop formed from the incision site. Blood collected in the capillary tube can be dispersed onto a separate component of the system 320, and the separate part of the component can be deployed to a different location or facility 325 for analysis. In certain embodiments the kit can include components and instructions for facilitating blood collection and efficiency. Methods can further comprise steps for preparing the hand and finger to insure proper blood flow including temperature and position, methods of applying the tourniquet to the finger, methods of sterilization, lancing, and actual blood collection. Methods for collecting blood samples are cited in U.S. application Ser. No. 14/450,585.

### Sample Stabilization Component

In some embodiments a sample can be acquired using the sample acquisition component prior to being transferred to a sample stabilization component (SSC). In other embodiments, a sample can be directly transferred to a SSC. A SSC can be any device or system that the sample is collected on or transferred to for stabilization and storage. The device or system can comprise channels and compartments for collecting and transferring the samples, and one or more units for separating and stabilizing sample.

A sample stabilization component (SSC) can collect a sample in the μL volume range (i.e., <1 mL). For example, the SSC can be configured to collect a small volume of blood e.g., under 1 mL, 500 μL, 400 μL, 300 μL, 200 μL, 100 μL, 90 μL, 80 μL, 70 μL, 60 μL, 50 μL, 40 μL, 30 μL, 20 μL or 10 μL. In other embodiments, the SSC can be configured to collect and stabilize components from a liquid sample with a volume of less than or equal to 1 ml, 2 ml 3 ml, 4 ml, 5m, 10 ml, 15 ml.

Samples stabilized by the SSC can be any biological sample from any organisms (e.g., blood, serum, urine, saliva, tissue, hair, skin cells, semen, cerebrospinal fluid) or from the environment (e.g., water sample, oil from a well) or from food, (e.g., milk). Samples can be liquid, solid or a combination of liquids and solids.

In a non-integrated system, the sample acquisition component can be physically separate from the sample stabilization component. In these embodiments, transfer from the sample acquisition component to the sample stabilization component can occur through a variety of means including one or more: needles, capillary tubes, or through direct transfer of the sample from the donor site to the sample stabilization component. In instances where the sample acquisition component is a separate component from the sample stabilization component, application of a sample to a substrate can be achieved using a self-filling capillary for collection from the sample acquisition component, followed by sample transfer to the substrate.

The sample stabilization component can be integrated with the SAC. Integration between the sample stabilization component and the SAC can occur through a shared assembled interface. Sample can move from the sample acquisition component through channels, including microchannels, spontaneous capillary flow, wicking through absorbent materials or other means that allow sample to flow through the sample stabilization component towards or into the substrate with minimal effort on behalf of the end user. Microchannels can be constructed from a variety of materials with properties including adapted shapes with surface microstructures and material properties adapted to facilitate capillary action or other means of sample transfer through the device. Microchannels can comprise any means of transferring sample between chambers, including open microfluidic channels optimized for moving samples using spontaneous capillary flow. Examples of microchannels and devices comprising microchannels can be found in many of the incorporated references, including US Pub. No. 20140038306.

The sample stabilization component can include a structure with multiple layers. It can also have an interface for accepting transfer to one or more layers or to a substrate. The sample stabilization component can be configured for collecting one or more samples, separating components of a sample, stabilizing one or more samples, or any combination thereof. The SSC can further comprise a network of layers and capillary channels configured for transferring sample between multiple layers and into substrate, as provided for by US Appl. Nos. U.S. Ser. No. 14/340,693 and U.S. Ser. No. 14/341,074.

The SSC can be coupled to the substrate in a variety of ways. The SSC can couple to the substrate in a way that enables contact with a layer for transferring the sample fluid from the integrated device to the substrate. The SSC can be configured such that the device is easily removable from the substrate. The system can further comprise a substrate frame having a region configured to receive the sample on the substrate. The substrate can be attached to the substrate frame in a way that makes it easy to remove the substrate from the system, and the substrate frame can comprise a barcode to enable automated or semi-automated processing. The system can further be coupled to an external device, wherein the external device comprises a fluidic device, an analytical instrument, or both. In one or more embodiments, the sample stabilization component can be coupled to a substrate, wherein SSC is configured to transfer the sample fluid to the substrate. The substrate can comprise the substrate or the sample separation component. The SSC can either be attached directly to the substrate or to a substrate frame that holds the substrate. In some embodiments, the SSC can further couple to a substrate frame and a substrate cover. The substrate frame and substrate cover can include features to facilitate efficient fluid transfer to the substrate at a region of interest, e.g., at the center of the substrate. In some embodiments, the SSC is packaged with a sample storage substrate, wherein the sample stabilization component is pre-attached to the sample substrate. In some other embodiments, the SSC and substrate are packaged separately, wherein the user can assemble the substrate and the SSC for sample collection, transfer, stabilization and storage. The SSC can be further packaged with a sample acquisition component (SAC).

A SSC can be disposable or re-usable. For example, an SSC can be a single-use disposable device configured to collect the sample and transfer the sample fluid to a substrate and facilitate loading of the fluid sample through desirable areas of the substrate. The SSC can be configured for one time use to reduce or prevent contamination or spreading of infection via the collected sample. The SSC can be configured for reliable and reproducible collection, transfer and storage of biological samples.

After collection and transfer of the biological sample, the substrate can be configured to separate bio-sample components prior to transferring to the stabilization matrix for storage.

### Sample Separation

The SSC can include a sample separation unit comprising one or more substrates, membranes, or filters for separating sample components. The sample separation unit can be integrated within the sample stabilization component, or it can be attached to or separate from the sample stabilization component.

Sample separation can occur at different points in the sample collection process. For example, in an integrated device sample separation can occur within the SSC, for non-integrated devices sample separation can occur outside of the SSC prior to transfer to the sample stabilization component. In other instances the sample can move through the SAC and into the sample separation unit before being transferred to the SSC which can transfer the separated sample to one or more substrates for stabilization and storage.

Sample separation can occur as an intermediate step between sample acquisition and transfer to a sample stabilization matrix. In some instances sample separation and stabilization can occur in one step without the need for user intervention. Sample separation can further occur sequentially or simultaneously with sample stabilization.

The sample acquisition and stabilization can require user action to proceed between one or more phases of the sample collection, optional separation, and stabilization process. An integrated device can require user action to activate sample acquisition, and move sample between separation, stabilization, storage. Alternatively, user action can be required to initiate sample acquisition as well as one or more additional steps of the sample collection, separation or stabilization process. User action can include any number of actions, including pushing a button, tapping, shaking, rupture of internal parts, turning or rotating components of the device, forcing sample through one or more chambers and any number of other mechanisms. Movement through the phases can occur in tandem with sample collection, or can occur after sample collection. Anytime during or prior to the processing phases the entire sample or components of the sample can be exposed to any number of techniques or treatment strategies for pre-treatment of cells of biological components of the sample; potential treatment includes but is not limited to treatment with reagents, detergents, evaporative techniques, mechanical stress or any combination thereof.

The devices, methods, systems and kits disclosed herein can comprise one or more sample separation units. Sample separation units can be used, e.g., to separate plasma from blood, cells from a water sample, or cells from cell free components. For blood samples one or more components can be used to separate plasma or specific cells from other components of a blood sample. Alternatively, separation devices, methods and systems can selectively separate any number of sample components including cells, plasma, platelets, specific cell types, DNA, RNA, protein, inorganic materials, drugs, or any other components.

Non-limiting embodiments of the sample stabilization unit can employ sample separation components to separate other non-plasma components as well. Sample separation components can be connected to the sample acquisition component e.g., through channels, including microchannels, wicking of absorbent materials or other means that allow sample to flow through the device. The systems and methods for separating the sample are exemplary and non-limiting.

There are many methods for performing separation, some of which use size, deformability, shape or any combination thereof. Separation can occur through one or more membranes, chambers, filters, polymers, or other materials. Membranes, substrates, filters and other components of the device can be chemically treated to selectively stabilize components, facilitate flow of sample, dry the sample, or any combination thereof. Alternative separation mechanisms can include liquid-liquid extraction, solid-liquid extraction, and selective precipitation of target or non-target elements, charge separation, binding affinity, or any combination thereof. Separation phase can be comprised of one or more steps, with each step relying on different mechanisms to separate the sample. One such mechanism can utilize size, shape or deformation to separate larger components from smaller ones. Cell separation can occur through a sorter that can for example rely on one or more filters or other size exclusion methods to separate components of the sample. Separation can also be conducted through selective binding wherein specific components are separated by binding events while the unbound eluant moves into or through alternate chambers.

In some methods, a single membrane can be used for separation and collection of one or more sample components from the bulk sample. Single membrane methods can use a device wherein samples can be applied to one end of the membrane and as the sample flows through a first component of the sample, for example cells, can be separated from a second component of the sample, for example plasma, based on the size of the membranes pores. After operation of the device the membrane containing the first component of the sample, cells in this example, can be severed from the portion containing the second component of the sample, plasma in this example, necessitating an additional step of severing the membranes. In another method, two separate membranes can be used for the separation and collection sample components; specifically, a first membrane for the separation of one component, for example blood cells, and a second membrane for collection of other components, for example plasma. These membranes can be arranged such that a distal end of the first membrane contacts a proximal end of the second membrane to facilitate the separation of a large component, for example cells, via the first membrane and the collection of a second smaller component, for example plasma, via the second membrane.

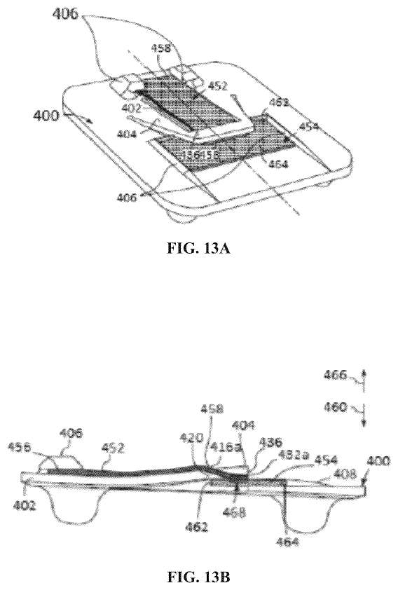

FIG. 13A and FIG. 13B illustrate a sample separation unit that can be used to separate samples prior to stabilization or in tandem with stabilization. Sample separation units can comprise a frame 400, a separation membrane 452, and a collection membrane 454. The frame can include an inner side disposed proximate to a first peripheral portion of the frame. The inner side 402 is formed from a plurality of first slots in the frame 416. The frame further includes an outer side 404 disposed surrounding at least a portion of the plurality of first slots 416. The outer side 404 is formed from a plurality of second slots in the frame 432. A distal end of the separation membrane 420 is disposed under the outer side, and a proximal end of the collection membrane is disposed under at least one of the outer side and inner side such that the proximal end of the collection membrane has an overlapping contact area 428 with the distal end of the separation membrane. Further, the outer side is configured to apply pressure on the separation and collection membranes about the overlapping contact area. The frame 400, separation membrane 452, and collection membrane 454, can be comprised of different materials. The frame 400 can be comprised of a polymer material such as polypropylene, nylon (polyamide), high density polyethylene (HDPE), and polyetheretherketone (PEEK); and it can be manufactured using an injection molding technique and has a uniform thickness. The separation membrane 452 can include suitable materials such as cellulose, a glass fiber, a cellulose acetate, a poly vinyl pyrrolidone, a polysulfone, a polyethersulfone, a polyester or combinations of these materials; and it can be configured to have a geometry compatible with the geometry of the frame 400, specifically, the geometry of the inner side 402 of the frame 400. The collection membrane 454 can include suitable materials such as cellulose, a glass fiber, a cellulose acetate, a poly vinyl pyrrolidone, a polysulfone, a polyethersulfone, polyester, or combinations of these materials; and the collection membrane can be chemically treated. Other embodiments and methods can include the step of displacing, such as by pressing downwards 460, an inner side of the frame 402 to insert a distal end 458 of a separation membrane 452 under the second distal end portion 436 of the outer side of the frame 404 via a first mid-slot 416b of the inner side 402. The method can further include the step of displacing the outer 404 and inner sides 402, for example by applying pressure by pushing upwards 466, to insert a proximal end 462 of a collection membrane 454 under at least one of the outer and the inner sides via a second mid-slot of the outer side 432b, such that the proximal end of the collection membrane has an overlapping contact area 468 with the distal end 458 of the separation membrane 452.

The separation machinery can be optional, for example it can be part of a modular system wherein the user or the manufacturer can insert a cartridge within the path of the sample. In one potential embodiment the sample can be transferred from any of the previously mentioned collection devices into a secondary chamber. The transfer can be facilitated by user action or it can happen spontaneously without user action.

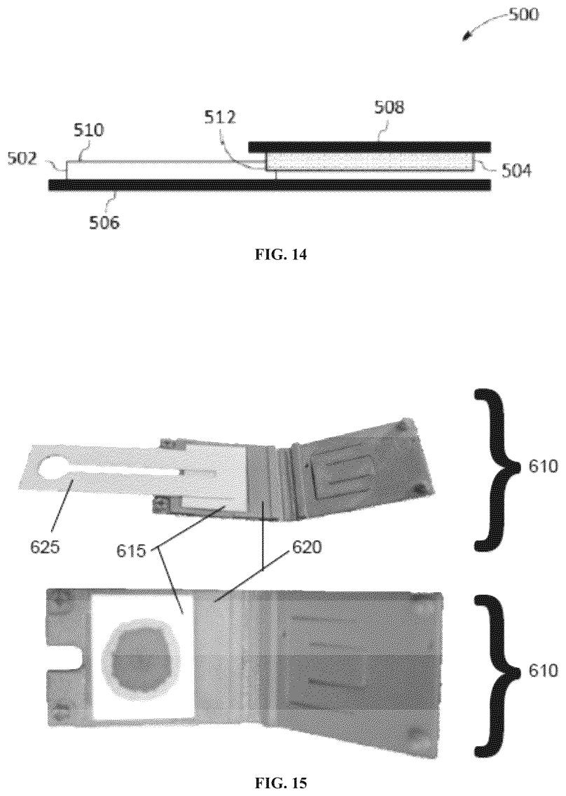

The sample separation unit can use a filtration membrane to separate sample components. FIG. 14 illustrates a sample separation unit with a filtration membrane that separates out the non-cellular fraction of a biological sample. The filtration membrane 502 has a sample application zone 510 and a transfer zone 512. The filtration membrane is in direct contact with a solid matrix 504 via the transfer zone 512. A biological sample is applied to the sample application zone 510 of the filtration membrane, and is filtered as it moves through the filtration membrane. The filtration membrane has a plurality of pores. Once the biological sample passes through the filtration membrane, resident intact cells within the biological sample are retained by the filtration membrane, mostly at the sample application zone 510 and the non-cellular fraction are passed through the pores to reach the transfer zone 512 and gets transferred and collected onto the dry solid matrix. A filtration membrane can a wide range for example pore sizes can range from 0.01 micron to about 5 micron.

Alternatively, the filtration membrane can have a narrow range of pore sizes, for example, between about 1 micron to about 2 micron. The pore size, can be on the lower size, for example it can vary between about 0.22 micron to about 2 microns. When a filtration membrane of 1 micron pore size is used, any other circulating eukaryotic cells and/or pathogenic cells having diameters greater than 1 micron will be retained in the filtration membrane and so will not reach the dry solid matrix upon filtration. Additional separation components are described in US Pub. No. 20150031035.

Filtration can occur at various points in the sample collection process. A non-cellular fraction of a sample can, for example, be filtered out from the biological sample at the point-of-collection itself. Filtration can be performed without any prior pre-treatment of the biological sample. Further filtration can be performed in absence of any stabilizing reagent.

Filtration membrane can be made from a variety of materials. The materials used to form the filtration membrane can be a natural material, a synthetic material, or a naturally occurring material that is synthetically modified. Suitable materials that can be used to make the filtration membrane include, but are not limited to, glass fiber, polyvinyl alcohol-bound glass fiber, polyethersulfone, polypropylene, polyvinylidene fluoride, polycarbonate, cellulose acetate, nitrocellulose, hydrophilic expanded poly(tetrafluoroethylene), anodic aluminum oxide, track-etched polycarbonate, electrospun nanofibers or polyvinylpyrrolidone. In one example, the filtration membrane is formed from polyvinyl alcohol-bound glass fiber filter (MF1™ membrane, GE Healthcare). In another example, filtration membrane is formed from asymmetric polyethersulfone (Vivid™, Pall Corporation). In some embodiments, filtration membrane can be formed by a combination of two or more different polymers. For example, filtration membrane can be formed by a combination of polyethersulfone and polyvinylpyrrolidone (Primecare™, iPOC).

After filtration, the separated, non-cellular fraction can be collected onto a dry solid matrix by means of physical interaction. The non-cellular fraction can be collected on to dry solid matrix by means of adsorption or absorption.

The sample stabilization component (SSC) can be used to transfer, stabilize, and store target components of a bio-sample which can comprise biologically sourced analytes such as nucleic acids, proteins, and respective fragments thereof. The SSC can receive, extract and stabilize one or more of these analytes onto a substrate that can be coupled to or housed within the sample stabilization component. FIG. 15 illustrates an example of a sample stabilization component 610 with a sample substrate or matrix 615 for preserving or stabilizing the sample. The substrate 615 is held in place on one side of the device using a frame 620. The sample is collected and transferred through a channel 625 to the mounted sample substrate.

### Concentration

A first volume of a sample can be applied to a solid support matrix, e.g., as described herein. The first volume can be the sample volume comprising an analyte to be concentrated.

The first volume can be 20 milliliters (mL) or less. The first volume can be about 10 μL. The first volume can be approximately any of the following: about 1000 mL, 100 mL, 50 mL, 20 mL, about 18 mL, about 15 mL, about 12 mL, about 10 mL, about 8 mL, about 5 mL, about 4 mL, about 3 mL, about 2 mL, about 1 mL, about 900 microliters (μL), about 800 μL, about 700 μL, about 600 μL, about 500 μL, about 400 μL, about 300 μL, about 200 μL, about 100, μL, about 90 μL, about 80 μL, about 70 μL, about 60 μL, about 50 μL, about 40 μL, about 30 μL, about 20 μL, or about 10 μL. The first volume can be less than 1000 mL, less than 100 mL, less than 50 mL, less than about 20 mL, less than about 18 mL, less than about 15 mL, less than about 12 mL, less than about 10 mL, less than about 8 mL, less than about 5 mL, less than about 4 mL, less than about 3 mL, less than about 2 mL, less than about 1 mL, less than about 900 microliters (μL), less than about 800 μL, less than about 700 μL, less than about 600 μL, less than about 500 μL, less than about 400 μL, less than about 300 μL, less than about 200 μL, less than about 100, μL, less than about 90 μL, less than about 80 μL, less than about 70 μL, less than about 60 μL, less than about 50 μL, less than about 40 μL, less than about 30 μL, less than about 20 μL, or less than about 10 μL. The first volume can be more than 1000 mL, more than 100 mL, more than 50 mL, more than about 20 mL, more than about 18 mL, more than about 15 mL, more than about 12 mL, more than about 10 mL, more than about 8 mL, more than about 5 mL, more about 4 mL, more about 3 mL, more about 2 mL, more about 1 mL, more about 900 microliters (μL), more than about 800 μL, more than about 700 μL, more than about 600 μL, more than about 500 μL, more than about 400 μL, more than about 300 μL, more than about 200 μL, more than about 100, μL, more than about 90 μL, more than about 80 μL, more than about 70 μL, more than about 60 μL, more than about 50 μL, more than about 40 μL, more than about 30 μL, more than about 20 μL, or more than about 10 μL.

The first volume can comprise an analyte to be concentrated. An analyte can be a protein or nucleic acid to be concentrated in the first volume applied to a solid support matrix. An analyte can be a non-concentrated sample component. An analyte can be a component of a biological sample. An analyte can be a specific molecule or class of molecules. An analyte can include a protein, a nucleic acid, an amino acid, a steroid, or an oligosaccharide. An analyte can be a molecule of therapeutic, diagnostic, research or otherwise analytic interest.

An analyte can be eluted from the solid support matrix using a second volume. The second volume can be less than the first volume, thereby concentrating the analyte. The second volume can be about 5% to about 95% less than the first volume. The second volume can be about 5%, about 10%, about 20%, about 25%, about 30%, about 35%, about 40%, about 45%, about 50%, about 55%, about 60%, about 65%, about 70%, about 75%, about 80%, about 85%, about 90%, about 95% less than the first volume. The second volume can be about, or more than 5%, 10%, 20%, 50%, 75%, 80%, 90%, or 95% less than the first volume.

The second volume can be an elution buffer. An elution buffer can be used to elute an analyte from the solid support matrix. An elution buffer can have a variety of properties that enable selective elution of an analyte from the solid support matrix using a second volume that is less than a first volume. An elution buffer can alter the degree of ionization on analyte, affinity molecule, or solid support matrix component. The degree of ionization can be altered by increasing or decreasing the pH or ionic strength of the elution buffer. Exemplary buffer salts include sodium chloride (NaCl), Tris-EDTA, zinc chloride (ZnCl₂), ammonium acetate (CH₃CO₂NH₄), BIS-TRIS propane, HEPES, magnesium acetate tetrahydrate, magnesium chloride (MgCl₂), magnesium sulfate (MgSO₄), potassium acetate (CH₃COOK), potassium chloride (KCl), sodium acetate (CH₃COONa), sodium citrate tribasic dehydrate, sodium phosphate dibasic. An elution buffer can also comprise a selective eluent. The selective eluent can effectuate competitive elution. One or more analytes can be selectively eluted by a concentration gradient of a single eluent or by pulse elution. An elution buffer can decrease the polarity of one or more analyte eluent(s). Chaotropic eluents can also be used to elute a stabilized analyte.

In other aspects, an affinity molecule can bind to an analyte. The analyte can then be eluted from or with the affinity molecule. An affinity molecule can bind a molecule, such as an analyte, with some degree of specificity. An affinity molecule can reversibly or non-covalently bind an analyte. An affinity molecule can bind an analyte specifically or non-specifically. Specific binding can refer to an affinity molecule targeted to binding a single analyte in a sample. For example, a monoclonal antibody can be used to bind a particular antigen on an analyte of interest. Non-specific binding can refer to the binding of an affinity molecule to multiple types of analytes or molecules. As a non-limiting example, non-specific binding can refer to an affinity molecule that binds antibody heavy chain, such as antibody heavy chain binding protein or an Fc receptor. Thus non-specific affinity molecule binds antibody heavy chain regardless of antibody binding site specificity.

An affinity molecule can be an antibody, a receptor, or other ligand binding molecule. An antibody can be a monoclonal antibody, a polyclonal antibody, and a trap antibody. An antibody can be selected from IgG, IgA, IgD, IgM, or IgE isotypes. An antibody can be selected from a recombinant antibody, a chimeric antibody, a humanized antibody, or a bispecific antibody. Antibody fragments can also be affinity molecules. Antibody fragments can include antigen binding fragments (Fab), single chain variable fragments (scFv), unibodies, miniaturized antibodies e.g. small modular immunopharmaceuticals (SMIPs) and various other antibody fragments. An affinity molecule can be a receptor. Non-limiting examples of receptors or ligand-binding molecules include Fc receptor, antibody heavy chain binding protein, a lectin, a DNA binding protein, heparin, a histone, and a carrier protein. Receptor ligands can comprise one or more analytes.

The solid support matrix can further comprise an affinity molecule stabilized therein. The affinity molecule can incorporated into the solid support matrix using a variety of techniques. An affinity molecule can be impregnated into the solid support matrix. A solid support can be impregnated by dipping, wetting, or saturating the solid support in a solution comprising an affinity molecule or other molecules that aid in solid support (e.g. melezitose). The impregnated solid support matrix can dried using, for example, line oven conveyors. In some instances, oligonucleotide affinity molecules or molecules to aid in solid support can be synthesized directly onto the solid support matrix. The solid support matrix can be impregnated with oligonucleotide sequences or probes using deposition. Various deposition techniques can be used, e.g. inkjet or positive displacement.

An affinity molecule can be applied to the solid support matrix after a first sample volume is applied. An analyte from a first volume of a sample can be first stabilized by the solid support matrix. An applied affinity molecule can then specifically or non-specifically bind one or more matrix-stabilized analytes.

Following application of a first volume of a sample to a solid support matrix, one or more non-analyte sample components can be washed from the solid support matrix. Washing can also follow application of a sample volume to a solid support matrix comprising an affinity molecule or following the application of an affinity molecule.

Washing can be accomplished using a washing solution. The washing solution can comprise de-ionized water, distilled water, and/or various wash buffers. A wash buffer can contain any one of the following: 2-Amino-2-hydroxymethyl-propane-1,3-diol (Tris), 2-(N-morpholino) ethanesulfonic acid (MES), 3-(N-morpholino) propanesulfonic acid (MOPS), citrate buffers, 4-(2-hydroxyethyl)-1-piperazineethanesulfonic acid (HEPES), phosphate buffers or combinations thereof, Tris-Hydrochloride (TrisHCl), MgCl₂, NaCl, dithiothreitol (DTT), KCl. A wash buffer can comprise Tris-based saline or phosphate-buffered saline (PBS). Washing can be repeated multiple times. For instance, following application of a first volume of sample to the solid support matrix, the matrix can be washed two or more times. In some instances, the matrix can be washed 2, 3, 4, 5, or 6 times.

A washing volume can be applied to the solid support matrix using a variety of techniques and instruments. Non-limiting examples of instruments for applying a washing volume include a single-channel pipette, a multi-channel pipette, a manifold dispenser, and an autowasher.

A concentrated analyte can be detected by applying various detection molecules. Detection molecules can be used to indicate the presence, absence or quantity of an analyte to be concentrated. Examples include enzyme-labeled antibodies, fluorescent molecule labeled antibodies, and radioactive antibody isotypes.

A solid support matrix can produce varying yields of a concentrated analyte, e.g. varying percentages of an analyte applied to the solid support matrix can be eluted off. The analyte yield can be greater than about 20%, 25%, 30%, 35%, 40%, 45%, 50%, 55%, 60%, 65%, 70%, 75%, 80%, 85%, 90%, 95%, or 99%. The analyte yield can be less than about 20%, 25%, 30%, 35%, 40%, 45%, 50%, 55%, 60%, 65%, 70%, 75%, 80%, 85%, 90%, 95%, or 99%. The analyte yield can range from about 20% to about 99%; from about 20% to about 95%; from about 30% to about 90%. The analyte yield can be 100%.

An analyte can be concentrated by several fold in an eluted volume. An analyte can be concentrated about 1.2, 1.4, 1.6, 1.8, 2.0, 2.5, 3.0, 3.5, 4.0, 4.5, 5.0, 5.5, 6.0, 6.5, 7.0, 7.5, 8.0, 8.5, 9.0, 9.5, 10, 50, 100, 500, 1000, 5000, 10,000, 100,000, 500,000, or 1,000,000 fold. An analyte can be concentrated greater than about 1.2, 1.4, 1.6, 1.8, 2.0, 2.5, 3.0, 3.5, 4.0, 4.5, 5.0, 5.5, 6.0, 6.5, 7.0, 7.5, 8.0, 8.5, 9.0, 9.5, 10, 50, 100, 500, 1000, 5000, 10,000, 100,000, 500,000, or 1,000,000 fold. An analyte can be concentrated less than about 1.2, 1.4, 1.6, 1.8, 2.0, 2.5, 3.0, 3.5, 4.0, 4.5, 5.0, 5.5, 6.0, 6.5, 7.0, 7.5, 8.0, 8.5, 9.0, 9.5, 10, 50, 100, 500, 1000, 5000, 10,000, 100,000, 500,000, or 1,000,000 fold. The analyte can be concentrated about 2 fold to about 10 fold, about 5 fold to about 50 fold, about 10 fold to about 100 fold, or about 50 fold to about 500 fold.

A concentrated analyte, e.g., an enzyme, can be stabilized such that it has remaining activity. A concentrated analyte can have about 20%, 30%, 40%, 50%, 60%, 70%, 80%, 90%, or 99% activity of the analyte before concentration. A concentrated analyte can have greater than about 20%, 30%, 40%, 50%, 60%, 70%, 80%, 90%, or 99% activity of the analyte before concentration. A concentrated analyte can have less than about 20%, 30%, 40%, 50%, 60%, 70%, 80%, 90%, or 99% activity of the analyte before concentration. A concentrated analyte can have about 20% to about 99%, about 20% to about 95%, about 30% to about 90%, or about 30% to about 70% activity of the analyte before concentration.

The concentrated analyte can be used for various purposes, e.g. diagnostic and/or research purposes, in downstream applications. Downstream applications utilizing a concentrated analyte can include various protein or nucleic acid assays. Protein assays can include enzyme-linked immunosorbent assay (ELISA); mass spectrometry; 2-D gel electrophoresis; liquid chromatography, e.g. high-performance liquid chromatography; and others recognized by one having skill in the art. Nucleic acid assays can include sequencing (e.g., next-generation sequencing, e.g., nanopore sequencing, ion semiconductor sequencing, sequencing using reversible dye terminators and bridge amplification), microarrays, polymerase chain reaction (PCR), qPCR, cloning, among others recognized by one having skill in the art.

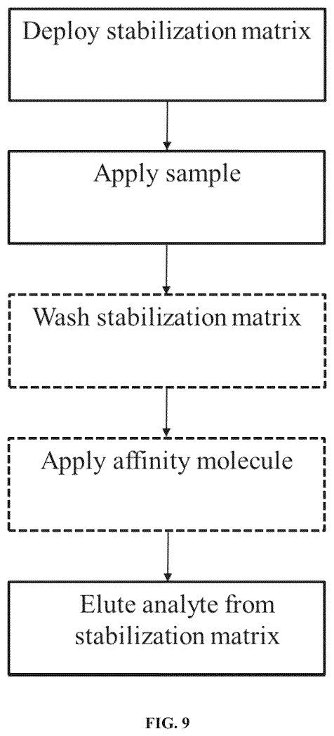

An exemplary embodiment is illustrated in FIG. 9. FIG. 9 generally illustrates a method for concentrating an analyte within a sample using a solid support matrix and method described herein. A sample is applied in a first volume to the dry solid support matrix. The solid support matrix can then be optionally washed to remove sample components that can not be adsorbed to the solid support matrix. An affinity molecule can optionally be applied to the solid support matrix, or an affinity molecule can be stabilized within the solid support matrix. Analyte is eluted off of the solid support matrix using a second volume, which is less than the first volume, thereby concentrating the analyte.

In some instances, the concentrated sample can comprise at least one protein or other sensitive biomolecule. In some cases, the solid matrix can selectively stabilize blood plasma components. Plasma components can include cell-free DNA, cell-free RNA, protein, hormones, and other metabolites, which can be selectively stabilized on the solid matrix. Plasma components can be isolated from whole blood and stabilized on a solid matrix. A solid matrix can be overlapping with or a component of a variety of different devices and techniques. Plasma components can be separated from whole blood samples using a variety of different devices and techniques. Techniques can include lateral flow assays, vertical flow assays, and centrifugation.

A solid matrix can be integrated with or a component of a variety of plasma separation devices or techniques. A solid matrix can be overlapping with or a component of a variety of different devices, such as a plasma separation membrane for example Vivid™ plasma separation membrane. A solid matrix can partially overlap with plasma separation device such as a plasma separation membrane. Examples of devices and techniques for plasma separation are disclosed in patents or patent publications, herein incorporated by reference, including U.S. Pat. Nos. 6,045,899; 5,906,742; 6,565,782; 7,125,493; 6,939,468; EP 0,946,354; EP 0,846,024; U.S. Pat. Nos. 6,440,306; 6,110,369; 5,979,670; 5,846,422; 6,277,281; EP 1,118,377; EP 0,696,935; EP 1,089,077, US 20130210078, US 20150031035.

In various devices and techniques, a separation membrane can be used. The separation membrane can comprise polycarbonate, glass fiber, or others recognized by one having skill in the art. Membranes can comprise a solid matrix. Membranes can have variable pore sizes. Separation membranes can have pore diameters of about 1 μm, about 2 μm, about 4 μm, about 6 μm, about 8 μm, about 10 μm, about 12 μm, about 14 μm, about 16 μm, about 18 μm, or about 20 μm. A separation membrane can have pores with diameters of about 2 μm to about 4 μm. A separation membrane can have pores that are about 2 μm in diameter.

Plasma separation can be implemented for a wide variety of sample volumes. Plasma sample volumes can be variable depending on the application for which a solid matrix is used. Sample volumes can be greater than about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL. Sample volumes can range from about 250 μL to about 500 μL.

### Elution of Nucleic Acids

In some cases, nucleic acids, e.g., DNA or RNA, in a sample (e.g., a biological sample), are applied to a stabilization matrix (e.g., nucleic acid stabilization matrix), the sample is optionally dried on the stabilization matrix, and the nucleic acid on the stabilization matrix, e.g., DNA or RNA, are eluted from the stabilization matrix. In some instances, a dried biological sample is stabilized on a stabilization matrix capable of stabilizing a nucleic acid, e.g., as described herein. In some instances, a method comprises the steps of: (a) contacting, e.g., spotting a sample, e.g., a biological sample, comprising a nucleic acid, e.g., DNA or RNA, on a nucleic acid stabilization matrix, (b) optionally drying the sample, e.g., biological sample, on the nucleic acid stabilization matrix, (c) optionally contacting the nucleic acid stabilization matrix comprising nucleic acid with a lysis buffer, (d) optionally contacting the nucleic acid stabilization matrix comprising nucleic acid with a nucleic acid binding buffer (optionally containing an organic solvent), e.g., by submerging the nucleic acid stabilization matrix comprising nucleic acid in the binding buffer; (e) optionally contacting the nucleic acid stabilization matrix comprising nucleic acid with a wash buffer, e.g., by submerging the nucleic acid stabilization matrix in the wash buffer, and (f) contacting the nucleic acid stabilization matrix comprising nucleic acid with an elution buffer, e.g., by submerging the nucleic acid stabilization matrix comprising nucleic acid in the elution buffer, to elute the nucleic acid from the stabilization matrix.

The elution can be performed on at least a portion of the stabilization matrix comprising a sample, e.g., a dried biological sample. In some cases, a portion of the stabilization matrix can be separated from the rest of the stabilization matrix, e.g., a portion of a stabilization matrix can be punched out of the stabilization matrix, and nucleic acids in the separated portion can be eluted. The punches can be about 0.5, 1, 2, 3, 4, 5, 6, 7, 8, 9, 10, 15, 20, 25, 30, 35, 40, 45, 50, 55, or 60 mm in diameter. The punches can be from about 10 to about 60, from about 10 to about 50, from about 10 to about 40, from about 10 to about 30, from about 10 to about 20, from about 1 to about 10, from about 2 to about 9, from about 3 to about 8, from about 4 to about 7, from about 5 to about 6, from about 3 to about 6, from about 1 to about 4, from about 1 to about 3, or from about 1 to about 2 mm in diameter. In some cases, a stabilization matrix comprising nucleic acid is not separated into portions before the nucleic acid is eluted from the stabilization matrix.

In some cases, a nucleic acid stabilization matrix comprising nucleic acid can be contacted with a lysis buffer. In some cases, the binding and retention of a nucleic acid to a stabilization matrix can be enhanced through contacting the stabilization matrix with a binding buffer. In some cases, a portion of the stabilization matrix is contacted with a nucleic acid binding buffer. In some instances, the nucleic acid binding buffer can comprise beads. In some cases, the stabilization matrix is contacted with a wash buffer, e.g., to remove impurities. In some instances, the nucleic acid can be eluted by contacting the stabilization matrix with an elution buffer.

In some instances, the nucleic acid lysis buffer, binding buffer, wash buffer, or elution buffer can comprise a commercially available buffer. For instance, a buffer can comprise TRIzol® manufactured by Thermofisher®, Buffer RLT manufactured by Qiagen®, Buffer RLN manufactured by Qiagen®, RNA Lysis Buffer (RLA) manufactured by Promega, PureYield™ Cell Lysis Solution (CLA) manufactured by Promega, PureYield™ Endotoxin Removal Wash manufactured by Promega, PureZOL™ RNA isolation reagent (Bio-Rad™), RNA Lysis Buffer or DNA/RNA Binding Buffer manufactured by Zymo Research Corp, or RNA Capture Buffer manufactured by Pierce™.

In some instances, the nucleic acid lysis buffer, binding buffer, wash buffer, or elution buffer can comprise one or more buffering agents (or pH buffer), one or more salts, one or more reducing agents, one or more chelators, one or more surfactants, one or more enzymes, one or more protein denaturants, one or more blocking reagents, or any combination thereof.

The one or more buffering agents can be saline, citrate, phosphate, phosphate buffered saline, acetate, glycine, tris(hydroxymethyl)aminomethane (tris) hydrochloride, tris buffered saline (TBS), 3-[[1,3-dihydroxy-2-(hydroxymethyl)propan-2-yl]amino]propane-1-sulfonic acid (TAPS), bicine, tricine, 3-[[1,3-dihydroxy-2-(hydroxymethyl)propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid) (PIPES), 3-(N-morpholino)propanesulfonic acid (MOPS), 2-(N-morpholino)ethanesulfonic acid (MES), 2-[[1,3-dihydroxy-2-(hydroxymethyl)propan-2-yl]amino]ethanesulfonic acid (TES), cacodylate, glycine, carbonate, or any combination thereof. The one or more buffering agents can be present at a concentration of from about 0.1 mM to about 500, from about 0.1 mM to about 400 mM, from about 0.1 mM to about 300 mM, from about 0.1 mM to about 200 mM, from about 0.1 mM to about 100 mM, from about 0.1 mM to about 50 mM, from about 0.1 mM to about 25 mM, from about 0.1 mM to about 20 mM, from about 0.1 mM to about 15 mM, from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.1 mM to about 0.9 mM, from about 0.1 mM to about 0.8 mM, from about 0.1 mM to about 0.7 mM, from about 0.1 mM to about 0.6 mM, from about 0.1 mM to about 0.5 mM, from about 0.1 mM to about 0.4 mM, from about 0.1 mM to about 0.3 mM, or from about 0.1 mM to about 0.2 mM. The buffering agent can be present at a concentration of less than 500 mM, less than 400 mM, less than 300 mM, less than 200 mM, less than 100 mM, less than 50 mM, less than 25 mM, less than 20 mM, less than 15 mM, less than 10 mM, less than 5 mM, less than 4 mM, less than 3 mM, less than 2 mM, less than 1 mM, less than 0.9 mM, less than 0.8 mM, less than 0.7 mM, less than 0.6 mM, less than 0.5 mM, less than 0.4 mM, less than 0.3 mM, less than 0.2 mM, or less than 0.1 mM. The buffering agent can be present at a concentration of more than 500 mM, more than 400 mM, more than 300 mM, more than 200 mM, more than 100 mM, more than 50 mM, more than 25 mM, more than 20 mM, more than 15 mM, more than 10 mM, more than 5 mM, more than 4 mM, more than 3 mM, more than 2 mM, more than 1 mM, more than 0.9 mM, more than 0.8 mM, more than 0.7 mM, more than 0.6 mM, more than 0.5 mM, more than 0.4 mM, more than 0.3 mM, more than 0.2 mM, or more than 0.1 mM.

The one or more salts can be sodium chloride, sodium acetate, sodium bicarbonate, sodium bisulfate, sodium bromide, potassium chloride, potassium acetate, potassium bicarbonate, potassium bisulfate, potassium bromate, potassium bromide, or potassium carbonate. The one or more salts can be at a concentration of about 0.1 mM, 5 mM, 10 mM, 25 mM, 50 mM, 100 mM, 250 mM, 500 mM, 750 mM, or 1000 mM. The one or more salts can be at a concentration of less than 0.1 mM, 5 mM, 10 mM, 25 mM, 50 mM, 100 mM, 250 mM, 500 mM, 750 mM, or 1000 mM. The one or more salts can be at a concentration of at least 0.1 mM, 5 mM, 10 mM, 25 mM, 50 mM, 100 mM, 250 mM, 500 mM, 750 mM, or 1000 mM.

The one or more reducing agents can be beta-mercaptoethanol (BME), 2-aminoethanethiol (2MEA-HCl (cysteamine-HCl)), dithiothreitol (DTT), glutathione (GSH), tris(2-carboxyethyl)phosphine (TECP), or any combination thereof. The concentration of the one or more reducing agents can be about 0.1 mM, 0.5 mM, 1 mM, 10 mM, 50 mM, 100 mM, 250 mM, or 500 mM. The concentration of the one or more reducing agents can be less than 0.5 mM, 1 mM, 10 mM, 50 mM, 100 mM, 250 mM, or 500 mM. For example, the concentration of DTT can be from about 0.05 mM to about 100 mM, from about 0.5 mM to about 50 mM, or from about 5 mM to about 10 mM. The concentration of TCEP can be from about 0.05 mM to about 50 mM, from about 0.5 mM to about 50 mM, or from about 0.5 mM to about 5 mM. The concentration of BME can be from about 0.05% to about 10%, from about 0.5% to about 5%, or from about 1% to about 10%. The concentration of GSH can be from about 0.05 mM to about 25 mM, from about 0.5 mM to about 10 mM, or from about 5 mM to about 10 mM, The concentration of the one or more reducing agents can be about 1 mM, 10 mM, 50 mM, 100 mM, 250 mM, or 500 mM.

The one or more chelators can be a carbohydrate; a lipid; a steroid; an amino acid or related compound; a phosphate; a nucleotide; a tetrapyrrol; a ferrioxamines; an ionophor; a phenolic; or a synthetic chelator such as 2,2′-bipyridyl, dimercaptopropanol, ethylenediaminotetraacetic acid (EDTA), ethylenedioxy-diethylene-dinitrilo-tetraacetic acid, ethylene glycol-bis-(2-aminoethyl)-N,N,N′, N′-tetraacetic acid (EGTA), a metal nitrilotriacetic acid (NTA), salicylic acid, or triethanolamine (TEA). The concentration of the one or more chelating agents can be about 0.1 mM, 1 mM, 5 mM, 10 mM, 20 mM, or 25 mM. The concentration of the chelating agent can be less than 0.1 mM, 1 mM, 5 mM, 10 mM, 20 mM, or 25 mM. The concentration of the chelating agent can be more than 0.1 mM, 1 mM, 5 mM, 10 mM, 20 mM, or 25 mM.

The one or more surfactants can be an anionic, cationic, nonionic or amphoteric type. The one or more surfactants can be polyethoxylated alcohols; polyoxyethylene sorbitan; octoxynol such as Triton X 100™ (polyethylene glycol p-(1,1,3,3-tetramethylbutyl)-phenyl ether); polysorbates such as Tween™ 20 ((e.g., polysorbate 20) or Tween™ 80 (polysorbate 80); sodium dodecyl sulfate; sodium lauryl sulfate; nonylphenol ethoxylate such as Tergitol™; cyclodextrins; or any combination thereof. The one or more surfactants can be present at a concentration of less than 0.001%, less than 0.005%, less than 0.01%, less than 0.015%, less than 0.02%, less than 0.025%, less than 0.03%, less than 0.035%, less than 0.04%, less than 0.045%, less than 0.05%, less than 0.055%, less than 0.06%, less than 0.065%, less than 0.07%, less than 0.075%, less than 0.08%, less than 0.085%, less than 0.09%, less than 0.095%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, or less than 0.1% by volume relative to the total volume of the elution buffer. The one or more surfactants can be at a concentration of about 0.01%, 0.05%, 0.1%, 0.5%, 1%, 5%, or 10%. The one or more surfactants can be at a concentration of less than 0.01%, 0.05%, 0.1%, 0.5%, 1%, 5%, or 10%. The one or more surfactants can be at a concentration of more than 0.01%, 0.050%, 0.10%, 0.50%, 10%, 50%, or 10%.

The one or more protein denaturants can be a chaotropic agent, e.g., a chaotropic salt. A chaotropic agent can be butanol, ethanol, guanidine chloride, guanidine hydrochloride, guanidine isothiocyanate, lithium perchlorate, lithium acetate, magnesium chloride, phenol, propanol, sodium iodide, sodium thiocyanate, thiourea, urea, or any combination thereof. The concentration of the chaotropic agent can be about 0.1 mM, 1 mM, 10 mM, 100 mM, 1 M, 6 M, or 8 M. The concentration of the chaotropic agent can be at least 0.1 mM, 1 mM, 10 mM, 100 mM, 1 M, 6 M, or 8 M. The concentration of the chaotropic agent can be less than 0.1 mM, 1 mM, 10 mM, 100 mM, 1 M, 6 M, or 8 M.

The nucleic acid lysis buffer, binding buffer, wash buffer or elution buffer can further comprise one or more enzymes. A lysis buffer, binding buffer, wash buffer or elution buffer can comprise DNase or RNase in amounts sufficient to remove DNA or RNA impurities, respectively, from each other. A lysis buffer, binding buffer, wash buffer or elution buffer can comprise lysis enzymes such as hen egg white lysozyme, T4 lysozyme and the like, as well as enzymes such as carbohydrases, phytases, and proteases such as trypsin, Proteinase K, pepsin, chymotrypsin, papain, bromelain, subtilisin, or elastase, A protease can be a serine protease, a cysteine protease, a threonine protease, an aspartic protease, a glutamic protease, or a metalloprotease, or an asparagine peptide lyase.

The nucleic acid lysis buffer, binding buffer, wash buffer or elution buffer can be an aqueous solution having a pH from about 2 to about 10, from about 2 to about 9, from about 2 to about 8, from about 2 to about 7, from about 2 to about 6, from about 2 to about 5, from about 2 to about 4, or from about 2 to about 3. The nucleic acid lysis buffer, binding buffer, wash buffer or elution buffer can be an aqueous solution having a pH from about 6 to about 8, from about 6 to about 7.9, between about 6 to about 7.8, between about 6 to about 7.7, between about 6 to about 7.6, between about 6 to about 7.5, between about 6 to about 7.4, between about 6 to about 7.3, from about 6 to about 7.2, from about 6 to about 7.1, from about 6 to about 7, from about 6 to about 6.9, from about 6 to about 6.8, from about 6 to about 6.7, from about 6 to about 6.6, from about 6 to about 6.5, from about 6 to about 6.4, from about 6 to about 6.3, from about 6 to about 6.2, or from about 6 to about 6.1. The nucleic acid lysis buffer, binding buffer, wash buffer or elution buffer can be an aqueous solution having a pH of at least about 3, at least about 4, at least about 5, at least about 6, at least about 7, at least about 8, at least about 9, or at least about 10.

The nucleic acid lysis buffer, binding buffer, wash buffer, or elution buffer can further comprise additional ingredients. For example, a lysis buffer, binding buffer, wash buffer, or elution buffer can comprise a protein blocking agent that minimizes non-specific binding such as bovine serum albumin, fetal bovine serum, and the like. A lysis buffer, binding buffer, wash buffer, or elution buffer can comprise additional nucleic acids (to include DNA or RNA) from an organism distinct from the subject such as bacterial DNA, bacterial DNA, yeast DNA, yeast RNA, mammalian nucleic acids including primate nucleic acids such as human or chimpanzee DNA, or non-mammalian nucleic acids including nucleic acids from fish such as herring, mackerel, krill, or salmon DNA and the like.

A lysis buffer, binding buffer, wash buffer, or elution buffer can comprise an amount of one or more organic solvents, e.g., to enhance binding of a nucleic acid to the stabilization matrix. The one or more organic solvents can be methanol, ethanol, DMSO, DMF, dioxane, tetrahydrofuran, propanol, isopropanol, butanol, t-butanol, or pentanol, acetone and the like. In some instances, a binding buffer can comprise less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 99%, or 100% of an organic solvent by volume relative to the total volume of the solution. The organic solvent can be at a concentration of at least 0.1%, 1%, 10%, 50%, 75%, or 100%. The organic solvent can be at a concentration of about 0.1%, 1%, 10%, 50%, 75%, or 100%.

The stabilization matrix, or the portion of the stabilization matrix, can be contacted with a volume of the nucleic acid binding buffer, wash buffer or elution buffer of less than 5 μL, less than 10 μL, less than 15 μL, less than 20 μL, less than 25 μL, less than 30 μL, less than 35 μL, less than 40 μL, less than 45 μL, less than 50 μL, less than 55 μL, less than 60 μL, less than 65 μL, less than 70 μL, less than 75 μL, less than 80 μL, less than 85 μL, less than 90 μL, less than 95 μL, less than 100 μL, less than 110 μL, less than 120 μL, less than 130 μL, less than 140 μL, less than 150 μL, less than 160 μL, less than 170 μL, less than 180 μL, less than 190 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1,000 μL, less than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, or less than 10 mL. The stabilization matrix, or portion of the stabilization matrix, can be contacted with about 0.1 mL, 0.2 mL, 0.5 mL, 0.7 mL, 1 mL, 2 mL, 5 mL, 7 mL, or 10 mL of buffer. The stabilization matrix, or portion of the stabilization matrix, can be contacted with at least 0.1 mL, 0.2 mL, 0.5 mL, 0.7 mL, 1 mL, 2 mL, 5 mL, 7 mL, or 10 mL of buffer.

The volume of binding buffer, wash buffer, or elution buffer contacted with the stabilization matrix can be dependent on the surface area of the stabilization matrix. The amount of binding buffer, wash buffer, or elution buffer can be less than 1 μL/mm², less than 2 μL/mm², less than 3 μL/mm², less than 4 μL/mm², less than 5 μL/mm², less than 6 μL/mm², less than 7 μL/mm², less than 8 μL/mm², less than 9 μL/mm², less than 10 μL/mm², less than 12 μL/mm², less than 14 μL/mm², less than 16 μL/mm², less than 18 μL/mm², less than 20 μL/mm², less than 25 μL/mm², less than 30 μL/mm², less than 35 μL/mm², less than 40 μL/mm², less than 45 μL/mm², less than 50 μL/mm², less than 55 μL/mm², less than 60 μL/mm², less than 65 μL/mm², less than 70 μL/mm², less than 75 μL/mm², less than 80 μL/mm², less than 85 μL/mm², less than 90 μL/mm², less than 95 μL/mm², less than 100 μL/mm², less than 150 μL/mm², less than 200 μL/mm², less than 250 μL/mm², less than 300 μL/mm², less than 350 μL/mm², less than 400 μL/mm², less than 450 μL/mm², less than 500 μL/mm², less than 550 μL/mm², less than 600 μL/mm², less than 650 μL/mm², less than 700 μL/mm², less than 750 μL/mm², less than 800 μL/mm², less than 850 μL/mm², less than 900 μL/mm², less than 950 μL/mm², or less than 1,000 μL/mm². In some cases, the amount of binding buffer, wash buffer, or elution buffer can be from about 10 μL/mm² to about 1,000 μL/mm², from about 10 μL/mm² to about 900 μL/mm², from about 10 μL/mm² to about 800 μL/mm², from about 10 μL/mm² to about 700 μL/mm², from about 10 μL/mm² to about 600 μL/mm², from about 10 μL/mm² to about 500 μL/mm², from about 10 μL/mm² to about 400 μL/mm², from about 10 μL/mm² to about 300 μL/mm², from about 10 μL/mm² to about 200 μL/mm², from about 10 μL/mm² to about 100 μL/mm², from about 10 μL/mm² to about 90 μL/mm², from about 10 μL/mm² to about 80 μL/mm², from about 10 μL/mm² to about 70 μL/mm², from about 10 μL/mm² to about 60 μL/mm², from about 10 μL/mm² to about 50 μL/mm², from about 10 μL/mm² to about 40 μL/mm², from about 10 μL/mm² to about 30 μL/mm², or from about 10 μL/mm² to about 20 μL/mm².

The lysis, binding, washing or elution of the nucleic acid can be performed in the presence or absence of agitation from an agitation source. An agitation source can be a rocker, vortexer, mixer, shaker and the like. In some cases, an agitation source can be set to a constant speed. The speed can be less than 1 rotations per minute (rpm), less than 5 rpm, less than 10 rpm, less than 15 rpm, less than 20 rpm, less than 25 rpm, less than 30 rpm, less than 35 rpm, less than 40 rpm, less than 45 rpm, less than 50 rpm, less than 55 rpm, less than 60 rpm, less than 65 rpm, less than 70 rpm, less than 75 rpm, less than 80 rpm, less than 85 rpm, less than 90 rpm, less than 95 rpm, less than 100 rpm, less than 150 rpm, less than 200 rpm, less than 250 rpm, less than 300 rpm, less than 350 rpm, less than 400 rpm, less than 450 rpm, less than 500 rpm, less than 550 rpm, less than 600 rpm, less than 650 rpm, less than 700 rpm, less than 750 rpm, less than 800 rpm, less than 850 rpm, less than 900 rpm, less than 950 rpm, less than 1,000 rpm, less than 1,500 rpm, less than 2,000 rpm, less than 2,500 rpm, less than 3,000 rpm, less than 3,500 rpm, less than 4,000 rpm, less than 4,500 rpm, less than 5,000 rpm, less than 5,500 rpm, less than 6,000 rpm, less than 6,500 rpm, less than 7,000 rpm, less than 7,500 rpm, less than 8,000 rpm, less than 8,500 rpm, less than 9,000 rpm, less than 9,500 rpm, or less than 10,000 rpm. The speed can be about 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm. The speed can be at least 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm.

The binding, washing or elution can be performed at a temperature of about 0° C., less than 1° C., less than 2° C., less than 3° C., less than 4° C., less than 5° C., less than 6° C., less than 7° C., less than 8° C., less than 9° C., less than 10° C., less than 11° C., less than 12° C., less than 13° C., less than 14° C., less than 15° C., less than 16° C., less than 17° C., less than 18° C., less than 19° C., less than 20° C., less than 21° C., less than 22° C., less than 23° C., less than 24° C., less than 25° C., less than 26° C., less than 27° C., less than 28° C., less than 29° C., less than 30° C., less than 31° C., less than 32° C., less than 33° C., less than 34° C., less than 35° C., less than 36° C., less than 37° C., less than 38° C., less than 39° C., less than 40° C., less than 45° C., less than 50° C., less than 55° C., less than 60° C., less than 65° C., less than 70° C., less than 75° C., less than 80° C., less than 85° C., less than 90° C., less than 95° C., or about 100° C. In some cases, the binding, washing or elution can be performed at a temperature of from about 10° C. to about 100° C., from about 10° C. to about 95° C., from about 10° C. to about 90° C., from about 10° C. to about 85° C., from about 10° C. to about 80° C., from about 10° C. to about 75° C., from about 10° C. to about 70° C., from about 10° C. to about 65° C., from about 10° C. to about 60° C., from about 10° C. to about 55° C., or from about 10° C. to about 50° C. In some cases, the lysis, binding, washing or elution can be performed at a temperature of from about 20° C. to about 50° C., from about 20° C. to about 48° C., from about 20° C. to about 46° C., from about 20° C. to about 44° C., from about 20° C. to about 42° C., from about 20° C. to about 40° C., from about 20° C. to about 38° C., from about 20° C. to about 36° C., from about 20° C. to about 34° C., from about 20° C. to about 32° C., from about 20° C. to about 30° C., from about 20° C. to about 28° C., from about 20° C. to about 26° C., from about 20° C. to about 24° C., or from about 20° C. to about 22° C. The temperature can be about 10° C., 20° C., 25° C., 30° C., 37° C., 50° C., or 65° C.

The lysis, binding, washing or elution can be performed for less than 1, less than 5, less than 10, less than 15, less than 20, less than 25, less than 30, less than 35, less than 40, less than 45, less than 50, less than 55, or less than 60 minutes. The binding, washing or elution can be performed for less than 0.5, less than 1, less than 1.5, less than 2, less than 2.5, less than 3, less than 3.5, less than 4, less than 4.5, less than 5, less than 5.5, less than 6, less than 6.5, less than 7, less than 7.5, less than 8, less than 8.5, less than 9, less than 9.5, less than 10, less than 10.5, less than 11, less than 11.5, or less than 12 hours, less than 18 hrs, less than 1 day, less than 1.5 days, less than 2 days, less than 2.5 days, less than 3 days, less than 3.5 days, less than 4 days, less than 4.5 days, less than 5 days, less than 5.5 days, less than 6 days, less than 6.5 days, or less than 7 days. The lysis, binding, washing, or elution can be performed for about 0.25 hr, 0.5 hr, 1 hr, 2 hr, 5 hr, 10 hr, 12 hr, 18 hr, 24 hr, 3 days, or 1 week. The lysis, binding, washing, or elution can be performed for at least 0.25 hr, 0.5 hr, 1 hr, 2 hr, 5 hr, 10 hr, 12 hr, 18 hr, 24 hr, 3 days, or 1 week.

The eluted nucleic acid can be transferred to a container for storage or further processing, or can be transferred to an assay vessel for characterization.

### Elution of Proteins

Protein can be eluted from a matrix, e.g., a matrix described herein. A sample, e.g., a biological sample, can be stabilized on a stabilization matrix capable of stabilizing a protein, e.g., as described herein, prior to elution. The elution can comprise contacting the stabilization matrix with an elution buffer. The contacting can comprise incubating (e.g., with agitation) the stabilization matrix in the elution buffer to elute the protein from the stabilization matrix. Before elution, the stabilization matrix can be contacted with a binding and or wash buffer.

The elution can be performed on at least a portion of the stabilization matrix comprising a sample, e.g., a dried biological sample. In some cases, a portion of the stabilization matrix can be separated from the rest of the stabilization matrix and used for further processing. In some cases, the portion is more than 5%, 15%, 25%, 35%, 45%, 55%, 65%, 75%, 85%, or 95% of the stabilization matrix. In some cases, the portion is less than 5%, 15%, 25%, 35%, 45%, 55%, 65%, 75%, 85%, or 95% of the stabilization matrix. The portion of a stabilization matrix can be punched out of the stabilization matrix, and proteins in the separated portions can be eluted. The portion separated for further processing can comprise 100%, or about 90%, 80%, 70%, 60%, 50%, or less of a sample that was applied to the matrix. The punches can be about 0.5, 1, 2, 3, 4, 5, 6, 7, 8, 9, 10, 15, 20, 25, 30, 35, 40, 45, 50, 55, or 60 mm in diameter. The punches can be at most 0.5, 1, 2, 3, 4, 5, 6, 7, 8, 9,10, 15, 20, 25, 30, 35, 40, 45, 50, 55, or 60 mm in diameter. The punches can be from about 10 to about 60, from about 10 to about 60, from about 10 to about 60, from about 10 to about 30, from about 10 to about 20, from about 1 to about 10, from about 2 to about 9, from about 3 to about 8, from about 4 to about 7, from about 3 to about 6, from about 4 to about 5, from about 1 to about 4, from about 1 to about 3, from about or from about 1 to about 2 mm in diameter. In some cases, a stabilization matrix comprising protein is not separated into portions before the nucleic acid is eluted from the stabilization matrix.

Protein can be eluted from the stabilization matrix, or portion of the stabilization matrix, by contacting the stabilization matrix, or portion of the stabilization matrix, with an appropriate elution buffer. The elution buffer can comprise one or more buffering agents, one or more surfactants, one or more polyols, one or more salts, one or more blocking agents, one or more reducing agents, one or more organic solvents, one or more chelating agents, one or more salts, e.g., described herein, or any combination thereof.

The one or more buffering agents can be saline, citrate, phosphate, phosphate buffered saline (PBS), acetate, glycine, tris(hydroxymethyl)aminomethane (tris) hydrochloride, tris buffered saline (TBS), 3-[[1,3-dihydroxy-2-(hydroxymethyl)propan-2-yl]amino]propane-1-sulfonic acid (TAPS), bicine, tricine, 3-[[1,3-dihydroxy-2-(hydroxymethyl)propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid) (PIPES), 3-(N-morpholino)propanesulfonic acid (MOPS), 2-(N-morpholino)ethanesulfonic acid (MES), 2-[[1,3-dihydroxy-2-(hydroxymethyl)propan-2-yl]amino]ethanesulfonic acid (TES), cacodylate, glycine, carbonate, or any combination thereof. The buffering agent can be present at a concentration of from about 0.1 mM to about 500, from about 0.1 mM to about 400 mM, from about 0.1 mM to about 300 mM, from about 0.1 mM to about 200 mM, from about 0.1 mM to about 100 mM, from about 0.1 mM to about 50 mM, from about 0.1 mM to about 25 mM, from about 0.1 mM to about 20 mM, from about 0.1 mM to about 15 mM, from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.1 mM to about 0.9 mM, from about 0.1 mM to about 0.8 mM, from about 0.1 mM to about 0.7 mM, from about 0.1 mM to about 0.6 mM, from about 0.1 mM to about 0.5 mM, from about 0.1 mM to about 0.4 mM, from about 0.1 mM to about 0.3 mM, or from about 0.1 mM to about 0.2 mM. The buffering agent can be present at a concentration of less than 500 mM, less than 400 mM, less than 300 mM, less than 200 mM, less than 100 mM, less than 50 mM, less than 25 mM, less than 20 mM, less than 15 mM, less than 10 mM, less than 5 mM, less than 4 mM, less than 3 mM, less than 2 mM, less than 1 mM, less than 0.9 mM, less than 0.8 mM, less than 0.7 mM, less than 0.6 mM, less than 0.5 mM, less than 0.4 mM, less than 0.3 mM, less than 0.2 mM, or less than 0.1 mM. The buffering agent can be present at about 0.1 mM, 1 mM, 10 mM, 25 mM, or 50 mM. The buffering agent can be present at at least 0.1 mM, 1 mM, 10 mM, 25 mM, or 50 mM.

A one or more surfactants can be an anionic, cationic, nonionic or amphoteric type. The one or more surfactants can be polyethoxylated alcohols; polyoxyethylene sorbitan; octoxynol such as Triton X 100™ (polyethylene glycol p-(1,1,3,3-tetramethylbutyl)-phenyl ether); polysorbates such as Tween™ 20 ((e.g., polysorbate 20) or Tween™ 80 (polysorbate 80); sodium dodecyl sulfate; sodium lauryl sulfate; nonylphenol ethoxylate such as Tergitol™; cyclodextrins; or any combination thereof. The surfactant can be present at a concentration of less than 0.001%, less than 0.005%, less than 0.01%, less than 0.015%, less than 0.02%, less than 0.025%, less than 0.03%, less than 0.035%, less than 0.04%, less than 0.045%, less than 0.05%, less than 0.055%, less than 0.06%, less than 0.065%, less than 0.07%, less than 0.075%, less than 0.08%, less than 0.085%, less than 0.09%, less than 0.095%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 0.1%, less than 1%, less than 2%, less than 3% or by volume relative to the total volume of the elution buffer. The one or more surfactants can be present at a concentration of about 0.01%, 0.05%, 0.1%, 0.5%, 1%, 5%, or 10%. The one or more surfactants can be present at a concentration of at least 0.01%, 0.05%, 0.1%, 0.5%, 1%, 5%, or 10%. The one or more surfactants can be present at a concentration of from about 0.01% to 1%, from about 0.05% to 10%, from about 0.10% to 10%, from about 0.15% to 10%, from about 0.2% to 10%, from about 0.25% to 1%, from about 0.3% to 1%, from about 0.35% to 1%, from about 0.4% to 1%, from about 0.45% to 1%, from about 0.5% to 1%, from about 0.55% to 1%, from about 0.6% to 1%, from about 0.65% to 1%, from about 0.7% to 1%, from about 0.75% to 1%, from about 0.8% to 1%, from about 0.85% to 1%, from about 0.9% to 1%, or from about 0.95% to 1%.

The elution buffer can be an aqueous solution having a pH from about 2 to about 10, from about 2 to about 9, from about 2 to about 8, from about 2 to about 7, from about 2 to about 6, from about 2 to about 5, from about 2 to about 4, or from about 2 to about 3. The elution buffer can be an aqueous solution having a pH from about 6 to about 8, from about 6 to about 7.9, from about 6 to about 7.8, from about 6 to about 7.7, from about 6 to about 7.6, from about 6 to about 7.5, from about 6 to about 7.4, from about 6 to about 7.3, from about 6 to about 7.2, from about 6 to about 7.1, from about 6 to about 7, from about 6 to about 6.9, from about 6 to about 6.8, from about 6 to about 6.7, from about 6 to about 6.6, from about 6 to about 6.5, from about 6 to about 6.4, from about 6 to about 6.3, from about 6 to about 6.2, or from about 6 to about 6.1. The elution buffer can be an aqueous solution having a pH of at least about 3, at least about 4, at least about 5, at least about 6, at least about 7, at least about 8, at least about 9, or at least about 10. The pH can be about 6, 6.5, 7, 7.5, 8, or 8.5.

In some instances, the one or more polyols can be a glycol such as ethylene glycol or propylene glycol, or a glycol polymer such as polyethylene glycol (PEG) of various weights such as PEG300, PEG400, PEG600, PEG1000, PEG3000, and PEG6000. In some instances, the one or more polyols can be a sugar. In some cases, the sugar can be sucrose, glucose, fructose, trehalose, maltose, galactose, lactose or any combination thereof. In some instances, the one or more polyols can be a sugar alcohol. In some cases, the sugar alcohol can be glycerol, erythritol, threitol, xylitol, sorbitol and the like.

The one or more salts can be sodium chloride, sodium acetate, sodium bicarbonate, sodium bisulfate, sodium bromide, potassium chloride, potassium acetate, potassium bicarbonate, potassium bisulfate, potassium bromate, potassium bromide, or potassium carbonate. The one or more salts can be at a concentration of about 0.1 mM, 5 mM, 10 mM, 25 mM, 50 mM, 100 mM, 250 mM, 500 mM, or 750 mM. The one or more salts can be at a concentration of less than 0.1 mM, 5 mM, 10 mM, 25 mM, 50 mM, 100 mM, 250 mM, 500 mM, or 750 mM. The one or more salts can be at a concentration of at least 0.1 mM, 5 mM, 10 mM, 25 mM, 50 mM, 100 mM, 250 mM, 500 mM, 750 mM, or 1000 mM.

The one or more blocking agents can be bovine serum albumin, fetal bovine serum, and the like.

The one or more organic solvents can be methanol, ethanol, DMSO, DMF, dioxane, tetrahydrofuran, propanol, isopropanol, butanol, t-butanol, or pentanol, acetone and the like. The elution buffer can comprise less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 99%, or 100% of an organic solvent by volume relative to the total volume of the solution. The concentration of the one or more organic solvents in the elution buffer can be at least 1%, 5%, 10%, 50%, 75%, or 100%. The concentration of the one or more organic solvents in the elution buffer can be about 1%, 5%, 10%, 50%, 75%, or 100%.

The stabilization matrix, or portion of the stabilization matrix, can be contacted with a volume of the elution buffer of about less than 5 μL, less than 10 μL, less than 15 μL, less than 20 μL, less than 25 μL, less than 30 μL, less than 35 μL, less than 40 μL, less than 45 μL, less than 50 μL, less than 55 μL, less than 60 μL, less than 65 μL, less than 70 μL, less than 75 μL, less than 80 μL, less than 85 μL, less than 90 μL, less than 95 μL, less than 100 μL, less than 110 μL, less than 120 μL, less than 130 μL, less than 140 μL, less than 150 μL, less than 160 μL, less than 170 μL, less than 180 μL, less than 190 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1,000 μL less than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, or less than 10 mL. The stabilization matrix, or portion of the stabilization matrix, can be contacted with about 0.1 mL, 0.2 mL, 0.5 mL, 0.7 mL, 1 mL, 2 mL, 5 mL, 7 mL, or 10 mL of elution buffer. The stabilization matrix, or portion of the stabilization matrix, can be contacted with at least 0.1 mL, 0.2 mL, 0.5 mL, 0.7 mL, 1 mL, 2 mL, 5 mL, 7 mL, or 10 mL of elution buffer.

The volume of elution contacted with the stabilization matrix can be dependent on the surface area of the stabilization matrix. The amount of elution buffer can be less than 1 μL/mm², less than 2 μL/mm², less than 3 μL/mm², less than 4 μL/mm², less than 5 μL/mm², less than 6 μL/mm², less than 7 μL/mm², less than 8 μL/mm², less than 9 μL/mm², less than 10 μL/mm², less than 12 μL/mm², less than 14 μL/mm², less than 16 μL/mm², less than 18 μL/mm², less than 20 μL/mm², less than 25 μL/mm², less than 30 μL/mm², less than 35 μL/mm², less than 40 μL/mm², less than 45 μL/mm², less than 50 μL/mm², less than 55 μL/mm², less than 60 μL/mm², less than 65 μL/mm², less than 70 μL/mm², less than 75 μL/mm², less than 80 μL/mm², less than 85 μL/mm², less than 90 μL/mm², less than 95 μL/mm², less than 100 μL/mm², less than 150 μL/mm², less than 200 μL/mm², less than 250 μL/mm², less than 300 μL/mm², less than 350 μL/mm², less than 400 μL/mm², less than 450 μL/mm², less than 500 μL/mm², less than 550 μL/mm², less than 600 μL/mm², less than 650 μL/mm², less than 700 μL/mm², less than 750 μL/mm², less than 800 μL/mm², less than 850 μL/mm², less than 900 μL/mm², less than 950 μL/mm², or less than 1,000 μL/mm². In some cases, the amount of elution buffer can be from about 10 μL/mm² to about 1,000 μL/mm², from about 10 μL/mm² to about 900 μL/mm², from about 10 μL/mm² to about 800 μL/mm², from about 10 μL/mm² to about 700 μL/mm², from about 10 μL/mm² to about 600 μL/mm², from about 10 μL/mm² to about 500 μL/mm², from about 10 μL/mm² to about 400 μL/mm², from about 10 μL/mm² to about 300 μL/mm², from about 10 μL/mm² to about 200 μL/mm², from about 10 μL/mm² to about 100 μL/mm², from about 10 μL/mm² to about 90 μL/mm², from about 10 μL/mm² to about 80 μL/mm², from about 10 μL/mm² to about 70 μL/mm², from about 10 μL/mm² to about 60 μL/mm², from about 10 μL/mm² to about 50 μL/mm², from about 10 μL/mm² to about 40 μL/mm², from about 10 μL/mm² to about 30 μL/mm², or from about 10 μL/mm² to about 20 μL/mm².

The protein can be eluted from the stabilization matrix by incubating the stabilization matrix in the elution buffer in the presence or absence of agitation from an agitation source. An agitation source can be a rocker, vortexer, mixer, shaker and the like. In some cases, an agitation source can be set to a constant speed. The speed can be less than 1 rotations per minute (rpm), less than 5 rpm, less than 10 rpm, less than 15 rpm, less than 20 rpm, less than 25 rpm, less than 30 rpm, less than 35 rpm, less than 40 rpm, less than 45 rpm, less than 50 rpm, less than 55 rpm, less than 60 rpm, less than 65 rpm, less than 70 rpm, less than 75 rpm, less than 80 rpm, less than 85 rpm, less than 90 rpm, less than 95 rpm, less than 100 rpm, less than 150 rpm, less than 200 rpm, less than 250 rpm, less than 300 rpm, less than 350 rpm, less than 400 rpm, less than 450 rpm, less than 500 rpm, less than 550 rpm, less than 600 rpm, less than 650 rpm, less than 700 rpm, less than 750 rpm, less than 800 rpm, less than 850 rpm, less than 900 rpm, less than 950 rpm, less than 1,000 rpm, less than 1,500 rpm, less than 2,000 rpm, less than 2,500 rpm, less than 3,000 rpm, less than 3,500 rpm, less than 4,000 rpm, less than 4,500 rpm, less than 5,000 rpm, less than 5,500 rpm, less than 6,000 rpm, less than 6,500 rpm, less than 7,000 rpm, less than 7,500 rpm, less than 8,000 rpm, less than 8,500 rpm, less than 9,000 rpm, less than 9,500 rpm, or less than 10,000 rpm. The speed can be about 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm. The speed can be at least 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm.

The elution can be performed at a temperature of about 0° C., less than 1° C., less than 2° C., less than 3° C., less than 4° C., less than 5° C., less than 6° C., less than 7° C., less than 8° C., less than 9° C., less than 10° C., less than 11° C., less than 12° C., less than 13° C., less than 14° C., less than 15° C., less than 16° C., less than 17° C., less than 18° C., less than 19° C., less than 20° C., less than 21° C., less than 22° C., less than 23° C., less than 24° C., less than 25° C., less than 26° C., less than 27° C., less than 28° C., less than 29° C., less than 30° C., less than 31° C., less than 32° C., less than 33° C., less than 34° C., less than 35° C., less than 36° C., less than 37° C., less than 38° C., less than 39° C., less than 40° C., less than 45° C., less than 50° C., less than 55° C., less than 60° C., less than 65° C., less than 70° C., less than 75° C., less than 80° C., less than 85° C., less than 90° C., less than 95° C., or about 100° C. The elution can be performed at a temperature of from about 10° C. to about 100° C., from about 10° C. to about 95° C., from about 10° C. to about 90° C., from about 10° C. to about 85° C., from about 10° C. to about 80° C., from about 10° C. to about 75° C., from about 10° C. to about 70° C., from about 10° C. to about 65° C., from about 10° C. to about 60° C., from about 10° C. to about 55° C., from about 10° C. to about 50° C. from about 20° C. to about 50° C., from about 20° C. to about 48° C., from about 20° C. to about 46° C., from about 20° C. to about 44° C., from about 20° C. to about 42° C., from about 20° C. to about 40° C., from about 20° C. to about 38° C., from about 20° C. to about 36° C., from about 20° C. to about 34° C., from about 20° C. to about 32° C., from about 20° C. to about 30° C., from about 20° C. to about 28° C., from about 20° C. to about 26° C., from about 20° C. to about 24° C., or from about 20° C. to about 22° C. The elution be performed at about 10° C., 20° C., 25° C., 30° C., 37° C., 50° C., or 65° C.

The elution (e.g., agitation) can be performed for less than 1, less than 5, less than 10, less than 15, less than 20, less than 25, less than 30, less than 35, less than 40, less than 45, less than 50, less than 55, or less than 60 minutes. The elution can be performed for less than 0.5, less than 1, less than 1.5, less than 2, less than 2.5, less than 3, less than 3.5, less than 4, less than 4.5, less than 5, less than 5.5, less than 6, less than 6.5, less than 7, less than 7.5, less than 8, less than 8.5, less than 9, less than 9.5, less than 10, less than 10.5, less than 11, less than 11.5, or less than 12 hours. The elution can be performed for less than 0.1 days, less than 0.2 days, less than 0.3 days, less than 0.4 days, less than 0.5 days, less than 0.6 days, less than 0.7 days, less than 0.8 days, less than 0.9 days, less than 1 days, less than 1.5 days, less than 2 days, less than 2.5 days, less than 3 days, less than 3.5 days, less than 4 days, less than 4.5 days, less than 5 days, less than 5.5 days, less than 6 days, less than 6.5 days, or less than 7 days. The elution (e.g., agitation) can be performed for about 0.25 hr, 0.5 hr, 1 hr, 2 hr, 5 hr, 10 hr, 12 hr, 18 hr, 24 hr, 3 days, or 1 week. The elution (e.g., agitation) can be performed for at least 0.25 hr, 0.5 hr, 1 hr, 2 hr, 5 hr, 10 hr, 12 hr, 18 hr, 24 hr, 3 days, or 1 week.

The eluted protein can be transferred to a container for storage or further processing, or can be transferred to an assay vessel for characterization.

### Detection

In some embodiments, detection of an analyte from the sample comprises the detection of a tag attached to the analyte. In some embodiments, the analyte is a nucleic acid or a protein. In some embodiments, the nucleic acid or protein has been eluted from the matrix prior to the detection. In some embodiments, the nucleic acid or protein has been concentrated prior to the detection.

In some embodiments, a nucleic acid or a protein is labeled with a tag. In some embodiments, two or more nucleic acids or proteins are labeled with a two or more tags, in order to distinguish each nucleic acid or protein tagged. In some embodiments, a tag is added to the nucleic acid or protein of a sample before the sample contacts the matrix. In some embodiments, a tag is added to the nucleic acid or protein of a sample after the sample contacts the matrix.

In some embodiments, the tag is a dye. Dyes suitable for labeling nucleic acids can include those that are known in the art. The dye can be a fluorescent dye. In some cases, the dye is Cy3 or Cy5. Exemplary labels include, but are not limited to, fluorophores, nanoparticles (e.g., gold nanoparticles), quantum dots, radiolabels, magnetic particles, barcodes (e.g., nucleic acid barcodes), active sites, binding sites, FRET-capable labels, hydrophobic species, hydrophilic species, antibodies, aptamers. In some embodiments, labels are detectable themselves, or allow binding of another detectable species. A nucleic acid can be labeled with nucleic acid barcodes which can subsequently be amplified and detected.

In some embodiments, detection of an analyte in a sample comprises detection of a label attached to the analyte. In some embodiments, the label is detected after the analyte has been eluted from the matrix. Detection of the tag can include, but is not limited to, optical detection (including FRET), electrical detection, magnetic detection, radiolabel detection, sequencing, size detection, surface plasmon resonance (SPR), Raman spectroscopy, and mass spectrometry.

### Uses of Matrices

A sample can be applied to one matrix (e.g., one layer). A sample can be applied to a top matrix, and at least part of the sample can contact a second matrix, e.g., a second matrix below the top matrix. Matrices can be stacked, e.g., with about, or at least 2, 3, 4, 5, 6, 7, 8, 9, 10, 20, 50, or 100 layers. The stack can comprise the same matrix, e.g., each matrix in the stack can have the same composition. The stack can comprise matrices with different compositions, e.g., a matrix configured for stabilizing a nucleic acid can be on top of a matrix configured to stabilize protein, or vice versa. The types of matrices in the stack an alternate; e.g., a matrix configured to stabilize a protein, a matrix configured to stabilize a nucleic acid, followed by a matrix configured to stabilize a protein, etc. The volume applied to a top matrix can pass through at least 2, 3, 4, 5, 6, 7, 8, 9, or 10 layers of matrices. Using multiple matrices can increase yield of recovery of a desired biomolecule, e.g., nucleic acid or polypeptide.

A sample can pass through a plurality of matrices, but the matrices do not contact each other. For example, a sample can be applied to a matrix configured to stabilize a nucleic acid, the nucleic acid can be eluted from the matrix configured to stabilize the nucleic acid and be applied to a matrix configured to stabilize a protein. The matrix configured to stabilize the protein can be used, e.g., to remove contaminates, e.g., protein contaminates from the nucleic acid. A sample can be applied to a matrix configured to stabilize a protein, and the protein can be eluted from the matrix configured to stabilize the protein and be applied to a matrix configured to stabilize a nucleic acid. The matrix configured to stabilize the nucleic acid can be used, e.g., to remove contaminates, e.g., nucleic acid contaminates, from the protein.

### Certain Terminology

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. The term “about” used herein can also mean within a margin of 10% greater than or 10% less than a given value. Where particular values are described in the application and claims, unless otherwise stated the term “about” should be assumed to mean an acceptable error range for the particular value.

The terms “individual,” “patient,” or “subject” can be used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms can refer to human or animal subjects.

The terms “analyte,” “biologically sourced analyte,” or “target component” as used herein, can refer to the one or more biological molecules that can be detected or tested for, in a given diagnostic test. The target component for a particular test can need to be adequately preserved and stabilized for good quality diagnostic results.

The terms “extraction” and “stabilization” used herein, can refer to the stabilization of one or more components of the sample upon contact with the solid stabilization matrix. In some embodiments extraction and stabilization of the sample do not require additional drying steps.

The terms “substrate,” “matrix”, “stabilization matrix,” “solid support matrix,” or “solid matrix” can refer to any solid matrix, or the sample separation component herein described. Substrate can be any solid material including one or more absorbent materials which can absorb a fluidic sample, such as blood.

In a non-limiting example, a sample is a human sample. In another non-limiting example, the samples can include plant or fungal samples. A sample can be blood, plasma, serum, urine, saliva, tissue, hair, skin cells, semen, cerebrospinal fluid, feces, sputum, bone marrow, a suspension of cells, or a suspension of cells and viruses. A sample can be a solution comprising nucleic acids, proteins, or a combination hereof. The nucleic acids can be RNA, DNA, or a combination thereof. These solutions can be utilized in various research, diagnostic, or other analytical applications where it can be desirable to concentrate an analyte of interest, which can be located, or suspected of being located, in the sample.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### EXAMPLES

### Example 1: A Device for Collecting and Storing a Blood Sample

A subject enters a clinic to have her blood drawn. A medical practitioner trained in taking venous blood uses a syringe and standard needles to collect a 5 ml sample from the subject. The sample is drawn from the needle and syringe into a rubber capped vacutainer tube. Within the vacutainer tube is a high surface area per unit volume matrix impregnated with dried reagents, which include: a metal thiocyanate salt, a reducing agent, a buffer, and an RNase inhibitor. The vacutainer contains a pouch comprising dessicant. The liquid blood sample enters the vacutainer and upon exposure to the matrix the dried reagents are rehydrated and drawn into the liquid sample. While this is happening, the sample is also drawn into the matrix. The matrix absorbs the liquid sample creating a driving force across the reagents on the surface, and selectively stabilizing one or more of the sample components as the blood sample is drawn from the patient. After the 5 ml sample is collected from the patient, the vacutainer tube with the sample is stored under ambient conditions. The sample is stored for 3 weeks, and the sample is removed from the tube. RNA from the sample is analyzed and it is determined that the RIN score for the RNA is 9.

### Example 2: Impregnation of a Matrix with Melezitose

Reagents: 31-ETF are from GE Healthcare. Paper substrates are impregnated with melezitose and other reagents by dipping cellulose paper (Whatman 31ETF) in warmed solutions of the appropriate formulations followed by drying the substrate using line oven conveyors. The dried substrates are then sealed in Mylar bags with desiccant until further testing. Four dipping formulations are prepared: (1) a 15% (on a weight-per-volume basis) melezitose solution, (2) a standard FTA solution also containing 15% melezitose. The standard FTA components comprise the following (on a weight-per-volume basis): 0.24% EDTA, 1.63% sodium dodecyl sulfate (SDS), 1.61% Tris buffer salt, and 0.56% Uric acid, (3) a 5% (on a weight-per-volume basis) Ficoll PM400, and (4) a solution containing the following percent sub-components: 6.5% melezitose, 4.2% Ficoll PM 70, and 4.2% Ficoll PM400.

### Example 3: A Blunt-End Repair Reaction Reagents Stabilized on a Matrix

To perform blunt-end repair on fragmented DNA and 5′ end phosphorylation of the blunted fragments, solid support matrices stabilizing sample preparation reagents therein are deployed to a user. In the first solid support matrix configured to selectively stabilize nucleic acids, dNTPs and adenosine triphosphate (ATP) are selectively stabilized therein. In the second solid support matrix configured to selectively stabilize protein, T4 DNA polymerase, Klenow DNA polymerase, and T4 polynucleotide kinase are selectively stabilized therein. Both solid support matrices are then rehydrated with a sample containing DNA. Following rehydration, the rehydrated solid support matrix can be incubated in a thermal cycler for 30 minutes at 20° C. The resulting blunted and 5′ phosphorylated DNA strands are then eluted from the solid support matrix using an elution buffer.

### Example 4: Concentration of Protein from a Blood Sample

A protein in a human blood sample is concentrated in the following example. A solid support matrix is deployed to a user. A sample of blood (0.5 mL) is applied to the solid support matrix. The blood is allowed to dry. The solid support matrix is washed with 2 mL of washing buffer. To isolate a tumour necrosis factor a (TNFa), Anti-TNFa antibody is then applied to the solid support matrix thereby binding TNFa. The solid support matrix is washed with 2 mL of washing buffer. The component and/or affinity molecule are then eluted off of the solid support matrix using 250 μL of elution buffer. The total eluted volume that comprises the TNFa analyte is less than 250 μL.

### Example 5: Methods and Kits for Tagged Sample Collection and Preparation

Sample acquisition and stabilization components are deployed to end users. The sample stabilization components are specifically configured for a designated set of diagnostic tests; in particular they are configured for specific target component. For example, DNA, RNA and protein target components each have different substrate composition and identifying color. Tests configured for an RNA target component have a red stabilization matrix, tests configured for DNA have a green stabilization matrix, and tests configured for a protein target component have a blue stabilization matrix. Furthermore, each deployed system has a unique barcode; this barcode is used to sort and separate the samples by type (RNA/DNA/Protein) for hatching of the samples, and for connecting the sample results with the identifying information corresponding to the donor from which the sample came. The sample stabilization components have multiple barcode labels, one that remains permanently fixed to the sample stabilization component and several removable adhesive labels for easy processing. The labels display the unique barcode associated with the deployed system. After the sample acquisition and stabilization system has been used to acquire the sample, the system is returned to a facility where it is collected. The fixed barcode is used to mechanically separate the received systems by substrate type. For example, all the sample stabilization components with red stabilization matrix are collected together and assembled into batches of 96. Assays are performed based on the sample color tag. A batch of 96 red samples are collected together, two labels from each of the samples are transferred to two sets 96 RNase-free tubes each assembled in order based on the order they were scanned in. Each rack of 96 labeled sample tubes are set-up and organized identically. The sample stabilization components for each of the 96 samples is opened in the order they were scanned in and the red colored substrate is removed and placed, like a filter, onto a rim disposed within the RNase-free sample tubes with the corresponding sample barcode label. This is done until the substrate for each of the 96 samples is in the correctly labeled and corresponding 96 sample tubes. A “red kit” designed for the target components from the red substrate is opened, revealing red-topped containers of RNase-free PCR buffers, reagents and other molecular components, the bottles each have step numbers on them as well, so that they can be easily and quickly used to perform the different treatment steps for the red samples. An aliquot of the “step 1 red reagent” is added to the top of the red colored substrate in each of the 96 labeled tubes, the tubes are closed and the reagents are left for a few minutes to soak into the substrate. After the reagents and buffers have soaked in, the sample is centrifuged-driving the contents of the substrate into the liquid solution that forms at the bottom of the sample tube. Another aliquot of the “step 1 red reagent” is added again to the top of the substrate and the centrifugation is repeated. The tubes are opened and the solid substrate is removed from each sample; then an aliquot of “step 2 red reagent”, comprising a buffered solution containing DNA molecules that are partially double-stranded with a single stranded region that is complementary to target component RNA, is added to the liquid solution. The tubes are closed and placed in a PCR machine at a temperature that encourages Brownian motion without inducing denaturation of the double stranded DNA molecule; at this temperature RNA from the substrate hybridizes with the single stranded region of the DNA molecule. The DNA molecule has a promoter for RNA polymerase within the double stranded region and the 3′ overhang of the single stranded region has a string of thymidine residues. The poly(A) tails of the 3′end of messenger RNA (mRNA) from the red substrate hybridize with the thymine residue overhangs of the DNA molecule. The tubes are then opened and an aliquot of the “step 3 red reagent” comprising ligase enzymes and buffer, is added to the tube. The samples are heated and ligation occurs between the mRNA and the double stranded DNA molecule, forming a double stranded RNA-DNA molecule with the entire mRNA incorporated as one strand of the molecule. The tube is opened and an aliquot of “step 4 red reagent” comprising RNA polymerase is added to each of the 96 tubes, the tubes are closed and 32 PCR cycles of repeated denaturation and annealing are run on the sample to amplify the RNA templates and produce a library of anti-sense cRNA. An aliquot of the amplified cRNA PCR product is transferred to each of the corresponding 96 empty labeled sample tubes, and an aliquot of “step 1 red reagent” is added to the second rack of 96 empty labeled tubes. Steps 2-4 are repeated resulting in ligation and polymerization, this time the resulting in sense strands of the mRNA. These sense strands are then sequenced using standard sequencing protocols, the results are analyzed using standard gene expression profile methods and the barcode number is used as an identifier to determine the donor associated with the given results.

Other kits for performing similar batches of analysis also available; a green kit works for green substrate components which selectively stabilize DNA and blue kits work for blue substrate components which selectively stabilize proteins. Different methods are used to treat samples from each of these different substrates.

### Example 6: Kit with Lancet and Tourniquet with Sample Separation Component

A kit is deployed to an end user or donor. The kit comprises a crystalline-activated pouch for warming hands, a lancet, a tourniquet, alcohol pads, gauze, pressure activated lancet, a self-filling capillary and a sample stabilization component with integrated sample separation unit and sample stabilization matrix. The end user warms donor hands to encourage stimulation of blood flow prior to lancing; this is accomplished by activating a crystalline-activated hand-warmer pouch and holding it between digits of the hand. The donor's hands are relaxed and positioned below the heart and muscles, while the donor sits comfortably in a chair with hand and arms loosely positioned on the arm of the chair. A tourniquet is placed on the donor's non-dominant hand and a site is selected on the donor's middle finger. A rubber band tourniquet is wrapped around the last digit of the finger and then twisted to continue to loop around the finger several times creating a tourniquet. A loop is left available for easy removal. Pressure builds at the fingertip and the fingertip appear slightly red and engorged. Sterilization of the sample site is done; first a side of the fingertip is chosen and then an alcohol pad is swiped past the area before the area is dried with a piece of sterile gauze. The donor holds and pulls on the free loop of the tourniquet during lancing. Lancing process can depend on the type and source of lancet provided. The protective cap of the lancet is removed and the lancet is placed toward the side of the sterilized finger. The lancet is placed to avoid the center of the fingertip, which is calloused and contains a higher density of nerve endings. The lancet is pressed down until the spring in the lancet is engaged and a clicking noise is heard indicating that the skin has been pierced. The first evidence of blood is immediately removed after lancing, and mild but constant pressure is applied to the finger. A self-filling capillary is held horizontal to the incision site and touched against the forming blood droplet using the self-filling capillary (e.g. Microsafe®, Safe-Tee Clinical Products, LLC, Ivyland PA), the capillary self-fill to a black line printed on the plastic shaft and then self-stops. A plastic bulb is present, and it is not depressed during the filling step. When the collected blood reaches the black line and stops filling, pressure is be withdrawn from the fingertip, and the free loop of the rubber band is released to reduce the pressure of the finger tourniquet. Blood is dispensed to the sample stabilization component; the sample stabilization component is placed on a flat surface, and the blood on the outside of the capillary is wiped with clean with sterile gauze. The filled capillary is held upright over the bottom of the sample stabilization component and the collected sample is being dispensed slowly and evenly pressing on a plastic bulb of the filled capillary. The capillary is fixed in place over the bottom of the sample stabilization component while dispensing. The capillary is discarded when all blood is dispensed onto the sample stabilization component. Post-procedure, the blood sampling component is left undisturbed while the finger tourniquet is completely removed and the incision site is cleaned. Pressure is applied to the incision using sterile gauze to stop bleeding and the hand is raised above the heart to assist in clotting. The sample stabilization component is left undisturbed for approximately 5-1 0 minutes post-procedure and observed to determine if the blood drop is still raised on the filter and if filter still appears “wet.” In this case a separation component is used, so the raised “wet” droplet of blood is observed, and then straw-color plasma starts to appear on the top of the sample separation component. The appearance of sample separation is used to indicate that the sample can be placed back into a storage container. The blood sampling component is labeled using a barcode label, and left at room temperature. The storage container is sealed and deposited in the mail.

### Example 7: NIPT and Preeclampsia Tests on Small Volume Sample

A patient greater than 9 weeks pregnant enters a medical clinic for prenatal testing. An untrained medical practitioner offers the patient the option of performing the prenatal test on herself. The patient declines, so the untrained medical practitioner administers the test. The untrained medical practitioner collects a sample consisting of less than 5 mL of blood from the patient. The untrained medical practitioner first uses a lancet to puncture the fingertip of a patient, a droplet of blood accumulates on the finger tip of the patient and then through capillary action the sample is drawn into a sample stabilization unit. The device has a sample separation component that permits separation of sample components followed by transfer of the separated fractions to a solid matrix for storage. A fraction of the sample contains cell-Free DNA (cfDNA), including cell-free fetal DNA (cffDNA), and this fraction is separated from another sample fraction, which containing cells, plasma and protein. The cfDNA and cffDNA are collected on one component of a solid matrix located within the sample stabilization unit, and the cells, proteins and plasma are connected on another separable component of the solid matrix.

The cell-free components including cfDNA and cffDNA are collected on the RSM, a solid matrix where any DNA and RNA components can be selectively stabilized. Upon contact with the solid matrix, RNA and DNA components in the cell-free fraction of the cfDNA and cffDNA are selectively stabilized by the RSM. The cells, protein, and plasma components are collected on PSM—a different solid matrix where the protein components are selectively stabilized. Upon contact with the solid matrix, plasma proteins and any other protein components in the sample are selectively stabilized by the PSM.

The collection, stabilization, storage and transport of the sample occur at ambient temperature. At ambient temperature the cell-free components (e.g. cfDNA and cffDNA) are collected on the RSM and the protein/plasma components are collected on the PSM. The sample is dried, stored, and shipped to a lab for analysis at room temperature. At the laboratory the samples are received at ambient temperature.

The RSM and PSM components are transported together in the sample stabilization unit to a laboratory for testing. Upon arrival at the laboratory, the solid matrix components are removed from the sample stabilization unit. At the laboratory facility the RSM and PSM are separated from the device and from each other. The two components of the sample, the RSM component with cfDNA/cffDNA and the PSM component with plasma/protein, are separated from the device and from each other. The RSM component is used to perform one or more non-invasive prenatal genetic tests, and the PSM component is used to test for pre-eclampsia.

The RSM component with the cfDNA and cell-free fetal DNA are transferred to a tube for non-invasive prenatal genetic tests. Specifically, the RSM is used to determine if the 9 week old fetus carried by the patient has any chromosomal conditions (e.g. Down syndrome). The solid matrix containing the fragments of cfDNA and cffDNA are separated from the solid stabilization matrix. Buffer is added to a tube comprising the solid matrix and the tube is vortexed. The tube is then placed into a PCR machine where it is heated before treated with a single stranded ligase (ssLigase). Treatment with the ssLigase forms single stranded DNA circles. Amplification reagents are added to the single stranded DNA circle products, and rolling circle amplification (RCA) reaction is initiated. The RCA produces large quantities of amplified DNA for sequencing.

In some embodiments a PSM component with the protein and plasma is used to determine if the patient is at risk for pre-eclampsia. The solid matrix containing the plasma and protein fractions are transferred to solution for separation. The protein and plasma components of the sample are separated from the PSM, and re-suspended in a liquid substrate.

One or more protein markers are analyzed. For example, protein markers from the sample are subjected to one or more tests. Under some circumstances the protein markers are subjected to activity assays (e.g. ELISA) to test the activity for one or more protein components. In some circumstances a battery of liver function tests are performed. Liver function tests can include prothrombin time, aPTT, albumin, bilirubin, and other tests including tests for total liver proteins the activity levels for key liver enzymes to detect liver function. In other embodiments the platelet count can be analyzed. The platelet count and liver function tests help determine the likelihood that the patient will develop preeclampsia.

### Example 8: Sequential Testing for Coronary Artery Disease

An older obese male patient with a history of avoiding annual medical checkups is informed by his doctor that he is at risk for developing Coronary Artery Disease (CAD). His doctor acknowledges that the patient is unlikely to follow up with regular annual checkups for monitoring the progression of the disease. The doctor suggests that the patient enroll in an at home testing program that will allow the patient to monitor the progress of his disease from the comfort of his home. The patient signs up for the service, which sends him monthly blood tests kits in the mail. Each test kit contains a tourniquet, a pressure-release lancet, a sample collection unit with a sample stabilization component, and return packaging. The patient uses the monthly test kits to collect, stabilize and send off samples of less than 1 mL to a testing facility. The kits are received at the facility where one or more gene expression tests are performed on the samples to determine the state of his condition. The sample results are monitored over time, allowing the progression of the disease to be sequentially assessed relative to previous samples. The patient can monitor the effect of his lifestyles changes of time, and is informed when he is within a risk threshold enabling him to make an informed decision about on-going medical care, and treatment from a medical professional. The regular testing saves the patient time and money.

### Example 9: Sampling Device Comprising Whole Blood and cfDNA

A patient that is 20 weeks pregnant comes into a clinic to have her blood sampled. A sample of blood with a volume between 1 mL and 5 mL is taken from the patient using a device comprising a sample acquisition component and a sample stabilization component. The device comprises a sample acquisition component, through which the sample is extracted. Once the sample is extracted it moves through the sample stabilization component. The sample stabilization component is arranged such that the sample enters the device, and part of the sample undergoes sample separation through the plasma clip before being stabilized on a sample stabilization matrix and another part of the sample moves through the sample stabilization component and is stabilized on the stabilization matrix as whole blood without separation.

The sample stabilization component is packaged, and stored under ambient conditions and then sent to a CLIA laboratory for analysis. The CLIA laboratory receives the sample, and reconstitutes the dried sample into one or more separate samples. The reconstituted samples are prepared for analysis. The CLIA laboratory runs one or more tests on the sample. In one or more of the tests the maternal genomic DNA from the unfiltered whole blood sample is compared with one or more components from the cell free fraction. In one or more of the tests the cells or other components that were separated from the plasma by the plasma clip are compared with one or more components collected with the plasma. The CLIA laboratory can use comparison between different samples taken on the device e.g. whole blood, filtered with plasma, or filtered from plasma, as controls for the one or more tests conducted on the plasma clip.

### Example 10: Surface Area: Blood Capacity of Absorbent Paper Structures

The surface areas and sample (i.e. blood) capacity of a single strip of paper, a paper shaped into a jelly role, and 4 pieces of paper shaped into an accordion structure and placed in a test tube are provided in Table 1. It is assumed that that: 1) these paper structures are placed into test tube with an inner diameter of 13.3 mm, which accommodates a 12 mm wide structure; 2) the height of the structure is 20 mm; and 3) the filter paper strip absorbs 0.43 μL/mm² of blood.

TABLE 1
Surface area and blood capacity of different absorbant paper structures.

| | | Filter Paper | |
|---|---|---|---|
| Absorbent | | Surface | Blood |
| Structure | | area | Capacity |
| Description | Configuration in Test Tube | (mm²) | (μL) |
| Single strip | 12 mm × 20 mm strip when placed vertically | 240 | 103 |
| Jelly Roll | 12 mm × 20 mm jelly roll (max = cylinder | 2,261 | 972 |
| | vol: 3.14*6*6*20 mm³ when placed vertically | | |
| | without spacing between the spiraled paper) | | |
| 4-fold | 12 mm × 20 mm × 4 | 960 | 413 |
| accordion | | | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the invention and that methods and structures within the scope of these claims and their equivalents be covered thereby.

What is claimed is:

1. A method for collecting blood from a subject, comprising:
a. providing a device comprising (i) a sample acquisition component and (ii) a sample stabilization component fluidically connected to the sample acquisition component, wherein upon actuation of the sample acquisition component, the skin of the subject is punctured, thereby releasing the blood from the subject; and
b. collecting the blood in the sample stabilization component, wherein the sample stabilization component comprises a plurality of substrates, and wherein each substrate of the plurality of substrates absorbs less than 50 μL of the blood.

2. The method of claim 1, wherein the plurality of substrates are removable from the sample stabilization component.

3. The method of claim 1, wherein each substrate of the plurality of substrates is a matrix.

4. The method of claim 3, wherein the matrix is composed of one or more layers of material.

5. The method of claim 3, wherein the matrix comprises a porous material.

6. The method of claim 3, wherein the matrix comprises paper.

7. The method of claim 1, wherein the blood flows from one of the plurality of substrates to the next.

8. The method of claim 1, wherein each substrate of the plurality of substrates absorbs less than 40 μL of the blood.

9. The method of claim 1, wherein each substrate of the plurality of substrates absorbs less than 30 μL of the blood.

10. The method of claim 1, wherein each substrate of the plurality of substrates absorbs less than 20 μL of the blood.

11. The method of claim 1, wherein the device further comprises a collection chamber to collect blood not absorbed by the plurality of substrates.

12. The method of claim 11, wherein the collection chamber comprises an absorptive material.

13. The method of claim 1, wherein the skin of the subject is punctured by one or more piercing elements.

14. The method of claim 13, wherein the one or more piercing elements comprise a lancet.

15. The method of claim 13, wherein a vacuum is created upon withdrawal of the one or more piercing elements from the skin of the subject.

16. The method of claim 1, wherein the actuation comprises pushing a button.

17. The method of claim 1, wherein each substrate of the plurality of substrates has a surface area per unit volume greater than 0.14 mm⁻¹.

18. The method of claim 1, wherein a ratio of a volume of the blood absorbed in each substrate to a surface area of the substrate is at least 0.426 μL/mm².

19. The method of claim 1, wherein subsequent to absorption, the blood in each substrate of the plurality of substrates comprises a dried blood spot.

20. The method of claim 3, wherein the matrix is configured to selectively stabilize protein, nucleic acid, or a combination thereof.
